(19) United States
(12) Patent Application Publication
(10) Pub. No.: US 2023/0279511 A1
(43) Pub. Date: Sep. 7, 2023

(54) METHOD AND SYSTEM FOR SALIVA TESTING FOR VIRUS INCLUDING COVID-19

(71) Applicant: THE ROCKEFELLER UNIVERSITY, New York, NY (US)

(72) Inventor: Robert B. Darnell, New York, NY (US)

(21) Appl. No.: 18/015,452

(22) PCT Filed: Jul. 9, 2021
(86) PCT No.: PCT/US2021/041016

§ 371 Date: Jan. 10, 2023

Related U.S. Application Data
(60) Provisional application No. 63/050,155, filed on Jul. 10, 2020.
(60) Provisional application No. 63/135,159, filed on Jan. 8, 2021.
(60) Provisional application No. 63/135,224, filed on Jan. 8, 2021.
(60) Provisional application No. 63/171,749, filed on Apr. 7, 2021.

(51) Int. Cl. C12Q 1/70; B01L 3/00

(52) U.S. Cl. CPC C12Q 1/701; B01L 3/5082; B01L 2200/185; B01L 2300/021

(57) ABSTRACT

The present invention provides methods for rapid and efficient isolation and characterization of nucleic acid, particularly RNA, from small volume and self-collected samples, particularly saliva samples, to determine the presence or absence of infectious agent RNA, particularly viral RNA, particularly infectious viral RNA, particularly coronavirus. The invention provides methods for isolation and evaluation of infectious agent RNA from saliva samples, particularly viral agents, particularly coronavirus. The invention further provides methods and strategies for pooling samples and RNA to determine the presence or absence of infectious agent RNA, particularly viral RNA, particularly infectious viral RNA, particularly coronavirus, with regard to large numbers of samples at one time and in a single pooled test format.

### FIELD OF THE INVENTION

The present invention relates generally to the rapid and efficient isolation, characterization and analysis of nucleic acid, particularly RNA, from small volume and self-collected samples, particularly saliva samples, to determine the presence or absence of infectious agent RNA, particularly viral RNA, particularly infectious viral RNA, particularly coronavirus. The invention further relates to methods and strategies for pooling samples and RNA to determine the presence or absence of infectious agent RNA, particularly viral RNA, particularly infectious viral RNA, particularly coronavirus, with regard to large numbers of samples at one time and in a single pooled test format.

### BACKGROUND OF THE INVENTION

Coronaviruses are a family of viruses that can cause illnesses such as the common cold, severe acute respiratory syndrome (SARS) and Middle East respiratory syndrome (HERS). In late 2019, a new coronavirus, severe acute respiratory syndrome coronavirus 2 (SARS-CoV-2), was identified as the cause of a COVID-19 disease outbreak that originated in China. In March 2020, the World Health Organization (WHO) declared the COVID-19 outbreak a pandemic. By early 2021, the worldwide number of confirmed COVID-19 cases were over 84 million, with over 20 million in the U.S. Deaths worldwide were over 1,800,000. No accepted or approved treatments or vaccines are available.

Signs and symptoms of COVID-19 appear 2 to 14 days after exposure and can include fever, cough, shortness of breath or difficulty breathing, as well as tiredness, aches, runny nose and sore throat. Some people experience the loss of smell or taste. People who are older or who have existing chronic medical conditions, such as heart disease, lung disease or diabetes, or who have compromised immune systems may be at higher risk of serious illness, similar to what is seen with other respiratory illnesses, such as influenza.

The severity of COVID-19 symptoms can range from very mild to severe and some people may have no symptoms at all. In fact, studies have shown that a significant portion of individuals with coronavirus lack symptoms (“asymptomatic”) and that even those who eventually develop symptoms (“pre-symptomatic”) can transmit the virus to others before showing symptoms (Li R et al Science 10.1126/science.abb3221(2020); Rothe C et al (2020) New Engl J Med 382(10):970-971; Zou L et al (2010) New Engl J Med 382(12)1177-1179; Hou X et al., (2020) Nature 584(7821):420-424). Therefore, the virus can spread between people interacting in close proximity—for example, speaking, coughing, or sneezing—even if those people are not exhibiting symptoms.

In the United States, nearly one-third of COVID-19 disease cases are 6 or older and patients over 65 account for nearly half of hospitalizations and a significant majority of deaths, according to CDC reports. Nonetheless, about 20% of infected people ages 20-44 are hospitalized, demonstrating that this is not just a disease of older adults. The incidence of asymptomatic carriers is largely unknown, as these groups, particularly children, have not been systematically studied. Nonetheless, lateral transmission from adults to children to adults has been well documented (Lopez A S et al., 2020, MMWR Morb Mortal Wkly Rep, 69(37):1319-1323, and children less than age 5 have been reported to carry exceedingly high viral titers relative to those 5-17 or 18 and older (Heald-Sargent T et al., 2020, JAMA Pediatrics, 74(9):902-903).

The recent outbreak of new coronavirus SARS-COV2 and the COVID-19 pandemic presents an imminent need for a system, method and approach for rapid and reliable testing of huge numbers of individuals. At the outset of the appearance of COVID-19, particularly in the U.S., there was a lack of reliable and available tests to assess whether an individual was infected. Although testing has improved, there remains a backlog of patients and individuals needing or wanting testing, even with testing available more widely and testing centers opened in many states and locations. The possibility of testing all or most Americans, which would provide knowledge of the valid number of infected individuals, even those that are asymptomatic or presymptomatic, and reduce ongoing transmission, while a reasonable and scientifically important goal, is not presently feasible. More broad-based testing would permit regular and rigorous screening of individuals and facilitate case detection and isolation and determination of the prevalence and contagiousness of undocumented SARS-COV2 infections. Efforts to reopen or maintain businesses, schools, facilities and events have been hampered by the absence and failure of rapid and effective testing utilizing readily collectable samples. The gold standard used by CDC in years past to culture viruses that cause upper respiratory infections, which involves nasopharyngeal swabs (NPS) transported for subsequent culture in viral transport media (VTM), is particularly slow, inefficient and laborious. RNA testing and PCR-based protocols using NPS samples still have sampling issues. NPS collections are uncomfortable, can trigger sneezing which puts others (particularly health care providers) at increased risk, are inherently risky for contamination (and require extreme handling in shipping and laboratory receipt), and are not well suited for repeat testing. In the absence of broad-based rapid and ready testing, and the general unavailability or unreliability of self-testing or self-collection options, individuals are left to follow stay at home guidelines and businesses, schools, facilities are operating inefficiently and often forced to close with even a small number of coronavirus infections and/or wait until the number of infections starts dropping. In contrast, collection of saliva, particularly small volumes (ranges used in various tests range from 0.3 mls ( 1/15^{th} of a teaspoon) as presented here) to 5 ml (which can be a challenge for individuals to produce), can be applied more generally, with lower risk with collection and can even be implemented to regularly or rapidly screen individuals, employees, attendees, students, staff etc.

Testing saliva, which can be easy to obtain, has been held back in large part because it has been challenging to reliably and technically detect viral RNA from this specimen, in contrast to NPS, the original FDA standard, which are difficult to obtain but, when placed in a viral culture media, have been easy to assay. There is a need for straightforward and dependable systems and methods whereby RNA can be isolated from small volume samples, particularly saliva samples, and tested with rapid dependable results, at low cost, and which are reliable to evaluate individuals in a short time, at low cost and using fewer resources and also that are reliably scalable to evaluate up to hundreds or thousands of individuals in a short time, at low cost and using fewer resources. There is a need for methods, systems and approaches to simultaneously evaluate and assess dozens, hundreds and up to thousands of samples or sample RNAs with sensitivity and specificity such that RNA can be evaluated and particularly the presence or absence of viral RNA can be determined in dozens, hundreds or even thousands of samples dependably, rapidly and at low cost, and even in a single test format or platform.

### SUMMARY OF THE INVENTION

The present invention generally relates to methods and systems for RNA isolation and evaluation of RNA virus infection in small volume samples and self-collected samples, particularly in non-invasively collected samples, particularly in saliva samples, wherein the RNA is of sufficient quality and quantity for rapid, efficient and accurate determination of the presence of viral RNA from one or more virus(es) of concern or interest, and particularly where virus is inactivated at the time of collection for safety purposes. In embodiments of the method, a small volume sample, particularly a saliva sample, may be from a patient or individual having a disease or infection or at risk for or suspected of disease or infection. In embodiments of the method, a small volume sample, particularly a saliva sample, may be from an individual for the purpose of determining whether the individual has a disease or infection, particularly a viral infection, particularly an RNA virus infection. In embodiments of the method, a small volume sample, particularly a saliva sample, may be from an individual, or one or more individual, or more than one individuals, and may be for determining whether said individual or one or more of said one or more individuals, have a virus infection, whether or not they are symptomatic for infection. In embodiments of the method, a small volume of saliva is added to a small volume of lysis or RNA stabilization buffer (lysis/RNA stabilization buffer), which can inactivate infectious virus, particularly infectious respiratory virus, particularly coronavirus.

In embodiments of the method a population of individuals, or one individual, or one or more individuals, may be screened, particularly for one or more virus infection or for the presence of or for positivity of one or more virus RNA, by analysis of RNA isolated in accordance with the present method(s). In embodiments of the method a population of individuals, or dozens of individuals, or many dozens of individuals, or a hundred individuals, or hundreds of individuals, may be screened, particularly for one or more virus infection or for the presence of or for positivity of one or more virus RNA, by analysis of RNA isolated in accordance with the present method(s). In embodiments of the method a population of individuals, or one individual, or one or more individuals, may be screened, particularly for one or more virus infection, particularly an RNA virus infection, or for the presence of or for positivity of one or more virus RNA, by analysis of RNA isolated and analyzed in accordance with the present method(s).

In embodiments of the method, a small volume sample, particularly a saliva sample, may be from an individual, or one or more individual, or more than one individuals, or a population of individuals and may be for determining whether said individual or one or more of said one or more individuals, or one or more individuals in a population of individuals have a coronavirus infection, particularly a SARS-CoV-2 infection or COVID-19, whether or not they are symptomatic for infection. In embodiments of the method a population of individuals, or one individual, or one or more individuals, may be screened, particularly for one or more virus infection or for the presence of or for positivity of one or more virus RNA, particularly coronavirus RNA, particularly SARS-CoV-2 virus RNA, by analysis of RNA isolated in accordance with the present method(s). In embodiments of the method a population of individuals, or dozens of individuals, or up to a hundred individuals, or up to hundreds of individuals, may be screened, particularly for one or more virus infection, particularly for coronavirus infection, particularly for SARS-CoV-2 virus infection, or for the presence of or for positivity of one or more virus RNA, particularly of coronavirus RNA, particularly SARS-CoV-2 virus RNA, by analysis of RNA isolated in accordance with the present method(s). In embodiments of the method a population of individuals, or one individual, or one or more individuals, may be screened, particularly for one or more virus infection, particularly COVID-19 infection, or for the presence of or for positivity of one or more virus RNA, particularly coronavirus RNA, particularly including SARS-CoV-2 virus RNA, by analysis of RNA isolated and analyzed or tested in accordance with the present method(s). In embodiments of the method a population of students and school staff including teachers, or one or more students and school staff, may be screened, particularly for one or more virus infection, particularly COVID-19 infection, or for the presence of or for positivity of one or more virus RNA, particularly coronavirus RNA, particularly including SARS-CoV-2 virus RNA, by analysis of RNA isolated and analyzed or tested in accordance with the present method(s).

In accordance with the method, small volume sample(s), particularly saliva sample(s), is collected and combined with a lysis/RNA stabilization solution or buffer. In some embodiments, the lysis/RNA stabilization solution or buffer acts as a suitable transport medium. In some embodiments, the lysis/RNA stabilization solution or buffer inactivates virus in the sample, particularly in the saliva sample, and also stabilizes RNA therein. In some embodiments, the lysis/RNA stabilization solution or buffer acts as a suitable transport medium, inactivates virus in the sample, particularly in the saliva sample, and also stabilizes RNA therein. In some embodiments, the lysis/RNA stabilization solution is capable of lysing the infectious virus and can also lyse cells in the sample and of stabilizing RNA contained in the virus, cells or cell lysate of the sample. In some embodiments, the lysis/RNA stabilization solution is capable of lysing virus and cells in the sample and of stabilizing RNA contained in the cells, virus or cell lysate of the sample in a single step. In embodiments, the sample and lysis/RNA stabilization solution are mixed, vortexed or shaken when combined.

In some embodiments, the sample may be stored or left at room temperature for up to a few or several hours or days prior to RNA isolation, analysis, refrigeration or freezing. In some embodiments, the sample may be stored or left at room temperature for up to a few or several days prior to refrigeration or RNA analysis. In some embodiments, the sample is then stored at ambient temperature or in refrigerated conditions, such as at about 40° F.; or about 4° C. for a brief time. In some embodiments, the sample is resistent to high temperatures, and may be kept in ambient temperatures up to 150° F.; or about 65° C.; for a brief time, such as 15 minutes to 30 minutes. In some embodiments, the sample is then stored in refrigerated conditions, such as at about 40° F. or about 4° C. for a brief time, up to a day or a few or several days. In some embodiments, the sample is then stored in refrigerated conditions, such as at about 40° F.; or about 4° C. for a brief time, up to a day or a few or several days or a week or up to 7 days, or up to 7-10 days, or at most 7-10 days. In some embodiments, the sample may be stored or left at room temperature for up to a few or several hours, up to 2 hours, up to 3 hours, up to 3 or 4 hours, prior to refrigeration or freezing. In some embodiments, the sample may be stored or left at room temperature for up to one or two days, several days, a few or several days, up to 2 days, up to 3 days, up to 3 or 4 days, for 2-5 days, up to 4-6 days, up to a week, prior to refrigeration. In some embodiments, the sample is then stored in refrigerated conditions, such as at about 40° F.; or about 4° C. for a brief time, up to a day or a few or several days. In some embodiments, the sample is stored in a freezer or in frozen temperature conditions, such as at about 30 or 32° F. or about 0° C., either after collection, after brief (2-4 hour) storage at room temperature, or after brief (1-2 day) refrigerated storage. In some embodiments, the sample is stored in a freezer or in ultra frozen temperature conditions, such as at about −70° C., either after collection, after brief (2-5 day) storage at room temperature, or after brief (2-5 day) refrigerated storage. Thus, in accordance with the invention, once the sample is collected and is placed in lysis/RNA stabilization buffer it is very stable. It is stable at room temperature for a few, several, many days, and also tolerates heat (such as heating for 15 min at 65° C.), and can tolerate freeze/thaw as well, without significant impact on RNA yield/sensitivity.

The invention provides a method for profiling and analysis of infectious agent RNA, particularly viral RNA, of saliva samples from one or more patient or individual comprising:
(a) obtaining one or more small volume saliva sample, wherein the sample is collected in or otherwise combined with a lysis/RNA stabilization buffer comprising a soap such as sarkosyl, and a chaotropic salt, such as guanidinine thiocyanate (GSCN), and a low pH, whereby infectious agent, particularly virus, is inactivated and RNA is stabilized;
(b) isolating RNA;
(c) reverse transcribing the RNA to generate cDNA;
(d) specifically amplifying any infectious agent RNA, particularly viral RNA, using primers and probes specific for the target infectious agent or virus; and
(e) determining the presence and identity of infectious agent RNA, particularly viral RNA, whereby primer amplified nucleic acid for the target infectious agent or virus is identified and characterized.

The invention provides a method for profiling and analysis of infectious agent RNA, particularly viral RNA, of saliva samples from one or more patient or individual comprising:
(a) obtaining one or more small volume saliva sample, wherein the sample is collected in or otherwise combined with a lysis/RNA stabilization buffer comprising a soap such as sarkosyl, and a chaotropic salt, such as guanidinine thiocyanate (GSCN), and a low pH or pH less than 7, whereby infectious agent, particularly virus, is inactivated and RNA is stabilized;
(b) isolating RNA;
(c) reverse transcribing the RNA to generate cDNA;
(d) specifically amplifying any infectious agent RNA, particularly viral RNA, using primers and probes specific for the target infectious agent or virus; and
(e) determining the presence and identity of infectious agent RNA, particularly viral RNA, whereby primer amplified nucleic acid for the target infectious agent or virus is identified and characterized.

In an embodiment low pH is less than 7, less than 6, between 5 and 6 pH, pH about 5.5.

In an embodiment, the chaotropic salt is sodium acetate (NaAc). In an embodiment the sodium acetate is at pH 5.5. In an embodiment, additional sodium acetate is added to the sample prior to isolating the RNA. In an embodiment, about 1/10^{th} the sample volume of 3M sodium acetate, pH about 5.5, is added to the sample prior to RNA extraction or prior to isolating the RNA. In an embodiment, RNA extraction or isolation of RNA is conducted with sodium acetate pH 5.5 at about 300 mM.

The invention provides a method for profiling and analysis of infectious agent RNA, particularly viral RNA, of saliva samples from one or more patient or individual comprising:
(a) obtaining one or more small volume saliva sample, wherein the sample is collected in or otherwise combined with a lysis/RNA stabilization buffer comprising guanidinine thiocyanate (GSCN), mild detergent and organic sodium salt whereby infectious agent, particularly virus, is inactivated and RNA is stabilized;
(b) isolating RNA;
(c) reverse transcribing the RNA to generate cDNA;
(d) specifically amplifying any infectious agent RNA, particularly viral RNA, using primers and probes specific for the target infectious agent or virus; and
(e) determining the presence and identity of infectious agent RNA, particularly viral RNA, whereby primer amplified nucleic acid for the target infectious agent or virus is identified and characterized.

In an embodiment, a method for analysis of coronavirus RNA in saliva samples from one or more patient or individual is provided. In an embodiment, a method for analysis of SARS-CoV-2 virus RNA in saliva samples from one or more patient or individual is provided.

In an embodiment, the organic sodium salt is sodium acetate (NaAc). In an embodiment the sodium acetate is at pH 5.5. In an embodiment, additional sodium acetate is added to the sample prior to isolating the RNA. In an embodiment, about 1/10^{th} the sample volume of 3M sodium acetate, pH about 5.5, is added to the sample prior to RNA extraction or prior to isolating the RNA. In an embodiment, RNA extraction or isolation of RNA is conducted with sodium acetate pH 5.5 at about 300 mM.

The invention provides a method for profiling and analysis of infectious agent RNA, particularly viral RNA, of saliva samples from one or more patient or individual comprising:
(a) obtaining one or more small volume saliva sample, wherein the sample is collected in or otherwise combined with a lysis/RNA stabilization buffer comprising guanidinine thiocyanate (GSCN), mild detergent and organic sodium salt whereby infectious agent, particularly virus, is inactivated and RNA is stabilized;
(b) heating the saliva and lysis/RNA stabilization buffer sample briefly for at about 65° C.;
(c) isolating RNA after brief heating in (b);
(d) reverse transcribing the RNA to generate cDNA;
(e) specifically amplifying any infectious agent RNA, particularly viral RNA, using primers and probes specific for the target infectious agent or virus; and
(f) determining the presence and identity of infectious agent RNA, particularly viral RNA, whereby primer amplified nucleic acid for the target infectious agent or virus is identified and characterized.

The invention provides a method for profiling and analysis of infectious agent RNA, particularly viral RNA, of saliva samples from one or more patient or individual comprising:
(a) obtaining one or more small volume saliva sample, wherein the sample is collected in or otherwise combined with a lysis/RNA stabilization buffer comprising guanidinine thiocyanate (GSCN), mild detergent and organic sodium salt whereby infectious agent, particularly virus, is inactivated and RNA is stabilized;
(b) heating the saliva and lysis/RNA stabilization buffer sample briefly at about 65° C.;
(c) adding a protein denaturant and/or reducing agent to the heated sample;
(d) isolating RNA;
(e) reverse transcribing the RNA to generate cDNA;
(f) specifically amplifying any infectious agent RNA, particularly viral RNA, using primers and probes specific for the target infectious agent or virus; and
(g) determining the presence and identity of infectious agent RNA, particularly viral RNA, whereby primer amplified nucleic acid for the target infectious agent or virus is identified and characterized.

In an embodiment of the method, the saliva and lysis/RNA stabilization buffer sample is heated briefly at about 60-70° C., at about 60-75° C., at about 60-80° C., at about 65-70° C., at about 65° C. In an embodiment of the method, the saliva and lysis/RNA stabilization buffer sample is heated for 10-20 minutes, 10-15 minutes, about 15 minutes, about 15-20 minutes, about 15-18 minutes, about 15-17 minutes, 15 minutes.

In an embodiment, the organic sodium salt is sodium acetate (NaAc). In an embodiment the sodium acetate is at pH 5.5. In an embodiment, additional sodium acetate is added to the sample prior to isolating the RNA. In an embodiment, about 1/10^{th} the sample volume of 3M sodium acetate, pH about 5.5, is added to the sample prior to RNA extraction or prior to isolating the RNA. In an embodiment, RNA extraction or isolation of RNA is conducted with sodium acetate pH 5.5 at about 300 mM.

In an embodiment, a protein denaturant which is a reducing agent is added prior to RNA isolation. In an embodiment a reducing agent is added prior to RNA isolation. In an embodiment, a reducing agent capable of breaking down protein disulfide bonds and stabilizing enzymes and proteins is added. In an embodiment, an agent selected from 2-mercaptoethanol, dithiothreitol (DTT), and TCEP (tris(2-carboxymethyl) is added. The protein denaturant or reducing agent is DTT.

In embodiments, reducing agent is added at about 15-20 μl amount per sample plus buffer volume of 400-600 μl. Reducing agent solution of 2-5M reducing agent, 2-5M DTT, about 3M DTT is added. Final concentration of reducing agent such as DTT is on the order of 50-100 mM, 60-80 mM, about 70-80 mM. In an embodiment of the methods, heating the sample and adding reducing agent/protein denaturant such as DTT serves to better liquefy the sample and thin the sample prior to RNA isolation, liquid handling etc. In an embodiment of the methods, heating the sample and adding reducing agent/protein denaturant such as DTT facilitates the sensitivity of the method and analysis for viral RNA, such that the sample(s) can be handled and dependable and reproducible results can be obtained.

In some embodiments of the method(s), the saliva sample volume is less than 500 μl, about 200-400 μl, about 300 μl, about 200-300 μl, about 250 μl, about 100-300 μl, about 150-300 μl, about 100-250 μl. In an embodiment, the sample volume is about 100-300 μl. In an embodiment, a small volume saliva sample volume is about 100-300 μl. In an embodiment, a small volume saliva sample volume is about 300 μl. In embodiments of the system, particularly wherein the system and RNA isolation is of sufficient quantity and sensitivity to permit detection of target RNA or viral RNA in smaller volumes of saliva, even smaller volumes of saliva may be utilized. For example, in accordance with embodiments of the method of the invention, sensitivity at LOD of 0.5 copies virus/μl of saliva sample is achieved. This could permit detection of virus in substantively less volume that about 200-300 μl saliva samples, such as in 100 μl samples or 50 μl samples. In embodiments, provided suitable sensitivity is achieved, the saliva sample volume may be reduced or adjusted, such as to be less than 1000-300 μl, less than 100 μl, less than 50 μl, less than 20 μl, 200-500 nanoliters, as low as 200 nanoliters.

In some embodiments of the method(s), saliva sample is combined with an approximately equal amount of lysis/RNA stabilization buffer. In an embodiment, the saliva sample is combined with about an equal volume of lysis/RNA stabilization buffer. In an embodiment, the saliva sample is combined with a volume of lysis/RNA stabilization buffer of less than 0.5 ml, of about 300 μl to about 500 μl, of about 250-350 μl, about 200-300 μl, of about 300 μl, of 300 μl. In embodiments, such as noted above, the volume of lysis/RNA stabilization buffer combined with saliva sample is less than 300 μl, less than 200 μl, less than 100 μl, etc.

In some embodiments, the patient or individual obtains or collects the small volume sample, particularly the saliva sample. In some embodiments, the sample is self-collected by the patient or individual or by a non-medical person. The sample may be collected at or near the location of analysis, at the location of school, employment, attendance for analysis there or nearby or to be sent elsewhere for analysis, or may be collected at a distance, such as at home, and delivered, mailed, shipped or otherwise transported to a distinct or distant location for analysis. In some embodiments, the patient or individual is assisted by a non-medical person in collection of the sample. In an embodiment, the sample is collected from a patient or individual by a non-medical person, such as a spouse, parent, friend, guardian, etc that is not medically trained or involved in any medical profession. Critically, the invention describes methods to obtain sufficient quality and quantity of RNA for one or more variety of RNA analyses, in particular for viral RNA analysis.

In accordance with the method, small volume sample(s), particularly saliva, is collected and combined with a lysis/RNA stabilization solution. In some embodiments, the lysis/RNA stabilization solution is capable of lysing the cells in the sample and of stabilizing RNA contained in the cells or cell lysate of the sample. In some embodiments, the lysis/RNA stabilization solution is capable of lysing the cells in the sample and of stabilizing RNA contained in the cells or cell lysate of the sample in a single step. In some embodiments, the lysis/RNA stabilization solution is capable of inactivating virus in the sample and of stabilizing RNA in the sample. In some embodiments, the lysis/RNA stabilization solution is capable of lysing the cells in the sample, of inactivating virus, and of stabilizing RNA contained in the sample and/or in the cells or cell lysate of the sample in a single step. In embodiments, the saliva sample and lysis/RNA stabilization solution are mixed, vortexed or shaken when combined. In some embodiments, the saliva sample in lysis/RNA stabilization solution may be stored or left at room temperature for up to a few or several hours prior to refrigeration. In some embodiments, the sample is then stored in refrigerated conditions, such as at about 40° F.; or about 4° C. for a brief time. In some embodiments, the sample is then stored in refrigerated conditions, such as at about 40° F.; or about 4° C. for a brief time, up to a day or a few or several days. In some embodiments, the sample may be stored or left at room temperature for up to a few or several hours, up to 2 hours, up to 3 hours, up to 3 or 4 hours, prior to refrigeration. In some embodiments, the sample is then stored in refrigerated conditions, such as at about 40° F.; or about 4° C. for a brief time, up to a day or a few or several days. In some embodiments, the sample is stored in a freezer or in frozen temperature conditions, such as at about 30 or 32° F. or about 0° C., either after collection, after brief (2-4 hour) storage at room temperature, or after brief (1-2 day) refrigerated storage.

In some embodiments, the saliva sample is collected into a tube or wherein the tube or receptacle for receiving the small volume sample and containing lysis/RNA stabilization solution has a total volume capacity of 1.5 ml or less, 1.2 ml or less, or 1 ml or less, or less than 1 ml, or less than 750 μl, about 600 μl. In an embodiment, the saliva sample is collected into a tube wherein the tube or receptacle for receiving the small volume sample and containing lysis/RNA stabilization solution has a total volume capacity of 1.5 ml or less, such as a microtainer tube.

In some embodiments, the collection tube has one or more appropriate label(s), such as for designating the name or identity of the patient or individual, date of sample collection and time of sample collection. In embodiments, the label may be a label which is written on by the collector or an individual assisting with collection, it may be a printed label generated by personnel or generated by a printer or machine at the time of collection. In embodiments, the label may be a barcode or may comprise one or more barcodes. The label, such as the barcode or one or more barcodes, may particularly allow matching of the individual to the tube. This matching can be done by a number of means, including written or electronic.

The label, or particularly the one or more barcode, may be present on the side of the tube, suitable for reading by a barcode scanner, or a simple camera such as those present on a mobile device, iPad, laptop, etc. Reading of this barcode can be input by an individual at the time of the sample collection, so that the barcode of any one tube is connected electronically to a secure location (e.g. on a password protected individual web site, previously established by registration between the user and the “reader” (for instance, the laboratory doing the analysis)). In that way, the individual doing the collection can input their name/DOB/other information (e.g. clinical status, contact info, etc, as required for clinical assays, e.g. by NYS DOH), and then at the time of collection connect that information to the barcode on their sample collection tube. Subsequent analysis in the laboratory of the sample, particularly the saliva sample, for the presence of any particular or target nucleic acid or RNA, including standard or control nucleic acid or RNA, with or without detection for virus, can be read out and results delivered to the individual whose saliva was collected.

In some embodiments, the lysis/RNA stabilization solution is a chaotropic salt such as guanidinium thiocyanate based or containing solution. In some embodiments chaotropic salts such as guanidinium thiocyanate based lysis buffers may also contain detergents, which synergize to inactivate adventitious agents, lyse cells. Detergents may include sarkosyl, SDS, or other ionic or non-ionic detergents. Kits/lysis solutions containing chaotropic salts such as guanidinium thiocyanate based lysis buffers with or without detergents, are stable, even up to for years. They can be shipped and used at room temperature. They are less toxic than household bleach, and can be mailed with adherence to suitable or such standards.

In some embodiments, any buffers or solutions are made or generated with RNAse free water or buffers.

In an embodiment, the lysis/RNA stabilization buffer comprises guanidinine thiocyanate (GSCN), mild detergent and organic sodium salt and is prepared in nuclease free water. In an embodiment, the lysis/RNA stabilization buffer comprises guanidinine thiocyanate (GSCN), mild detergent and sodium acetate (NaAc) and is prepared in nuclease free water. In an embodiment, the lysis/RNA stabilization buffer comprises guanidinine thiocyanate (GSCN), sarkosyl and sodium acetate (NaAc) and is prepared in nuclease free water. In an embodiment, the lysis/RNA stabilization buffer comprises ganidinine thiocyanate, sarkosyl and sodium acetate, pH between 5 and 6, and is prepared in nuclease free water. In an embodiment, the lysis/RNA stabilization buffer comprises ganidinine thiocyanate, sarkosyl and sodium acetate, pH about 5.5, and is prepared in nuclease free water. In an embodiment, the pH of between 5 and 6 is utilized for stabilization of RNA, which is stable at that pH, versus DNA which is less stable at low pH, pH below 7. DNA is more stable at higher pH compared with RNA. In an embodiment, the lysis/RNA stabilization buffer has a concentration of components comprising GSCN in the range of 4-6M, particularly 5-6M; sarkosyl in a % range of 0.1%-0.5%, particularly 0.2%-0.6%, particularly 0.2% to 0.5%; sodium acetate (NaAc) at less than 200 mM, particularly 150 mM-200 mM, and at a pH of 5-6, particularly below pH 6, particularly pH 5.5. In an embodiment, the lysis/RNA stabilization buffer has a concentration of components comprising 5M GSCN, 0.5% sarkosyl, 175 mM NaAc, pH 5.5. In an embodiment, the lysis/RNA stabilization buffer has a concentration of components comprising 5M GSCN, 0.2% sarkosyl, 150 mM NaAc, pH 5.5. In an embodiment, the lysis/RNA stabilization buffer has a concentration of components comprising 5M GSCN, 0.2% sarkosyl, 150 mM NaAc, pH 5.5 and further comprises an additional denaturing agent. In an embodiment, the lysis/RNA stabilization buffer has a concentration of components comprising 5M GSCN, 0.2% sarkosyl, 150 mM NaAc, pH 5.5 and further comprises dithiothreitol (DTT). In an embodiment, the lysis/RNA stabilization buffer has a concentration of components comprising 5M GSCN, 0.2% sarkosyl, 150 mM NaAc, pH 5.5 and further comprises 2-mercaptoethanol or beta-mercaptoethanol (BME). In an embodiment, the lysis/RNA stabilization buffer has a concentration of components comprising 5M GSCN, 0.5% sarkosyl, 125 mM NaAc, pH approximately 5.5. In In an embodiment, the lysis/RNA stabilization buffer has a concentration of components comprising 5M GSCN, 0.25% sarkosyl, 200 mM NaAc, pH approximately 5.5. In an embodiment, the lysis/RNA stabilization buffer corresponds to DRUL buffer. In an embodiment, the lysis/RNA stabilization buffer corresponds to DRUL2 buffer. In an embodiment, the lysis/RNA stabilization buffer has a concentration of components comprising 5M GSCN, 0.5% sarkosyl, 175 mM NaAc, pH approximately 5.5. In an embodiment, the lysis/RNA stabilization buffer corresponds to or can be denoted as DRUL2 buffer.

RNA isolation may utilize known and applicable methods for rapid and efficient isolation of RNA. In an embodiment, a rapid and highly scalable isolation method and/or system is utilized so as to facilitate fast and cost effective screening of large numbers of samples in a short period of time and to enable rapid determination of results. Quantitative RNA isolation, wherein the maximum amount of RNA is isolated, may not be required particularly if it is desirable to determine the presence or absence of a certain target RNA but wherein the precise amount or quantitation of the RNA may not be required or necessary. In an embodiment, RNA is isolated using a phenol based system and protocol. In an embodiment, RNA is isolated using one or column or precipitation based system and protocol, such as an alcohol or ethanol based precipitation. In an embodiment, RNA is isolated using one or column based system and protocol. In an embodiment, RNA is isolated using one or magnetic bead system and protocol. Suitable magnetic bead systems, magnetic bead reagents, and protocols are available and known to one skilled in the art and may be utilized. Exemplary magnetic bead systems includeBioMerieux NucliSENS® easyMag extraction system, Quiagen QuiaCube automated extraction system, KingFisher MagMAX or SPRI beads, for example as obtained from Bulldog Biosciences, and KingFisher Flex Magnetic Particle Processor, KingFisher Presto, Perkin Elmer Chemagic 360 system, Agena Biosciences, Cepheid, and other systems.

In an embodiment of the method(s), additional sodium acetate is added to the sample prior to isolating the RNA. In an embodiment, about 1/10^{th} the sample volume of 3M sodium acetate, pH about 5.5, is added to the sample prior to RNA extraction or prior to isolating the RNA. In an embodiment, RNA extraction or isolation of RNA is conducted with sodium acetate pH 5.5 at about 300 mM.

In an embodiment of the method(s), a protease is combined with the sample and buffer prior to or during RNA isolation. In an embodiment of the method(s), a protease is combined with the sample during RNA isolation and prior to adding magnetic bead or magnetic bead solution with a magnetic bead system and protocol. In an embodiment of the method(s), a protease is combined with the sample during RNA isolation and prior to phenol or prior to column-based purification with a phenol extraction or column-based system and protocol for RNA isolation.

In embodiments of the method, any suitable and efficacious protease is utilized. For embodiments using protease, the protease may be proteinase K. Proteinase K has half-maximal activity at room temperature (20° C.), although it can be activated by putting the collected sample in hot tap water (typical tap water is set at a maximum of 120° F., which is about 48° C.; Proteinase K is optimally active at ˜37-55° C.). Suitable proteases are known and available in the art. In some embodiments, the sample is contacted and treated with a protease at a temperature above room temperature. In embodiments, the sample and protease are heated for protease treatment. In embodiments, the protease is deactivated after protease treatment by heating at 95° C. In an embodiment, the sample and protease are kept at room temperature or heated to 50-65° C.; or incubated at a temperature of 50-65° C. In an embodiment, the sample and protease, such as proteinase K (20 mg/ml, diluted in sample to a final concentration of 12.5 ug/ml, or 25.0 ug/ml, 37.5 ug/ml, 50.0 ug/ml, or 100 ug/ml) are kept at room temperature or heated to 65° C., and subsequently the protease is deactivated by serial dilution or heating at 95° C. for 10 minutes.

In a particular and preferred embodiment, RNA is purified using magnetic beads, which in an embodiment permits a more readily scalable purification protocol. The magnetic beads may be carboxylated magnetic beads, cabroxylated magnetic SPRI beads. The magnetic beads may be silica coated magnetic beads, silica coated SPRI beads. For example, different versions of KingFisher MagMAX (SPRI) beads are apparently available, one likely carboxylated, the other silica coated, although proprietary. In an embodiment of the present methods, carboxylated magnetic beads, particularly carboxylated SPRI beads, particularly similar or comparable to carboxylated SPRI beads (Bulldog Bio) are utilized and are combined with use of heated saliva samples (for example heated at 65° C.; for about 15 minutes), treated with DTT or an equivalent denaturant, and samples combined with Binding Buffer (BB), with a salt concentration as provided herein and with PEG 8000, to optimize binding to added carboxylated magnetic beads, followed by Proteinase K treatment (added to the BB) digestion at 65° C., followed by washes/dilutions to inactivate the PK, coincident with purification by washing of RNA-bound magnetic beads in 1 ml and then freshly prepared 75% ethanol, followed by elution in a small volume (25 ul to 50 ul) of RNase free ddH2O.

In accordance with the invention, methods and approaches are provided for pooling RNA samples for testing. In embodiments of these methods, RNA samples from up to 100 or more individuals can be combined or pooled for analysis. Multiplexing in alternative pools can further enable the identification of even a single positive among a hundred or even a thousand samples.

In embodiments herein, up to 96 or about 100 samples are pooled and tested simultaneously. In embodiments herein, RNA isolated from up to 96 or about 100 samples are pooled and tested simultaneously. In embodiments herein, multiple pools of 96 or about 100 individuals are resampled in an additive strategy that gives multiplicative power. In embodiments herein, up to 960 or about 1000 samples are pooled and tested simultaneously. In embodiments herein, RNA isolated from up to 960 or about 1000 samples are pooled and tested simultaneously.

In particular aspects herein, RNA is first isolated from saliva samples and then isolated RNA is pooled for analysis. Aliquots of each RNA sample are combined, added to RNA isolation or binding buffer, combined with magnetic beads for further RNA isolation, and reconcentrated. In embodiments, the pooling method concentrates the RNA and reduces the volume containing RNAs from multiple samples, even up to a hundred or hundreds of samples. Pools of dozens, 12, 24, 48, 96, about 100 isolated RNA samples can be combined and then concentrated in a reduced volume for further analysis of the pooled RNA sample.

In accordance with the present invention and pooling embodiments thereof, after isolation of RNA from samples, particularly saliva sample(s), to generate isolated RNA corresponding to each original sample, aliquots of each RNA are combined, added to RNA binding buffer, combined with magnetic beads for further RNA isolation, and reconcentrated. Pools of dozens, 12, 24, 48, 96, about 100, or even hundreds or thousands of isolated RNA samples can be combined and then concentrated in a reduced volume for further analysis of the pooled RNA sample. In an aspect pools of 10, a dozen, 20, 30, 40, 50, dozens, tens etc RNA samples are combined in each and any well(s) of one or more 96 well plate such that 96 times 10, a dozen, 20, 30, 40, 50, dozens, tens are processed and reconcentrated for analysis in a single 96 well plate. Multiplexing this plate and other plates N-dimensionally then provides 96× the well sample numbers for each 96 well plate. In any such embodiment, multiple multiplexed and N-dimensionally processed plates or any such similar multi sample semi- or fully automated processing permits the evaluation of thousands of samples essentially simultaneously. In an embodiment, multiples of about 96, or about 100, or more with more than 10 per well or other such sample containment receptacle can be processed, such that hundreds, thousands, etc of isolated RNA samples can be combined and then concentrated in a reduced volume for further analysis of the pooled RNA sample and evaluated at one time or with a single process or procedure.

In an embodiment, the invention provides a method and system for pooling and simultaneous analysis of dozens, hundreds, thousands of samples for RNA, particularly viral RNA, such as coronavirus RNA, wherein RNA is isolated from a small volume sample, such as a saliva sample, such as using a saliva RNA isolation protocol or method provided herein. The isolated RNA is then combined and pooled for further RNA isolation, RNA concentration and subsequent analysis. In accordance with the pooling method herein, RNAs are pooled, pooled RNAs are combined with a concentrated binding buffer (2× binding buffer), magnetic beads are added, RNA is isolated, and RNA is resuspended in a smaller than original pooled volume. Thus, in accordance with the pooling methods provided herein, isolated RNA is combined, re-isolated, and reconstituted in a reduced volume thereby concentrating the RNA.

In a particularly preferred embodiment of the invention, magnetic beads are utilized for RNA isolation, or as applicable in the pooling methods, RNA re-isolation or RNA concentration. The invention includes a unique binding buffer and protocol for magnetic bead purification of nucleic acid, particularly and including RNA, particularly for RNA isolation from pooled RNA samples. In this instance the binding buffer, denoted 2× binding buffer, comprises PEG 8000, NaCl, Trisodium citrate, Tween 20, HCl, wherein the amount of salt, particularly the concentration of NaCl and the concentration of trisodium citrate is effectively about twice the concentration as in the binding buffer utilized for RNA isolation, e.g. isolation with magnetic beads, for the original RNA isolation direct on the original sample, such as the original saliva sample RNA isolation. In an embodiment, the binding buffer may additionally and preferably include a protease, particularly proteinase K. In an embodiment, binding buffer includes PEG 8000 20% v/v, 4-6M, 4-5M or 4.5M NaCl, 2 mM Trisodium citrate, Tween 20 at about 0.05% v/v, or less than 0.1% v/v, HCl at 0.5-0.8 mM or about 0.5-0.6 mM or about 0.56 mM. In an embodiment, the binding buffer may additionally include proteinase K at about 100-200 μg/ml or 100-150 μg/ml or about 120 μg/ml, 121 μg/ml.

The invention provides a method for pooling RNA for profiling and analysis of infectious agent RNA, particularly viral RNA, of saliva samples from numerous, dozens or hundreds of patients or individuals comprising:
(a) obtaining one or more small volume saliva sample, wherein the sample is collected in or otherwise combined with a lysis/RNA stabilization buffer comprising guanidinine thiocyanate (GSCN), mild detergent and organic sodium salt whereby infectious agent, particularly virus, is inactivated and RNA is stabilized;
(b) heating the saliva and lysis/RNA stabilization buffer sample briefly at about 65° C.;
(c) adding a protein denaturant and/or reducing agent to the heated sample;
(d) adding a binding buffer suitable to promote binding of RNA nucleic acid to a column or magnetic beads;
(e) isolating RNA to generate numerous individual saliva isolated RNA samples;
(f) combining aliquots of numerous individual saliva isolated RNA samples to generate a pooled RNA sample;
(g) adding a binding buffer having a 2× salt (such as each of NaCl and trisodium citrate) concentration compared to the binding buffer of (d);
(h) isolating RNA using magnetic beads to generate a reconcentrated pooled RNA sample;
(i) reverse transcribing the RNA to generate cDNA;
(j) specifically amplifying any infectious agent RNA, particularly viral RNA, using primers and probes specific for the target infectious agent or virus; and determining the presence and identity of infectious agent RNA, particularly viral RNA, whereby primer amplified nucleic acid for the target infectious agent or virus is identified and characterized.

In an embodiment of the method for pooling RNA, protease, particularly proteinase K is added to the binding buffer having a 2× salt concentration in (g). In an embodiment, added proteinase K to the binding buffer facilitates the assay sensitivity and specificity and improves the pooling method.

In an embodiment of the method for pooling RNA, a single positive sample can be detected in a pooled RNA sample in a single test comprising 96 or 100 individual pooled RNA samples, particularly comprising testing of a single multiwell, such as a 96 well plate. In an embodiment of the method for pooling RNA, a single positive sample can be detected and uniquely and specifically identified in a pooled RNA sample comprising 96 or 100 individual pooled RNA samples. In an embodiment of the method for pooling RNA, a single positive sample can be detected and uniquely and specifically identified in a pooled RNA sample comprising 96 or 100 individual pooled RNA samples, wherein identification is provided by plate, row, column in an N dimensional plating system for pooling analysis. In an embodiment of the method, more than one positive sample can be detected in a pooled RNA sample comprising 96 or 100 individual isolated RNA samples. In an embodiment of the method, more than five positive samples can be detected in a pooled RNA sample comprising 96 or 100 individual isolated RNA samples. In an embodiment of the method, ten positive samples can be detected in a pooled RNA sample comprising 96 or 100 individual isolated RNA samples. In an embodiment of the method, numerous positive samples can be detected and uniquely and specifically identified in a pooled RNA sample comprising 96 or 100 individual isolated RNA samples. This detection is multiplexed with multiple plate analyses. Thus if 10 plates are analyzed, detection is determined from among the 10 fold sampling number.

Amplification may utilize known and applicable methods for amplification such as polymerase chain reaction (PCR).

Primers may be directed to one or more specific infectious agent target sequence, particularly viral target sequence. One or more primers directed to one or more targets of infectious agent or viral RNA may be utilized. Forward and reverse primers are typically utilized to specifically amplify the sequence in the region between the primers.

In an embodiment, primers directed to coronavirus are utilized. In an embodiment, primers directed to SARS-CoV-2 virus are utilized. In an embodiment, primers directed to one or more conserved SARS-CoV-2 virus RNA sequence are utilized as well as one or more primer to a general sequence present in any sample RNA are utilized, such as a ribonuclease or housekeeping RNA sequence.

Amplification may utilize a labeled probe, in an embodiment wherein the probe anneals to a specific probe target sequence located between the forward and reverse primers. In a particular embodiment, the probe has or incorporates a reporter or dye. During the extension phase of the amplification process, the 5′ nuclease activity of the DNA polymerase degrades the bound probe. This serves to release the reporter dye from a quencher dye and results in a signal, such as a fluorescent signal. The fluorescence intensity can be monitored and evaluated to determine the presence and/or amount of target sequence, which reflects the amount of amplified sequence and the presence and/or amount of infectious agent target sequence, particularly viral target sequence.

Suitable reporter dye(s) and quenchers or quencher dye(s) are known and available in the art. Examples include reporter dye FAM, Quasar 670HEX, LC610. Examples include quencher or quencher dye BHQ1, IABkFQ, BBQ.

In some embodiments, primers directed to multiple viral RNA target sequences are utilized. The target sequences may be directed to one or more, two or more, two, multiple specific viral RNA target sequences. Primers to a generic or housekeeping sequence present in any infectious agent RNA may be utilized. Suitable and applicable generic or housekeeping sequences present in any infectious agent RNA or viral RNA will be known and available to one skilled in the art. In one such embodiment, primers to RNAseP are utilized as a positive control for example for the presence of viral RNA. In an embodiment, primers are directed to viral RNA N protein, particularly N1 and N2 protein are utilized. In an embodiment, primers directed to viral RNA S protein sequence are utilized and may be utilized additionally or alternatively. In an embodiment, primers directed to coronavirus-specific N1, and/or N2, and/or S protein are utilized. In an embodiment, primers directed to coronavirus-specific N1, and N2 protein are utilized. In an embodiment, primers directed to coronavirus COVID-19 or SARS-CoV-2 virus-specific N1 and N2. In an embodiment a generic viral RNA sequence is used as a target, for example a sequence characteristic or all of a family of viruses, such as to all coronaviruses, or all influenza viruses or all severe acute respiratory syndrome (SARS)-related coronaviruses. In an embodiment, primer sequence directed to the RNA-dependent RNA polymerase (RdRp) gene of all severe acute respiratory syndrome (SARS)-related coronaviruses is utilized. This could be of value since the RdRp gene of SARS-CoV-2 is relatively invariant and thereby resistant to mutation, vs other regions (particularly those encoding the spike protein, where there is evolutionary pressure to mutate and avoid antibody detection). Note this gene is independent of use of a control primer, human RNase P (RP), present as a positive sample control. Exemplary and accepted assy primers include those provided herein, without limitation, including in Tables 1 and 28. Alternative primers are also indicated herein and/or can be constructed by one skilled in the art based on available or known virus sequence, coronavirus sequence, Sars-CoV-2 sequence. For example, a reference SARS-CoV-2 virus genome sequence is provided in NC_045512.

In an embodiment, the virus-specific primers, such as the N1 and/or N2 primers, may or can be adjusted or modified to ensure primer binding and recognition of virus mutants or variants. In an embodiment, the virus-specific primers, such as the N1 and/or N2 primers and/or S primers, may or can be adjusted or modified to ensure primer binding and recognition of virus mutants or variants. In an embodiment, virus-specific primers, such as S primers, may or can be added and incorporated in addition to N1 and N2 primers, and/or any such S primers may or can be adjusted or modified to ensure primer binding and recognition of virus mutants or variants. In any such instance wherein the virus, particularly coronavirus might or does mutate, such that one or more coronavirus variant may need to be screened for and identified, the primers may be adjusted or additional primers may be added so as to ensure that the virus and any and all variants are identified and amplified and can be screened. In embodiments, multiple virus primers, for example multiple N1 or N2 primers may be utilized, as in a multiplex amplification scheme or protocol.

In some embodiments, the isolated RNA is converted to cDNA and may be cloned or a library prepared therefrom or containing or based on the cDNA(s). In some embodiments, the primers can be modified to allow cDNA to be directly sequenced in a high throughput sequencer commensurate with or following amplification. In some embodiments, additional primers may be utilized post assay to amplify certain virus genome or encoding regions, such as to identify or sequence virus variants. These can be direct sequenced using any methods or systems known and available in the art. For example, direct sequencing using Illumna sequencing machines, and appropriate primers may be employed, using systems and protocols described and known by or otherwise available to one skilled in the art.

The invention further provides systems and kits for isolating and evaluating RNA in saliva samples, particularly in self-collected saliva samples, and for use and application of the methods herein. In an embodiment, the systems and kits are for use and application in serial monitoring of a population or of individuals for determining the presence of a virus and/or for detection of virus infection, even in pre-symptomatic and/or asymptomatic individuals. In an embodiment, the the systems and kits are for use and application for determining the presence of a coronavirus. In an embodiment, the the systems and kits are for use and application for determining the presence of COVID-19 virus SARS-CoV-2. In an embodiment, the the systems and kits are for use and application for determining the presence of COVID-19 virus SARS-CoV-2, including identification of variants thereof.

In embodiments, a system or kit is provided for RNA isolation and analysis of small volume saliva samples from a patient or individual comprising:
(a) a means for self-collection of a saliva sample by the patient or individual or by a non medical person, comprising a receptable for a spit or aspirate;
(b) a tube or receptacle for receiving the small volume sample on collection and containing a volume of lysis/RNA stabilization solution whereby virus and/or cells in the sample are lysed and RNA is stabilized;
(c) a bulb or similar transfer device for transferring the saliva sample collected in (a) into the tube containing lysis/RNA stabilization solution in (b), and
(d) one or more appropriate label(s) for designating the name or identity of the patient or individual, date of sample collection and/or time of sample collection.

In embodiments, the label may be a label which is written on by the collector or an individual assisting with collection. In embodiments, the label may be a printed label generated by personnel or generated by a printer or machine at the time of collection. In embodiments, the label may be a barcode or may comprise one or more barcodes. The label, such as the barcode or one or more barcodes, may particularly allow matching of the individual to the tube. This matching can be done by a number of means, including written or electronic.

In an embodiment, the system or kit is provided for RNA isolation and analysis of small volume saliva samples and determination of the presence of viral RNA in a patient or individual. In an embodiment, the system or kit is provided for RNA isolation and analysis of small volume saliva samples and determination of the presence of virus infection in a patient or individual. In an embodiment, the system or kit is provided for RNA isolation and analysis of small volume saliva samples and determination of the presence of coronavirus infection in a patient or individual. In an embodiment, the system or kit is provided for RNA isolation and analysis of small volume saliva samples and determination of the presence of COVID-19 infection or SARS-CoV-2 virus infection in a patient or individual. In an embodiment, the system or kit is provided for RNA isolation and analysis of small volume saliva samples and determination of the presence of COVID-19 infection or SARS-CoV-2 virus infection and/or another virus infection in a patient or individual. In an embodiment, the system or kit is provided for RNA isolation and analysis of small volume saliva samples and determination of the presence of COVID-19 infection or SARS-CoV-2 virus infection and/or influenza virus infection in a patient or individual.

In embodiments, a system or kit is provided for analysis of infectious agent RNA in saliva samples from a patient or individual comprising:
(a) a means for self-collection of a saliva sample by the patient or individual or by a non medical person, comprising a receptable for a wash, spit or aspirate;
(b) a tube or receptacle for receiving the saliva sample on collection and containing a volume of lysis/RNA stabilization solution whereby virus in the sample is inactivated and RNA is stabilized; and
(c) one or more appropriate label(s) for designating the name or identity of the patient or individual, date of sample collection and/or time of sample collection.

In embodiments, the system or kit may further comprise an envelope or mailing container for shipment of the sample to a laboratory or facility for RNA isolation and analysis.

In an embodiment, the tube or receptacle for receiving the small volume saliva sample on collection contains a volume of lysis/RNA stabilization solution whereby cells in the sample are lysed, RNA is stabilized and virus is inactivated.

In embodiments, a system or kit is provided for RNA isolation and analysis of small volume saliva samples from a patient or individual comprising:
(a) a means for self-collection of a saliva sample by the patient or individual or by a non medical person, comprising a receptable for a spit or aspirate;
(b) a tube or receptacle for receiving the small volume sample on collection and containing a volume of lysis/RNA stabilization solution whereby RNA is stabilized and virus is inactivated; and
(c) one or more appropriate label(s) for designating the name or identity of the patient or individual, date of sample collection and time of sample collection.

In embodiments, the system or kit may further comprise an envelope or mailing container for shipment of the sample to a laboratory or facility for RNA isolation and analysis.

In some embodiments, the system or kit may be for RNA analysis and virus infection determination of multiple small volume saliva samples collected in series from a patient or individual over days, weeks or months or from numerous patients or individuals in a single day or over several days or over a week comprising:
(a) a set of numerous means for self-collection of individual small volume saliva samples by the patient or individual or by a non-medical person, each comprising a receptable for a spit or aspirate;
(b) a set of numerous tubes or receptacles each individually for receiving a small volume saliva sample on collection and containing a volume of lysis/RNA stabilization solution whereby RNA is stabilized and virus is inactivated;
(c) numerous appropriate label(s) for designating the name or identity of the patient or individual, date of sample collection and time of sample collection; and
(d) numerous envelopes or mailing containers for shipment of each sample or several samples to a laboratory or facility for RNA isolation and analysis.

In some embodiments of the system or kit, the saliva sample volume is less than 500 μl, about 200-400 μl, about 300 μl, about 200-300 μl, about 250 μl, about 100-300 μl, about 150-300 μl, about 100-250 μl. In an embodiment, the sample volume is about 100-300 μl. In an embodiment, a small volume saliva sample volume is about 100-300 μl. In an embodiment, a small volume saliva sample volume is about 300 μl.

In embodiments, provided suitable sensitivity is achieved, the saliva sample volume may be reduced or adjusted, such as to be less than 1000-300 μl, less than 100 μl, less than 50 μl, less than 20 μl, 200-500 nanoliters, as low as 200 nanoliters.

In some embodiments of the system or kit, the volume of lysis/RNA stabilization solution is less than 1 ml, about 500 μl or less, about 300 μl, about 300 μl or less, about 200-300 μl, or about 250 μl. In an embodiment, the volume of lysis/RNA stabilization solution is about 300 μl, about 300 μl or less, about 200-300 μl, or about 250 μl. In some embodiments of the method(s), saliva sample is combined with an approximately equal amount of lysis/RNA stabilization buffer. In an embodiment, the saliva sample is combined with about an equal volume of lysis/RNA stabilization buffer. Thus, the lysis/RNA stabilization buffer volume may be adjusted to be commensurate or suitable with the volume of saliva collected or required for adequate and sensitive and suitable testing. In embodiments, such as noted above, the volume of lysis/RNA stabilization buffer combined with saliva sample is less than 300 μl, less than 200 μl, less than 100 μl, etc.

In some embodiments of the system or kit, the tube or receptacle for receiving the small volume sample and containing lysis/RNA stabilization solution has a total volume capacity of 1.5 ml or less, 1.2 ml or less, or 1 ml or less.

Other objects and advantages will become apparent to those skilled in the art from a review of the ensuing detailed description, which proceeds with reference to the following illustrative drawings, and the attendant claims.

### BRIEF DESCRIPTION OF THE DRAWINGS

The patent or patent application contains at least one drawing executed in color. Copies of this patent or patent application publication with color drawing(s) will be provided by the U.S. Patent and Trademark Office upon request and payment of the necessary fee.

FIG. 1 depicts sample instructions for at home saliva collection.

FIG. 2 depicts an exemplary grid for reagent set up.

FIG. 3 depicts an exemplary grid for sample set up. NTC indicates no template control. EC indicates exr=traction control. PC indicates positive control.

FIG. 4 depicts clinical evaluation of Primer N1 on nasopharyngeal swab samples proceesed in DRUL buffer and extracted using the phenol method, assayed using the BioRad CFX96 compared with ABI 7500fast platform.

FIG. 5 depicts the correlation of the RU assay with Cephied Xpert Xpress SARS-CoV-2 assay. Y axis shows results (in Ct values) obtained from 30 different positive individuals using DRUL buffer to extract positive samples also assayed (X axis) by NYSDOH using FDA approved Cephied Xpert Xpress SARS-CoV-2 assay. The high R² value indicates extremely robust correlation of sensitivity of sample extraction and downstream RNA analysis with DRUL and the FDA assay across a wide range (4.2 million-fold range) of viral loads in actual clinical samples.

FIG. 6 depicts the DRUL lysis and standard RNA purification assay limitation of detection of SARS-CoV-2 virus at 1 copy per μl.

FIG. 7 depicts a sample instruction sheet for saliva collection for virus testing particularly SARS-COV-2 Testing.

FIG. 8 depicts an exemplary requisition form for virus testing particularly for SARS-Cov-2 Assay 2 testing.

FIG. 9 depicts the DRUL2 assay (version 2) using RNA purification with magnetic beads limitation of detection of SARS-CoV-2 virus at 0.5 copies virus per μl.

FIG. 10 depicts the stability of RNA in DRUL buffer. A evaluation with 229E Primer 1 after overnight storage and 7 days storage at room temperature. B evaluation with 229E Primer 2 after overnight storage and 7 days storage at room temperature. C evaluation with 229 Primer 1 and 229E Primer 2 after storage in saliva alone or saliva plus DRUL buffer at 0° C., 25° C.; and 38° C.

FIG. 11 depicts inactivation of virus in DRUL buffer. A provides Huh7.5 cell viability without virus and with serial dilution of Buffer:229E. B depicts Huh7.5 cell viability over time in DRUL buffer and with and without buffer.

FIG. 12 depicts sensitivity of DRUL buffer compared to commercial NPS swab tests Quest/OP swab and Bioreference/Nasal swab. The DRUL buffer saliva test provided herein detects missed positives in comparison to each of the Quest and Bioreference tests.

FIG. 13 depicts results of the SARS-CoV-2 DRUL2 buffer test using magnetic bead RNA purification. Highly reproducible results have been provided with saliva RP results across 1000s of samples with median Ct ˜25, allows confidence in any single sample, QC (samples with Ct above 2 Std Dev rejected). Sensitivity of the version 2 using magnetic bead purification of RNA from saliva is improved over version 1 using ph/chl or column purification to 0.5 (true) viral copies/ul. The saliva assay found 3 false negatives in commercial NPS tests (BioReliance, Quest)(as shown in FIG. 12) (saliva positive in RU tests). Turnaround Time: RU test TAT now ˜3.5 hrs. Quest:TAT ˜2-4 days. Cost: RU reagents $4/test (Head Count 3) Quest charge/test: $118.

FIG. 14 depicts saliva sample amounts and dropper volume collection and also barcoding on tube for sample collection. One dropper of saliva is added to a tube with barcoding. After addition of the dropper saliva sample the tube should be approximately half full. The barcode is entered on the sample collection form to properly identify and correlate the sample and the individual.

FIG. 15 depicts labeling samples using a bar code label and system. A depicts a scan of the barcode label on a sample as depicted on a cell phone device. B provides a health information survey depiction showing that a barcoded redcap tube with saliva sample has been scanned and entered in a sample survey system. C depicts an example sample collection tube with lysis/RNA stabilization buffer and having a barcode label identifier. D shows a collection or group of 96 sample collection tubes with barcodes indicated as well as a side detail.

FIG. 16 provides an exemplary health information survey for a sample study and evaluation.

FIG. 17 provides an exemplary report of SARS-CoV-2 RNA testing results from self-collected saliva sample wherein the result is negative.

FIG. 18 provides an exemplary report of SARS-CoV-2 RNA testing results from self-collected saliva sample wherein the result is positive.

FIG. 19 provides an exemplary report of SARS-CoV-2 RNA testing results from self-collected saliva sample wherein the result is indeterminate.

FIG. 20 provides an exemplary report of SARS-CoV-2 RNA testing results from self-collected saliva sample wherein the result is invalid.

FIG. 21 depicts Limit of detection (LOD). Determination of LOD with (A) phenol and (B) column-based extraction methods and confirmation of LOD using (C) phenol and (D) column-based extraction methods in 20 replicates and (E) column-based extraction method in 20 separate extractions from unique saliva samples. Black bars=N1 primer, open bars=N2 primer, gray bars=RNase P primer. Error bars=1 standard deviation.

FIG. 22 depicts assay performance with two extraction methods. Specimen matrix spiked with specified concentration of synthetic RNA and extracted using A. phenol and B. column-based methods. Black bars=N1 primer, open bars=N2 primer, gray bars=RNase P primer. Error bars=1 standard deviation.

FIG. 23. Standard curve for the chemically inactivated SARS-CoV-2. Standard curve calculated using the chemically inactivated SARS-CoV-2 in DRUL buffer and extracted with SPRI magnetic beads. Patient 1's viral load at diagnosis corresponds to 134,000 and 255,000 copies per ml for N1 and N2, respectively. Patient 2's viral load at diagnosis corresponds to 400 and 450 copies per ml for N1 and N2, respectively.

FIG. 24. Evidence for vaccine efficacy in Patient 1. A. SARS-CoV-2 pseudovirus (wt) neutralization assay [13]. Relative luminescence units (RLU) normalized to untreated shown for Patient 1. Average serum neutralizing antibody titers (NT50) are from 3 independent experiments, each performed in duplicate. NT50=3209+/−760. B. NT50 values for plasma obtained from recovered patients, 1.3 months [14] and 6.2 months [15] post infection, vaccinated individuals (Moderna or Pfizer-BioNTech) [4]. and Patient 1 (post vaccine and infected) are plotted, with geometric mean in red (bars and numbers), showing evidence for the high levels of neutralizing activity (NT50=3209) from Patient 1.

FIG. 25. Neutralization assay for the Wuhan-1, E484K, and NYC variant. Neutralization assay performed with serum from Patient 1 against pseudoviruses carrying the indicated spikes: wild type (Wuhan-1), E484K mutant, or NYC variant (L5F/T95I/D253G/E484K/D614G/A701V). Shown are averages and standard deviations of three independent experiments, indicating that the E484K substitution alone or together with other substitutions in the NYC variant are not significantly more resistant to neutralization by the serum from patient 1 than the wild type virus.

FIG. 26 depicts results of Saliva SARS-CoV-2 testing using DRUL Pooling. Saliva samples were spiked with inactivated SARS-CoV-2 virus and assayed over a range of dilutions with extracted RNA from clinical saliva samples. Dilutions ranged from 1× (undiluted) to 96× pooled saliva sample RNAs. It is evident that RP values decrease with pooling, likely as a function of successful reconcentration of RP RNA from increasing numbers of pooled samples. SARS-CoV-2 RNA N1 and N2 remained nearly constant over a range of dilutions, with a change in Ct between 1× and 96× of 1.04 Ct increase. This corresponds to a loss of sensitivity of 3% after 96× dilution of SARS-CoV-2 virus.

FIG. 27 A-F depicts and provides illustration of N-dimensional pooling strategy for SARS-CoV-2 testing (Dimension 1, 2 and 3 are shown). In pooling dimension 1 (A and B), about 100 pooled samples are combined in a PCR reaction. In pooling rows from 10 plates, with 96 individuals per plate a total of 960 individual tests can be conducted, for about 1000 pooled samples in 10 PCR reactions. (C) Pooling columns is demonstrated in Dimension 2. (D) Pooling rows is demonstrated in Dimension 3. Pooling results for a single positive precisely detected in triplicate among 1000 pooled samples is depicted in E. Pooling results from up to 2880 individuals to detect a single positive precisely (1 PCR run detecting 1 positive precisely in triplicate) is depicted in F.

FIG. 28 depicts effects of Proteinase K and heat inactivation on Ct. A shows magnetic bead fines (dark brown sediment type material) in wells without proteinase K. Fines are eliminated with proteinase K (PK) at different concentrations. Proteinase K at low concentrations (1.0=55 ug/ml PK). Heat inactivation of PK is not required and may worsen sensitivity. B shows SARS-CoV-2 N1, N2 detection is stable with 96 pooled samples: ˜97% RNA recovery. RP (saliva control) improves with pooling (lower Ct), indicating concentration of saliva RNA is working.

FIG. 29 provides pooling results from 2,880 individuals. SARS-CoV-2 N1 (blue), N2 (orange) detection stable with 96 pooled samples: ˜97% RNA recovery. RP (saliva control) (grey) improves with pooling (lower Ct), indicating concentration of saliva RNA is working.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

As used herein, “RNA” is defined as at least two ribonucleotides covalently linked together. The RNA may be any type of RNA. Examples include mRNA, tRNA, rRNA, shRNA, circRNA, scaRNA, scRNA, snRNA, siRNA or Piwi-interacting RNA, or a pri-miRNA, pre-miRNA, miRNA, snoRNA, long ncRNAs, anti-miRNA, precursors and any variants thereof. Further examples of RNA include RNA of a virus, or RNA sequences derived from a virus genome. Even further examples include RNA of a bacteria. RNA may be single stranded or double stranded, or may contain portions of both double stranded and single stranded sequence. RNA may be synthesized as a single stranded molecule or expressed in a cell (in vitro or in vivo) using a synthetic gene. RNA may be obtained by chemical synthesis methods or by recombinant methods.

RNA may also encompass the complementary strand of a depicted single strand. Many variants of RNA may be used for the same purpose as a given RNA. Thus, RNA also encompasses substantially identical RNA and complements thereof. A single strand provides a probe that may hybridize to a target sequence under stringent hybridization conditions. Thus, RNA also encompasses a probe that hybridizes under stringent hybridization conditions.

As used herein, “pg” means picogram, “ng” means nanogram, “ug” or “μg” mean microgram, “mg” means milligram, “μl” or “μl” mean microliter, “ml” means milliliter, “1” means liter.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be single-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

A “protease” as defined herein is an enzyme that hydrolyses peptide bonds. Conventional proteases may be used. Proteinase K is an example. It is preferred that the specific activity of the protease be high to degrade proteins in what can be a protein-rich sample and thereby protect the RNA from ribonucleases. The specific activity as determined by the Chromozym assay of the protease in the mixture of biological sample and denaturing solution is for example at least about 0.1 U/ml, at least about 1 U/ml, at least about 2.5 U/ml, at least about 5 U/ml, or at least about 10 U/ml. In another embodiment, the specific activity of the protease in the mixture is between 0.1 and 1000 U/ml.

Reference throughout this specification to “one embodiment,” “an embodiment,” “one example,” or “an example” means that a particular feature, structure or characteristic described in connection with the embodiment or example is included in at least one embodiment of the present embodiments. Thus, appearances of the phrases “in one embodiment,” “in an embodiment,” “one example,” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

As used herein, the terms “comprises,” “comprising,” “includes,” “including,” “has,” “having,” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus.

Further, unless expressly stated to the contrary, “or” refers to an inclusive “or” and not to an exclusive “or”. For example, a condition A or B is satisfied by any one of the following: A is true (or present) and B is false (or not present), A is false (or not present) and B is true (or present), and both A and B are true (or present).

In accordance with the invention, methods and approaches are provided herein for isolation and analysis of RNA, particularly of RNA of an infectious agent, particularly of viral RNA, from saliva samples. In accordance with the methods, saliva samples are utilized for rapid and effective determination of viral RNA. The methods are applicable to virus detection in the instance of a sudden outbreak and in the case of an epidemic or pandemic. The methods are applicable for evaluation of coronavirus. Detailed and specific methods are provided herein for assessment and determination of COVID-19 SARS-CoV-2 virus. The methods, systems and kits provided and exemplified herein enable population screening and surveillance in a rapid and low cost manner and with validated and dependable results.

The methods and approaches provided herein may be utilized, including with some modification, for isolation and analysis of other nucleic acid such as DNA, including DNA of an infectious agent, such as of a DNA virus, from saliva samples. In such instances, for example, the pH of the salt in the lysis/RNA stabilization buffer or the pH of the lysis/RNA stabilization buffer may be adjusted, particularly may be increased, particularly may be increased to be above pH7, to be more favorable for example to DNA isolation versus RNA isolation.

A saliva sample may be from a patient or individual having a disease or infection or at risk for or suspected of disease or infection. In some embodiments, the patient or individual obtains or collects the small volume sample. In some embodiments, the patient or individual is assisted by a non medical person in collection of the sample. In an embodiment, the sample is collected from a patient or individual by a non-medical person, such as a spouse, parent, friend, guardian, unrelated person, other employee, staff member, volunteer, etc that is not medically trained or involved in any medical profession. A small volume sample may be from a patient or individual having coronavirus infection, who was exposed to an individual having coronavirus infection, who is at risk for or suspected of coronavirus disease or infection, or may be from an individual for population or recurrent screening or surveillance as in the instance of employees, medical personnel, hospital or care workers, students, teachers, individuals who are or have been traveling. In some embodiments, the patient or individual obtains or collects their own sample. In embodiments, the sample is a saliva sample.

The invention provides a method for profiling and analysis of infectious agent RNA, particularly viral RNA, of saliva samples from one or more patient or individual comprising:
(a) obtaining one or more small volume saliva sample, wherein the sample is collected in or otherwise combined with a lysis/RNA stabilization buffer comprising guanidinine thiocyanate (GSCN), whereby infectious agent, particularly virus, is inactivated and RNA is stabilized;
(b) isolating RNA;
(c) reverse transcribing the RNA to generate cDNA;
(d) specifically amplifying any infectious agent RNA, particularly viral RNA, using primers and probes specific for the target infectious agent or virus; and
(e) determining the presence and identity of infectious agent RNA, particularly viral RNA, whereby primer amplified nucleic acid for the target infectious agent or virus is identified and characterized.

The invention provides a method for profiling and analysis of infectious agent RNA, particularly viral RNA, of saliva samples from one or more patient or individual comprising:
(a) obtaining one or more small volume saliva sample, wherein the sample is collected in or otherwise combined with a lysis/RNA stabilization buffer comprising guanidinine thiocyanate (GSCN), mild detergent and organic sodium salt whereby infectious agent, particularly virus, is inactivated and RNA is stabilized;
(b) isolating RNA;
(c) reverse transcribing the RNA to generate cDNA;
(d) specifically amplifying any infectious agent RNA, particularly viral RNA, using primers and probes specific for the target infectious agent or virus; and
(e) determining the presence and identity of infectious agent RNA, particularly viral RNA, whereby primer amplified nucleic acid for the target infectious agent or virus is identified and characterized.

In an embodiment, a method for analysis of coronavirus RNA in saliva samples from one or more patient or individual is provided. In an embodiment, a method for analysis of SARS-CoV-2 virus RNA in saliva samples from one or more patient or individual is provided.

The invention provides a method for profiling and analysis of infectious agent RNA, particularly viral RNA, of saliva samples from one or more patient or individual comprising:
(a) obtaining one or more small volume saliva sample, wherein the sample is collected in or otherwise combined with a lysis/RNA stabilization buffer comprising guanidinine thiocyanate (GSCN), mild detergent and organic sodium salt whereby infectious agent, particularly virus, is inactivated and RNA is stabilized;
(b) heating the saliva and lysis/RNA stabilization buffer sample briefly at about 65° C.;
(c) isolating RNA after brief heating in (b);
(d) reverse transcribing the RNA to generate cDNA;
(e) specifically amplifying any infectious agent RNA, particularly viral RNA, using primers and probes specific for the target infectious agent or virus; and
(f) determining the presence and identity of infectious agent RNA, particularly viral RNA, whereby primer amplified nucleic acid for the target infectious agent or virus is identified and characterized.

The invention provides a method for profiling and analysis of infectious agent RNA, particularly viral RNA, of saliva samples from one or more patient or individual comprising:
(a) obtaining one or more small volume saliva sample, wherein the sample is collected in or otherwise combined with a lysis/RNA stabilization buffer comprising guanidinine thiocyanate (GSCN), mild detergent and organic sodium salt whereby infectious agent, particularly virus, is inactivated and RNA is stabilized;
(b) heating the saliva and lysis/RNA stabilization buffer sample briefly for at about 65° C.;
(c) adding a protein denaturant or reducing agent to the heated sample;
(d) isolating RNA;
(e) reverse transcribing the RNA to generate cDNA;
(f) specifically amplifying any infectious agent RNA, particularly viral RNA, using primers and probes specific for the target infectious agent or virus; and
(g) determining the presence and identity of infectious agent RNA, particularly viral RNA, whereby primer amplified nucleic acid for the target infectious agent or virus is identified and characterized.

The invention provides methods for magnetic bead based purification or isolation of nucleic acid, particularly RNA, and including infectious agent nucleic acid or RNA, from one or more small volume sample, particularly one or more saliva sample. The invention provides a method of magnetic bead based purification of nucleic acid, particularly RNA, and including infectious agent nucleic acid or RNA, from one or more small volume sample, particularly one or more saliva sample, comprising:
(a) obtaining one or more small volume saliva sample, wherein the sample is collected in or otherwise combined with a lysis/RNA stabilization buffer comprising guanidinine thiocyanate (GSCN), mild detergent and organic sodium salt whereby infectious agent, particularly virus, is inactivated and RNA is stabilized;
(b) heating the saliva and lysis/RNA stabilization buffer sample briefly at about 65° C.;
(c) adding a binding buffer suitable for facilitating binding of nucleic acid, particularly RNA, to magnetic beads; and
(d) isolating RNA using magnetic beads.

The invention provides a method of magnetic bead based purification or isolation of nucleic acid, particularly RNA, and including infectious agent nucleic acid or RNA, from one or more small volume sample, particularly one or more saliva sample, comprising:
(a) obtaining one or more small volume saliva sample, wherein the sample is collected in or otherwise combined with a lysis/RNA stabilization buffer comprising guanidinine thiocyanate (GSCN), mild detergent and organic sodium salt whereby infectious agent, particularly virus, is inactivated and RNA is stabilized;
(b) heating the saliva and lysis/RNA stabilization buffer sample briefly for at about 65° C.;
(c) adding a protein denaturant or reducing agent to the heated sample;
(d) adding a binding buffer suitable for facilitating binding of nucleic acid, particularly RNA, to magnetic beads;
(e) isolating RNA using magnetic beads.

In further and additional embodiments of the method(s), once nucleic acid or RNA is isolated using magnetic bead based purification, the further steps may be taken or applied:
(a) reverse transcribing the RNA to generate cDNA;
(b) specifically amplifying any infectious agent RNA, particularly viral RNA, using primers and probes specific for the target infectious agent or virus; and
(c) determining the presence and identity of infectious agent RNA, particularly viral RNA, whereby primer amplified nucleic acid for the target infectious agent or virus is identified and characterized.

In an embodiment of the method(s), the saliva and lysis/RNA stabilization buffer sample is heated briefly at about 60-70° C., at about 60-75° C., at about 60-80° C., at about 65-70° C., at about 65° C. In an embodiment of the method, the saliva and lysis/RNA stabilization buffer sample is heated for 10-20 minutes, 10-15 minutes, about 15 minutes, about 15-20 minutes, about 15-18 minutes, about 15-17 minutes, 15 minutes.

In an embodiment, a protein denaturant which is a reducing agent is added prior to RNA isolation. In an embodiment a reducing agent is added prior to RNA isolation. In an embodiment, a reducing agent capable of breaking down protein disulfide bonds and stabilizing enzymes and proteins is added. In an embodiment, an agent selected from 2-mercaptoethanol, dithiothreitol (DTT), and TCEP (tris(2-carboxymethyl) is added. The protein denaturant or reducing agent is DTT.

In embodiments, reducing agent is added at about 15-20 μl amount per sample plus buffer volume of 400-600 μl. Reducing agent solution of 2-5M reducing agent, such as 2-5M DTT, about 3M DTT may be added. Final concentration of reducing agent such as DTT is on the order of 50-100 mM, 60-80 mM, about 70-80 mM. In an embodiment of the methods, heating the sample and further adding reducing agent/protein denaturant such as DTT serves to better liquefy the sample and thin the sample prior to RNA isolation, liquid handling etc. In an embodiment of the methods, heating the sample and adding reducing agent/protein denaturant such as DTT facilitates the sensitivity of the method and analysis for viral RNA, such that the saliva sample(s) can be handled and dependable and reproducible results can be obtained.

In some embodiments of the method(s), the saliva sample volume is less than 500 μl, about 200-400 μl, about 300 μl, about 200-300 μl, about 100-300 μl, about 150-300 μl, about 100-250 μl. In an embodiment, the sample volume is about 100-300 μl. In some embodiments of the method(s), the saliva sample volume is less than 300 μl, less than 200 μl, less than 100 μl, about 50-100 μl, less than 50 μl, provided that adequeate specificity, sensitivity and reproducibility can be achieved, including as provided herein and will be understood by one skilled in the art. In some embodiments of the method(s), saliva sample is combined with an approximately equal amount of lysis/RNA stabilization buffer. In some embodiments, the volume of lysis/RNA stabilization solution is less than 1 ml, about 500 μl or less, about 200-400 μl, about 300 μl or less, about 200-300 μl, about 300 μl, or about 250 μl. In an embodiment, the volume of lysis/RNA stabilization solution is about 300 μl or less, about 200-300 μl. Volume of lysis/RNA stabilization buffer is about 200-500 μl, 200-400 μl, about 300 μl, particularly for each saliva sample collected.

In some embodiments, the volume of lysis/RNA stabilization solution is less than 1 ml, about 500 μl or less, about 200-400 μl, about 300 μl or less, about 200-300 μl, about 300 μl, or about 250 μl. In an embodiment, the volume of lysis/RNA stabilization solution is about 300 μl or less, about 200-300 μl.

In important and relevant aspects and embodiments, the methods and approach provided herein is scalable for testing up to hundreds of saliva and RNA samples in a single assay. The success in scaling these two assays has permitted development and application to a new pooling strategy, described and exemplified herein for example in Example 8. This pooling strategy has three elements: it leverages the high sensitivity of the DRUL2 saliva assay; it uses pooling not of “specimen matrix” (saliva, or the more commonly pooled viral transport media containing RNA obtained by NPS), but of purified RNA; it uses an N-dimensional matrix strategy that can assay 1,000 samples in 30 PCR reactions.

In accordance with the method(s) of the invention, small volume sample(s) is collected and combined with a lysis/RNA stabilization solution. In some embodiments, the lysis/RNA stabilization solution is capable of lysing the virus and cells in the sample and of stabilizing RNA contained in the virus, cells or cell lysate of the sample. In embodiments, the lysis/RNA stabilization solution inactivates virus and stabilizes RNA. In embodiments, the sample and lysis/RNA stabilization solution are mixed, vortexed or shaken when combined. In some embodiments, the sample may be stored or left at room temperature for up to a few or several hours or up to a few or several days or one or more week prior to refrigeration or freezing. In some embodiments, the sample is then stored in refrigerated conditions, such as at about 40° F.; or about 4° C. for a brief time. In some embodiments, the sample is then stored in frozen sub-zero conditions, such as at about −70° C., and can be stored in such frozen conditions for months, numerous months, up to a year, at least a year. In some embodiments, after initial collection the sample is then stored in refrigerated conditions, such as at about 40° F. or about 4° C. for a brief time, up to a day or a few or several or many days. In some embodiments, the sample may be stored or left at room temperature for up to a few or several hours, up to 2 hours, up to 3 hours, up to 3 or 4 hours, up to a day, up to several days, for 2-6 days, for 3-5 days, up to a week, up to one or more weeks, prior to refrigeration or prior to freezing if being frozen rather than refrigerated. In some embodiments, the sample is then stored in refrigerated conditions, such as at about 40° F.; or about 4° C. for a brief time, up to a day or a few or several days. In some embodiments, the sample is stored in a freezer or in frozen temperature conditions, such as at about 30 or 32° F. or about 0° C., either after collection, after brief (2-4 hour) storage at room temperature, or after brief (1-2 day) refrigerated storage.

The saliva sample may be collected in an initial receptacle or vial and then transferred into a tube wherein the tube contains lysis/RNA stabilization solution and has a total volume capacity of 1.5 ml or less, 1.2 ml or less, or 1 ml or less. The saliva sample may be collected and then transferred into a tube wherein the tube or receptacle for receiving the saliva sample and containing lysis/RNA stabilization solution has a total volume capacity of 1.5 ml or less, 1.2 ml or less, or 1 ml or less. In an embodiment, the saliva sample is collected into a tube or wherein the tube or receptacle for receiving the small volume sample and containing lysis/RNA stabilization solution has a total volume capacity of 1.5 ml or less, such as a microtainer tube. Suitable sized tubes or containers are known and available in the art.

The purification/isolation method may be adapted for a may utilize a fully manual purification. In embodiments of manual purification centrifugation or a vacuum manifold, or a combination thereof, may be utilized, for example in order to pass solutions through columns. The purification/isolation method may be adapted for or may utilize Semi-automated purification. In embodiments of semi-automated purification, the lysis step and the precipitation or organic extraction step are carried out manually, while column purification is performed in an automated fashion, such as using an automated liquid handling system. Application of the isolation methods to fully automated purification is contemplated and an embodiment hereof, where all steps are performed using a fully automated system such as a fully equipped liquid handling system or a fully automated extraction system. Such fully automated systems are known and available in the art. In some embodiments, the fully automated systems are modified to adjust volumes, reagents, materials for small volume sample handling.

The methods herein may further comprise sequencing the RNA. RNA may be sequenced using any suitable or recognized method, steps, system(s) or kit(s), including manual, semi-automated or automated method(s), system(s) or kits. In some embodiments, kits such as Illumina TruSeq or Kapa Hyper Prep Kits are utilized.

As part of or commensurate with the methods herein, the isolated RNA may converted to cDNA. Methods for generating cDNA from RNA are well known and available to one skilled in the art. Any applicable and effective method should be suitable. The isolated RNA may be converted to cDNA for probing or specific primer applications, such as to assess expression or for sequencing of specific RNAs or gene products. In an embodiment, the isolated RNA may be converted to cDNA for sequencing of the viral genome or particularly for sequencing of aspects of specific coding regions or protein encoding portions thereof. In an embodiment, the isolated RNA, or cDNA derived therefrom, may be specifically amplified for direct sequencing of the viral genome or particularly for sequencing of aspects of specific coding regions or protein encoding portions thereof. In an embodiment, virus genome region(s) such as SARS-CoV-2 virus genome regions, which are associated with emerging, possible, or probable mutants or variants may be amplified and/or sequenced. In an embodiment, virus genome region(s) such as SARS-CoV-2 virus genome regions, including the S protein encoding region, associated with emerging, possible, or probable mutants or variants may be amplified and/or sequenced. In an embodiment, SARS-CoV-2 virus genome regions including the S mutations associated with emerging, possible, or probable mutants or variants may be amplified and/or sequenced. The isolated RNA may be converted to cDNA for cloning purposes, to be inserted or prepared in a vector, for introducing into or preparing a library therefrom. The cDNA may be sequenced, for instance using any of the art known methods, approaches or systems, including automated and high throughput direct sequencing systems and methods.

In an embodiment, virus genome region(s) such as SARS-CoV-2 virus genome regions, including the S protein encoding region, are specifically amplified for genome or region sequence. In an embodiment, cDNA is amplified using 2 primer sets (F1:CCAGATGATTTTACAGGCTGC (SEQ ID NO:35) and R1: CTACTGATGTCTTGGTCATAGAC (SEQ ID NO:36); F2:CTTGTTTTATTGCCACTAGTC (SEQ ID NO:37) and R1) and is then sequenced.

As part of or commensurate with the methods herein, the isolated RNA may amplified. In some embodiments, the RNA may be converted to cDNA and then amplified. Suitable methods and systems for amplification are known and available. For instance, methods, kits and systems for PCR amflication, including RT-PCR, wherein RNA is first reverse transcribed to cDNA and then amplifies are well known and available. Amplification methods and approaches may be useful particularly in the instances of small volume samples and/or where small amounts of RNA are being isolated. Another amplification approach, which is also useful for small volume or small quantity RNA samples, is loop-mediated isothermal amplification (LAMP). Combining LAMP with a reverse transcription step allows detection and evaluation of RNA. LAMP is carried out at a constant temperature (60-65° C.) and thus does not require a thermal cycler. LAMP methods may utilize Bst (Bacillus stearothermophilus) DNA polymerase.

In embodiments or the method, the patient or individual has a disease or infection or is at risk of or suspected of disease or infection. The infection may be a bacterial or viral infection. The infection may be with a known or unknown virus or bacteria. A viral infection or virus may be an influenza virus, a coronavirus, an unidentified virus, an RNA virus. A viral infection or virus may be an influenza virus, particularly influenza A and/or influenza B virus, a coronavirus, particularly SARS-CoV-2 virus, a respiratory RNA virus, one or more respiratory RNA virus, one or more seasonal respiratory RNA virus.

In an embodiment, the RNA stabilization buffer comprises guanidinine thiocyanate (GSCN), mild detergent and organic sodium salt and is prepared in nuclease free water. In an embodiment, the Lysis/RNA stabilization buffer comprises guanidinine thiocyanate (GSCN), mild detergent and sodium acetate (NaAc) and is prepared in nuclease free water. In an embodiment, the Lysis/RNA stabilization buffer comprises guanidinine thiocyanate (GSCN), sarkosyl and sodium acetate (NaAc) and is prepared in nuclease free water. In an embodiment, the Lysis/RNA stabilization buffer comprises ganidinine thiocyanate, sarkosyl and sodium acetate, ph between 5 and 6, and is prepared in nuclease free water. In an embodiment, the Lysis/RNA stabilization buffer has a concentration of components comprising GSCN in the range of 4-6M, particularly 5-6M; sarkosyl in a % range of 0.1%-0.5%, particularly 0.2%-0.6%, particularly 0.2% to 0.5%; sodium acetate (NaAc) at less than 200 mM, particularly 150 mM-200 mM, and at a pH of 5-6, particularly below pH 6, particularly pH 5.5. In an embodiment, the Lysis/RNA stabilization buffer has a concentration of components comprising 5M GSCN, 0.5% sarkosyl, 175 mM NaAc, pH 5.5. In an embodiment, the lysis/RNA stabilization buffer has a concentration of components comprising 5M GSCN, 0.2% sarkosyl, 150 mM NaAc, pH 5.5. In an embodiment, the lysis/RNA stabilization buffer has a concentration of components comprising 5M GSCN, 0.2% sarkosyl, 150 mM NaAc, pH 5.5 and further comprises an additional denaturing agent. In an embodiment, the lysis/RNA stabilization buffer has a concentration of components comprising 5M GSCN, 0.2% sarkosyl, 150 mM NaAc, pH 5.5 and further comprises dithiothreitol (DTT). In an embodiment, the lysis/RNA stabilization buffer has a concentration of components comprising 5M GSCN, 0.2% sarkosyl, 150 mM NaAc, pH 5.5 and further comprises 2-mercaptoethanol or beta-mercaptoethanol (BME). In an embodiment, the lysis/RNA stabilization buffer has a concentration of components comprising 5M GSCN, 0.5% sarkosyl, 125 mM NaAc, pH approximately 6, particularly pH 5.5. In an embodiment, the lysis/RNA stabilization buffer corresponds to DRUL buffer. In an embodiment, the lysis/RNA stabilization buffer corresponds to DRUL2 buffer. The GSCN concentration may be increased to 6M or decreased to 2.5M GSCN. In an embodiment the GSCN concentration is optimal at about 5M. The detergent, in a particular embodiment sarkosyl, may be suitable and used over a range up to 1%, particularly from 0.25% to 1%. The salt, particularly NaOAc, may be used over a range up to 300 mM. The range of NaOAc may be varied depending on the RNA isolation method, such as magnetic beads or another isolation method, and on the concentration and parameters of the other components in the buffer.

RNA isolation may utilize known and applicable methods for rapid and efficient isolation of RNA. In an embodiment, a rapid and highly scalable isolation method and/or system is utilize so as to facilityate rapid, cost effective screening of large numbers of samples in a short period of time and to enable rapid determination of results. In an embodiment, RNA is isolated using one or magnetic bead system and protocol. Exemplary magnetic bead systems includeBioMerieux NucliSENS® easyMag extraction system, Quiagen QuiaCube automated extraction system, KingFisher MagMAX or CleanNGS SPRI beads, for example as obtained from Bulldog Biosciences, and KingFisher Flex Magnetic Particle Processor and system. Amplification may utilize known and applicable methods for amplification such as polymerase chain reaction (PCR).

In particular embodiments of the present method and invention, RNA is isolated from saliva using the methods herein and in particular using magnetic beads for isolation. In accordance with embodiments of the invention, magnetic bead purification and the methods, system, buffers etc provided herein permit testing with a sensitivity that exceeds available tests, even including tests utilizing NPS samples for example.

In a particularly preferred embodiment of the invention, magnetic beads are utilized for RNA isolation. The invention includes a unique binding buffer and protocol for magnetic bead purification of nucleic acid, particularly and including RNA. In an embodiment, the binding buffer comprises PEG 8000, NaCl, Trisodium citrate, Tween 20, HCl. In an embodiment, the binding buffer may additionally include a protease, particularly proteinase K. In an embodiment, binding buffer includes PEG 8000 20% v/v, 2-3M or 2.5M NaCl, 1 mM Trisodium citrate, Tween 20 at about 0.05% v/v, or less than 0.1% v/v, HCl at 0.5-0.8 mM or about 0.5-0.6 mM or about 0.56 mM. In an embodiment, the binding buffer may additionally include proteinase K at about 100-200 μg/ml or 100-150 μg/ml or about 120 μg/ml, 121 μg/ml.

In an embodiment, the limit of detection of the present system and methods is better than or improved versus the alterative tests currently available including tests based on alternative sample types such as NPS or OP. In an embodiment, the limit of detection of the system and methods provided herein is less than 10 virus per μl of saliva. In an embodiment, the limit of detection of the present system and methods is 1 virus or less per μl of saliva. In an embodiment, the limit of detection of the system and methods provided herein is less than 1 virus per μl of saliva. In an embodiment, the limit of detection of the system and methods provided herein is 0.5 virus per μl of saliva.

In embodiments, the present system and methods provide faster testing and turnaraound time for test results and reduced costs per test versus other systems, particularly alterbative commercially available or approved systems, kits, methods for RNA virus, particularly coronavirus, particularly SARS-CoV-2 testing. The turnaround time and costs of testing are both less than alternative or approved tests such as those of Quest, Bioreference, Bioreliance, Cepheid, Yale for example. In particular embodiments, the turnaround time or time from sample collection to test results is significantly improved versus other available and current methods and systems. In particular embodiments, the cost per test is significantly reduced and is less than the cost of other available and currently approved methods and systems and kits, including as described herein and known to those skilled in the art, such as those of Quest, Bioreference, Bioreliance, Cepheid, Yale for example.

In accordance with the invention, methods and approaches are provided for pooling RNA samples for testing. In embodiments of these methods, RNA samples from up to 100 or more individuals can be combined or pooled for analysis. Multiplexing in alternative pools can further enable the identification of even a single positive among a hundred or even a thousand samples.

Attempts by others to developing pooling approaches to test multiple samples (dozens, hundreds, even thousands) at a time have been hampered by problems of sufficient RNA isolation for suitable specificity and sensitivity. Watkins and their team at Yale have investigated the potential of pooling saliva samples prior to RNA isolation and RT-qPCR amplification for viral RNA sequences. Pooling 5, 10 and 20 samples resulted in a reduction of sensitivity of 7.41%, 11.11% and 14.8% respectively. They conclude that as local outbreaks fluctuate, varying pool sizes in response will save resources. The group received the first EUA for pooled testing, but only up to 4 nasal swabs can be combined in a single test, making it most useful in high prevalence settings and rendering really large scale pooling or multitesting not feasible. They recommend considering the probability of false negatives in the content of the frequency of testing to design pooling or multitesting scenarios. For instance, in colleges and schools where students are tested weekly or every 14 days and the prevalence of infection is low, false negative results or low sensitivity could possibly be more tolerated as a negative one week might be a positive by the next testing cycle.

In accordance with the methods herein, additive sampling of subsets of pools of RNA samples leads to multiplicative power for precise detection, even of rare positives in large sample sets. Thus in accordance with embodiments of the methods herein, a positive sample can be specifically and precisely identified, for example without the need to repeat individual tests or test subsets to characterize and identified a positive in a pool.

In embodiments herein, up to 96 or about 100 samples are pooled and tested simultaneously. In embodiments herein, RNA isolated from up to 96 or about 100 samples are pooled and tested simultaneously. In embodiments herein, multiple pools of 96 or about 100 individuals are resampled in an additive strategy that gives multiplicative power. In embodiments herein, up to 960 or about 1000 samples are pooled and tested simultaneously. In embodiments herein, RNA isolated from up to 960 or about 1000 samples are pooled and tested simultaneously. In embodiments, RNA isolated from over a hundred or from hundreds of samples are pooled, and may be pooled in multiple or in distinct pool subsets, including so as to generate multiple or N dimensional pooling groups or sample sets, and tested simultaneously. In embodiments, RNA isolated from over a thousand or from thousands of samples are pooled, and may be pooled in multiple or in distinct pool subsets, including so as to generate multiple or N dimensional pooling groups or sample sets, and tested simultaneously.

Distinct from protocols and systems known and available wherein multiple samples themselves are combined and pooled for RNA isolation and analysis, including wherein multiple saliva samples or multiple NPS samples are combined or pooled, the present invention takes a distinct and unique approach. The present approach, and the various aspects thereof, permits analysis and testing of numerous samples, in this instance numerous saliva samples, readily 96 samples or more samples, at one time. In particular aspects and embodiments herein and particularly in the pooling methods and strategy provided herein, RNA is first isolated from saliva samples and then isolated RNA (not saliva in buffer) is pooled for analysis. This is distinct from other approaches wherein the ‘raw’ samples themselves or samples in buffer are combined and RNA is isolated from a combined sample. These other approaches suffer from problems of increased volume for RNA isolation and loss of sensitivity and specificity. In accordance with the present invention, after isolation of RNA from samples, particularly saliva sample(s), to generate isolated RNA corresponding to each original sample, aliquots of each RNA are combined, added to RNA binding buffer, combined with magnetic beads for further RNA isolation, and reconcentrated. This effectively concentrates the RNA and reduces the volume containing RNAs from multiple samples, dozens or even up to a hundred or hundreds of samples, even up to a thousand or thousands of samples. Pools of dozens, 12, 24, 48, 96, about 100, or even hundreds of isolated RNA samples can be combined and then concentrated in a reduced volume for further analysis of the pooled RNA sample.

In an embodiment, the invention provides a method and system for pooling and simultaneous analysis of dozens, hundreds of samples for RNA, particularly viral RNA, such as coronavirus RNA, wherein RNA is isolated from a small volume sample, such as a saliva sample, using a protocol or method that provides isolated RNA wherein less than 10 virus per μl of sample and on the order of 1 virus per μl of sample volume or even 0.5 virus per μl of sample can be sensitively identified in the isolated RNA with a target-directed primer amplification procedure. Thus, the quality of the isolated RNA is substantive and includes sufficient viral RNA for dependable and specific and sensitive analysis. The isolated RNA is then combined and pooled for further RNA isolation, RNA concentration and subsequent analysis. In accordance with the pooling method herein, RNAs are pooled, pooled RNAs are combined with a concentrated binding buffer (2× binding buffer), magnetic beads are added, RNA is isolated, and RNA is resuspended in a smaller than original pooled volume. Thus, in accordance with the pooling methods provided herein, isolated RNA is combined, re-isolated, and reconstituted in a reduced volume thereby concentrating the RNA.

In a particularly preferred embodiment of the invention, magnetic beads are utilized for RNA isolation, or as applicable in the pooling methods, RNA re-isolation or RNA concentration. The invention includes a unique binding buffer and protocol for magnetic bead purification of nucleic acid, particularly and including RNA, particularly for RNA isolation from pooled RNA samples. In this instance the binding buffer, denoted 2× binding buffer, comprises PEG 8000, NaCl, Trisodium citrate, Tween 20, HCl, wherein the amount of salt, particularly the concentration of NaCl and the concentration of trisodium citrate is effectively about twice the concentration as in the binding buffer utilized for RNA isolation, e.g. isolation with magnetic beads, for the original RNA isolation direct on the original sample, such as the original saliva sample RNA isolation. In an embodiment, the binding buffer may additionally and preferably include a protease, particularly proteinase K. In an embodiment, binding buffer includes PEG 8000 20% v/v, 4-6M, 4-5M or 4.5M NaCl, 2 mM Trisodium citrate, Tween 20 at about 0.05% v/v, or less than 0.1% v/v, HCl at 0.5-0.8 mM or about 0.5-0.6 mM or about 0.56 mM. In an embodiment, the binding buffer may additionally include proteinase K at about 100-200 μg/ml or 100-150 μg/ml or about 120 μg/ml, 121 μg/ml.

Primers may be directed to one or more specific infectious agent target sequence, particularly viral target sequence. One or more primers directed to one or more targets of infectious agent or viral RNA may be utilized. Forward and reverse primers are typically utilized to specifically amplify the sequence in the region between the primers. Exemplary primers are provided herein and serve as non-limiting examples. Suitable alternatives will be known and available to one skilled in the art. Design and evaluation of alternative primers, such as for example based on available public sequence of the target virus, such as available sequence of coronavirus, particularly SARS-CoV-2 coronavirus sequence, may be conducted by one skilled in the art.

Amplification may utilize a labeled probe, in an embodiment wherein the probe anneals to a specific probe target sequence located between the forward and reverse primers. In a particular embodiment, the probe has or incorporates a reporter or dye. During the extension phase of the amplification process, the 5′ nuclease activity of the DNA polymerase degrades the bound probe. This serves to release the reporter dye from a quencher dye and results in a signal, such as a fluorescent signal. The fluorescence intensity can be monitored and evaluated to determine the presence and/or amount of target sequence, which reflects the amount of amplified sequence and the presence and/or amount of infectious agent target sequence, particularly viral target sequence.

Each RNA target and its applicable primer set will preferably have a unique probe and reporter dye. This facilitates and enables multiple or multiplex testing and evaluation of multiple RNA targets simultaneously, such as in a single amplification, e.g. PCR, reaction. Thus, instead of amplifying separately for each distinct target, multiple targets are amplified simultaneously in a single reaction and can be assessed simultaneously. Suitable reporter dye(s) and quenchers or quencher dye(s) are known and available in the art. Examples include reporter dye FAM, Quasar 670HEX, LC610. Examples include quencher or quencher dye BHQ1, IABkFQ, BBQ.

In some embodiments, primers directed to multiple viral RNA target sequences are utilized. The target sequences may be directed to one or more, two or more, two, multiple specific viral RNA target sequences. Primers to a generic or housekeeping sequence present in any infectious agent RNA may be utilized. Suitable and applicable generic or housekeeping sequences present in any infectious agent RNA or viral RNA will be known and available to one skilled in the art. In one such embodiment, primers to RNAseP are utilized as a positive control for example for the presence of viral RNA. RNAseP primers suitable for use and application are known and exemplary suitable primers and probes are provided herein including in Table 1, Table 28 and Table 48. Exemplary RNAse primer and probe sets are provided in SEQ ID NOs: 10-12, 19-21 and 32-34 In an embodiment, primers are directed to viral RNA N protein, particularly N1 and N2 protein are utilized. N1 and N2 primers suitable for use and application are known and exemplary suitable primers and probes are provided herein including in Table 1, Table 28 and Table 48. Exemplary N1 and N2 primer and probe sets are provided in SEQ ID NOs: 1-3, 13-15 and 26-28 for N1 and SEQ ID NO:s:4-6, 16-18 and 29-31 for N2. N3 primers and probes may also be utilized, with exemplary primer probe sets provided in Table 1 and in SEQ ID NOs:7-9. In an embodiment, primers directed to viral RNA S protein sequence are utilized and may be utilized additionally or alternatively. In an embodiment, primers directed to coronavirus-specific N1, and/or N2, and/or S protein are utilized. In an embodiment, primers directed to coronavirus-specific N1, and N2 protein are utilized. In an embodiment, primers directed to coronavirus COVID-19 or SARS-CoV-2 virus-specific N1 and N2. In an embodiment a generic viral RNA sequence is used as a target, for example a sequence characteristic or all of a family of viruses, such as to all coronaviruses, or all influenza viruses or all severe acute respiratory syndrome (SARS)-related coronaviruses. In an embodiment, primer sequence directed to the RNA-dependent RNA polymerase (RdRp) gene of all severe acute respiratory syndrome (SARS)-related coronaviruses is utilized.

The primers utilized in the Examples provided herein are based on FDA approved primer sequences and correspond to those utilized in multiplex primer-probes used in the FDA-approved EUA protocol, Triplex CII-SARS-CoV-2 rRT-PCR test (Columbia University Laboratory of Personalized Genomic Medicine) (fda.gov/media/137983/download) These primers were generated using the following protocol as reported for the Triplex test: All available full-length SARS-CoV-2 genomic sequences were downloaded from GISAID on Apr. 3, 2020 (3210 sequences) and aligned to primer and probe sequences for N1 and N2 targets. Any genomic sequences containing ambiguous bases within primer or probe binding regions were excluded. 3153/3210 (98.22%) of SARS-CoV2 genomic sequences showed 100% nucleotide identity with N1 and N2 primers/probes. Fifty-six SARS-CoV-2 genomic sequences were not 100% identical to primer or probe sequences. There were no genomic sequences that showed greater than one nucleotide mismatch with any individual N1 or N2 primers and probes. One genomic sequence (hCoV19/Iceland/29/20201EPI_ISL_417618) had a single mismatch in forward N1 primer and a single mismatch in reverse N2 primer. All other genomic sequences had at most one mismatch against one of the N1 or N2 primers and probes, ensuring that at least one complete primer/probe set was 100% identical.

Exemplary and accepted assay primers include those provided herein, without limitation, including in Tables 1, 28 and 48. Alternative primers are also indicated herein and/or can be constructed by one skilled in the art based on available or known virus sequence, coronavirus sequence, Sars-CoV-2 sequence. For example, a reference SARS-CoV-2 virus genome sequence is provided in NC_045512.

Alternative coronavirus primers may be directed to different sequences on the same viral protein target sequence, or may be directed to alternative virus protein sequences. Alternative coronavirus primers may be generic to all coronaviruses, particularly all SARS-type coronaviruses. Primers may include primers directed to one or more virus, for example coronavirus, particularly SARS-CoV-2, and another prevalent or seasonal RNA virus, such as influenza virus. In this instance, an individual or patient presenting with symptoms can be assessed and simultaneously evaluated for coronavirus and influenze. If they are positive for coronavirus, they will be quarantined and treated appropriately. If they are positive for influenza, the treatment will be different and appropriate for influenza.

Exemplary other coronavirus sequence and primers are available and may be utilized to design primers or as primer alternatives. Chen J F et al (Lancet 2020; 395: 514-23; doi.org/10.1016/50140-6736(20)30154-9) describes assessment of coronavirus utilizing primers directed against all SARS-related coronavirus. These may be utilized to determine whether a coronavirus is responsible for symptoms for instance. Chen describes a set of such primers as the forward primer (5′-CAAGTGGGGTAAGGCTAGACTTT-3′) (SEQ ID NO:38) and the reverse primer (5′-ACTTAGGATAATCCCAACCCAT-3′) (SEQ ID NO:39) targeting 344 bp of RNA-dependent RNA polymerase (RdRp) gene of all severe acute respiratory syndrome (SARS)-related coronaviruses. In addition, Chen et al describes a primer set targeting the Spike (S) protein of the 2019-nCoV (COVID-19, SARS-CoV-2 virus): the forward primer (5′-CCTACTAAATTAAATGATCTCTGCTTTACT-3′) (SEQ ID NO:40) and the reverse primer (5′-CAAGCTATAACGCAGCCTGTA-3′) (SEQ ID NO:41) targeting the 158 bp of Spike (S) gene of this novel coronavirus.

Primer and probe sets for SARS-CoV-2 testing have also been reported and have been described as alternatives including those from the Chinese Center for Disease Control and Prevention (ivdc.chinacdc.cn/kyjz/202001/t20200121_211337.html), Charite Institute of Virology, Universitatsmedizin Berlin (Corman V M et al (2020) Euro Surveill 25(3):2000045) and Hong Kong University Chu DKW et al (2020) Clin Chem 6(4):549-555).

In an embodiment, the virus-specific primers, such as the N1 and/or N2 primers, may or can be adjusted or modified to ensure primer binding and recognition of virus mutants or variants. In an embodiment, the virus-specific primers, such as the N1 and/or N2 primers and/or S primers, may or can be adjusted or modified to ensure primer binding and recognition of virus mutants or variants. In an embodiment, virus-specific primers, such as S primers, may or can be added and incorporated in addition to N1 and N2 primers, and/or any such S primers may or can be adjusted or modified to ensure primer binding and recognition of virus mutants or variants. In any such instance wherein the virus, particularly coronavirus might or does mutate, such that one or more coronavirus variant may need to be screened for and identified, the primers may be adjusted or additional primers may be added so as to ensure that the virus and any and all variants are identified and amplified and can be screened.

SARS-CoV-2 virus emerging variants have been identified and increasingly have raised concern as they circulate in populations thoughout the world. A South African variant including three mutations (E484K, N501Y and K417N) in the ACE2 receptor binding domain of the S protein are included among these. Studies indicate that the South African variant B.1.351 might have acquired a partial resistance to neutralizing antibodies generated by natural infections or vaccinations (Stamatatos L et al. Science [Internet] 2021; dx.doi.org/10.1126/science.abg9175; Planas D et al. Nat Med [Internet]2021; dx.doi.org/10.1038/s41591-021-01318-5). A significant and impactful variant B.1.1.7 in the U.K. and variant B.1.526 in New York have also been identified and characterized as of concern and circulating in the general population.

The SARS-CoV-2 S (spike) protein binds to the host cell receptor and induces virus-cell membrane fusion, playing a key role in virus infection, and representing the target of COVID-19 vaccines. The SARS-CoV-2 S protein has been described functionally and by sequencing including in Huan et al (Huang Y et al Acta Pharmacologica Sinica (2020) 41:1141-1149; doi.org/10.1038/s41401-020-0485-4). The S protein includes an S1 subunit (140685 residues) and S2 subunit (residues 686-1273. The S1 subunit includes a receptor binding domain (RBD, residues 319-541). The S2 subunit includes the fusion peptide (FP, residues 788-806), heptapeptide repeat sequence 1 (HRI, 912-984), HR2 (residues 1183-1213), transmembrane domain (TM domain 1213-127) and cytoplasm domain (1237-1273) (Xia S et al Cell Mol Immunol 2020:17:765-7).

Previously vaccinated individuals have been identified as positive for SARS-CoV-2 post-vaccination using the saliva-based DRUL assay described and provided herein, as detailed in the examples. The assay was able to identify virus in saliva from these individuals using N1/N2 primer pairs. Isolated RNA from these patients was further amplified and genome sequence obtained to identify virus variants in these patients, including by amplification of genome region covering and including the S protein sequence. cDNA was PCR amplified using 2 primer sets (F1:CCAGATGATTTTACAGGCTGC (SEQ ID NO:35) and R1: CTACTGATGTCTTGGTCATAGAC (SEQ ID NO:36); F2:CTTGTTTTATTGCCACTAGTC (SEQ ID NO:37) and R1) and the PCR products were then sequenced.

In studies described herein Patient 1 virus variant mutations included the E484K mutation previously described and which confers resistance to a commonly elicited class of neutralizing antibodies (Wang Z et al Nature [Internet] 2021; dx.doi.org/10.1038/s41586-021-03324-6; Weisblum Y et al Elife [Internet] 2020; dx.doi.org/10.7554/eLife.61312) and D614G. Patient 2 also had the D614G mutation. Some of the Patient 1 substitutions (T95I, del144, E484K, A570D, D614G, P681H, D796H) were shared with B.1.526 NY variant (T95I, E484K, D614G7) and 3 were shared with Patient 2 (which had variants T95I, G142V and del144, F220I, R237K, R246T, D614G).

In embodiments of the invention, RNA isolated in accordance with the saliva based assay provided herein is further amplified or otherwise analyzed to determine, obtain, or otherwise identify variant SARS-CoV-2 virus or variant sequence(s). In an embodiment, specific primers directed to or against one or more variant sequence are included in a multiplex primer set. Primers may be generated by one skilled in the art based on the known variant sequence(s), including as provided herein. For example, a reference SARS-CoV-2 virus genome sequence is provided in NC_045512. S protein sequence is provided and referenced including as described above in Huang et al (2020).

Specific variant primers or primers encompassing known variant regions of the SARS-CoV-2 genome sequence may be utilized or added in combination with existing, standard or applicable N1 and N2 primers, such as the N1 and N2 primer pairs provided, described or utilized herein. In an embodiment, specific variant primers or primers encompassing known variant regions, such as including the S protein of SARS-CoV-2 or subunits or known variant sequence regions thereof, may be utilized or added in combination with existing, standard or applicable N1 and N2 primers. In an embodiment, isolated saliva sample RNA, including as obtained or isolated in accordance with the methods provided herein, is further evaluated by amplification with specific variant primer pairs. In an embodiment, one or more variant specific primer pair is provided and one or more primer is labeled or otherwise tagged for rapid and specific identification. In an embodiment, variant primers are designed and utilized which specifically, particularly, or only amplify variant sequence(s). In an embodiment, S protein or S protein region variant primers are utilized. The S protein or S protein region variant primers may specifically amplify the variant associated region of the genome, such as for follow on sequencing, or may specifically and only amplify variant sequence. The presence of variant sequence may be determined by any means or method known in the art, such as by sequencing, by specific variant amplification, by tagged or labeled primers or probes which specifically or particularly recognize variant sequence, etc.

The methods and systems are applicable to saliva-based screening of multiple viruses simultaneously or alternatively. Thus, screening for one or more coronavirus may be implemented using the methods. Screening and assessment of coronavirus, particularly SARS-CoV-2 virus, and also of influenza virus may be conducted in a single test or based on a single saliva sample.

Assays and methods provided herein may incorporate SARS-CoV-2 primers and probes for analysis and assessment of coronavirus and may simultaneously detect and determine the presence of other viruses, particularly one or more seasonal or prevalent virus in the population being screened or assessed. This can facilitate directed and specific treatment of any symptomatic, pre-symptomatic, or asymptomatic individual or patient. Thus, the method may be utilized to detect both SARS-CoV-2 and influenza A and/or B virus. The method may be utilized to detect both SARS-CoV-2, influenza A and/or B virus and Respiratory syncytial virus (RSV). Recently, Cepheid introduced a SARS-CoV-2, FluA, FluB and RSV combination test (XpertXpress SARS-CoV-2/Flu/RSV) to assess for seasonal respiratory viruses in a single test including coronavirus and that received EUA approval late September 2020. Notably, however, the Cepheid system and its GeneXpert automated test system uses and is designed and approved only for nasal pharyngeal and nasal swab samples and nasal wash aspirates. Also, utilizing modules or testing bays for each sample, the Cepheid smaller systems accommodate 2 or 4 modules, while the largest can test up to 80 samples at a time. Therefore, testing of large samples, or any pooling of samples is not an option or applicable with this system.

Primers suitable for the combination testing of SARS-CoV-2, influenza A, influenza B and RSV are described and known. In addition, or alternatively the methods for saliva testing may be adapted for alternative virus screening per se, utilizing only specific primers for another virus, particularly another respiratory RNA virus. Some suitable primers as well as dye probes directed to other viruses and applicable to alternative or combination testing, for example with coronavirus and SARS-CoV-2 primers and probes, are also provided below (adapted from KK To et al (2017) Emerg Microbes & Infect 6, e49; doi:10.1038/emi.2017.35).

Primers and Probes for Quantitative Reverse Transcription PCR of Other Viruses

| Virus | Primer/probe sequence (5′-3′) |
|---|---|
| Influenza A virus | Forward GACCRATCCTGTCACCTCTGAC (SEQ ID NO: 42) |
| | Reverse AGGGCATTYTGGACAAAKCGTCTA (SEQ ID NO: 43) |
| | Probe FAM-TGCAGTCCTCGCTCACTGGGCACG-BHQ1 (SEQ ID |
| | NO: 44) |
| Influenza B virus | Forward ACAATTGCCTACYTGCTTTCA (SEQ ID NO: 45) |
| | Reverse TCTTTCCCACCRAACCAAC (SEQ ID NO: 46) |
| | Probe HEX-AGAAGATGGAGAARGCAAAGCAGAACTAGC-IABKFQ |
| | (SEQ ID NO: 47) |
| Human metapneumovirus | Forward CATAYAARCATGCTATATTRAAAGAGTCTC (SEQ ID NO: 48) |
| | Reverse CCTATYTCWGCAGCATATTIGTAATCAG (SEQ ID NO: 49) |
| | Probe FAM-CAACHGCAGTRACACCYTCATCATTRCA-IABKFQ |
| | (SEQ ID NO: 50) |
| Respiratory syncytial virus | Forward CTTAGCAAAGTCAAGTTRAATGATACA (SEQ ID NO: 51) |
| | Reverse TGCACATCATAATTRGGAGTGTC (SEQ ID NO: 52) |
| | Probe HEX-ACYATYCAACGKAGYACAGGAGA-IABKFQ (SEQ ID |
| | NO: 53) |
| Parainfluenza virus type 1 | Forward GGAGGAGCAATTATACCTGGTCA (SEQ ID NO: 54) |
| | Reverse TGTATCCARTGAGTGGGCTA (SEQ ID NO: 55) |
| | Probe LC610-ATTAGGCCCGAGTGTRACRGATGATGC-BBQ (SEQ ID |
| | NO: 56) |
| Parainfluenza virus type 2 | Forward TATGCYATGGTGGGAGACATT (SEQ ID NO: 57) |
| | Reverse GCCATCTTGTTCCAAGTCCAT (SEQ ID NO: 58) |
| | Probe FAM-CCTCCCATTCCGCTGTGTTCAATRTACTT-IABKFQ |
| | (SEQ ID NO: 59) |
| Parainfluenza virus type 3 | Forward AGCTATYACTAGYATCTCAGGGT (SEQ ID NO: 60) |
| | Reverse CCCAATCTGATCCACTGTGT (SEQ ID NO: 61) |
| | Probe HEX-TCAGACAAGATGGAACAGTGCAGGCA-IABKFQ (SEQ |
| | ID NO: 62) |

A Multiplex PCR panel for respiratory viruses may be incorporated or further utilized as available commercially or previously described and known to the artisan. For example, the NxTAG Respiratory Pathogen Panel (IVD) (Luminex, Austin, Tex., USA) may be utilized or incorporated. This multiplex PCR for respiratory viruses detects respiratory viruses including influenza A virus, influenza B virus, RSV A and B, enterovirus/rhinovirus (EV/RV), parainfluenza viruses 1-4, hMPV, adenovirus, coronaviruses HKU1, NL63, 229E and OC43, and human bocavirus. In this assay, extracted nucleic acid can be added to pre-plated Lyophilized Bead Reagents, the reaction amplified via RT-PCR, and the reaction product used in bead hybridization. The hybridized and tagged beads are sorted and read, for example using a MAGPIX instrument, and the signals analyzed using a NxTAG Respiratory Pathogen Panel Assay File for the SYNCT Software (Luminex, Austin, Tex., USA).

Samples may be collected in several hour increments, twice a day, three or four times a day, every 4-6 hours, daily, every morning, every evening, every morning and evening, once a week, one a month, every two months, every four months, every six months, several times a year. Samples may be collected to evaluate the effects of a drug or agent, for example prior to and/or following administration of a drug or agent. Samples may be collected to evaluate the course of illness, for example by quantifying changes in viral load in saliva. These two uses may be combined, for example to evaluate the effects of a drug or agent as a function of viral load. In this instance, for example, one or more samples may be collected prior to, during, or after administration or treatment with a drug or agent, such as to determine the effect of a drug or agent on the virus, or the changes in viral load or virus RNA in saliva with administration of a drug or agent. In some embodiments, small volume samples may be collected or additionally collected at outset of symptom(s), such as one or more symptom or recognized parameter indicative of or associated with a disease or infection. Samples may be collected prior to and after or upon the recognition or development of one or more symptom or disease or infection parameter. Samples may be collected upon the development of a fever, cough, pain or discomfort, rash, etc.

The volume of saliva may be on the order of 100-300 μl, less than 300 μl, about 200 μl, less than 200 μl, less than 100 μl, 50 μl or less. The volume of saliva sample may be adjusted, including may be reduced, in coordination with the sensitivity and selectivity of the assay and method. Thus, in instances wherein the method can adequately and consistently detect virus in a saliva sample of reduced volume, such as less than 200 μl, less than 100 μl, 50 μl or less, sample saliva volume may be suitably reduced. IN embodiments, the volume of lysis/RNA stabilization solution is about equal to the volume of sample, particularly of saliva. In some embodiments of the system or kit, the volume of lysis/RNA stabilization solution is less than 1 ml, about 500 μl or less, about 300-400 μl, about 400 μl, about 300 μl or less, about 200-300 μl, or about 250 μl. In some embodiments of the system or kit, the volume of lysis/RNA stabilization solution is less than 300 μl, less than 200 μl, about 400 μl, about 300 μl or less, about 200-300 μl, or about 250 μl. In some embodiments of the system or kit, the volume of lysis/RNA stabilization solution may be on the order of 100-300 μl, less than 300 μl, about 200 μl, less than 200 μl, less than 100 μl, particularly as commensurate with the saliva sample volume for adequate testing results and sensitivity,

In some embodiments of the system or kit, the tube or receptacle for receiving the small volume sample and containing lysis/RNA stabilization solution has a total volume capacity of 1.5 ml or less, 1.2 ml or less, or 1 ml or less.

The invention further provides systems and kits for isolating and evaluating RNA in saliva samples, particularly in self-collected saliva samples, and for use and application of the methods herein. In an embodiment, the systems and kits are for use and application in serial monitoring of a population or of individuals for determining the presence of a virus and/or for detection of virus infection, even in pre-symptomatic and/or asymptomatic individuals. In an embodiment, the the systems and kits are for use and application for determining the presence of a coronavirus. In an embodiment, the the systems and kits are for use and application for determining the presence of COVID-19 virus SARS-CoV-2.

In embodiments, a system or kit is provided for RNA isolation and analysis of small volume saliva samples from a patient or individual comprising:
(a) a means for self-collection of a saliva sample by the patient or individual or by a non medical person, comprising a receptable for a spit or aspirate;
(b) a tube or receptacle for receiving the small volume sample on collection and containing a volume of lysis/RNA stabilization solution whereby cells in the sample are lysed and RNA is stabilized; and
(c) one or more appropriate label(s) for designating the name or identity of the patient or individual, date of sample collection and time of sample collection.

In an embodiment, the system or kit is provided for RNA isolation and analysis of small volume saliva samples and determination of the presence of viral RNA in a patient or individual. In an embodiment, the system or kit is provided for RNA isolation and analysis of small volume saliva samples and determination of the presence of virus infection in a patient or individual. In an embodiment, the system or kit is provided for RNA isolation and analysis of small volume saliva samples and determination of the presence of coronavirus infection in a patient or individual. In an embodiment, the system or kit is provided for RNA isolation and analysis of small volume saliva samples and determination of the presence of COVID-19 infection or SARS-CoV-2 virus infection in a patient or individual. In an embodiment, the system or kit is provided for RNA isolation and analysis of small volume saliva samples and determination of the presence of COVID-19 infection or SARS-CoV-2 virus infection and/or another virus infection in a patient or individual. In an embodiment, the system or kit is provided for RNA isolation and analysis of small volume saliva samples and determination of the presence of COVID-19 infection or SARS-CoV-2 virus infection and/or influenza virus infection in a patient or individual.

In an embodiment, the tube or receptacle for receiving the small volume saliva sample on collection contains a volume of lysis/RNA stabilization solution whereby cells in the sample are lysed, RNA is stabilized and virus is inactivated.

In embodiments, a system or kit is provided for RNA isolation and analysis of small volume saliva samples from a patient or individual comprising:
(a) a means for self-collection of a saliva sample by the patient or individual or by a non medical person, comprising a receptable for a spit or aspirate;
(b) a tube or receptacle for receiving the small volume sample on collection and containing a volume of lysis/RNA stabilization solution whereby RNA is stabilized and virus is inactivated; and
(c) one or more appropriate label(s) for designating the name or identity of the patient or individual, date of sample collection and time of sample collection.

In embodiments, the system or kit may further comprise an envelope or mailing container for shipment of the sample to a laboratory or facility for RNA isolation and analysis.

In some embodiments, the system or kit may be for RNA analysis and virus infection determination of multiple small volume saliva samples collected in series from a patient or individual over days, weeks or months or from numerous patients or individuals in a single day or over several days or over a week comprising:
(a) a set of numerous means for self-collection of individual small volume saliva samples by the patient or individual or by a non-medical person, each comprising a receptable for a spit or aspirate;
(b) a set of numerous tubes or receptacles each individually for receiving a small volume saliva sample on collection and containing a volume of lysis/RNA stabilization solution whereby RNA is stabilized and virus is inactivated;
(c) numerous appropriate label(s) for designating the name or identity of the patient or individual, date of sample collection and time of sample collection; and
(d) numerous envelopes or mailing containers for shipment of each sample or several samples to a laboratory or facility for RNA isolation and analysis.

In some embodiments of the system or kit, the volume of lysis/RNA stabilization solution is less than 1 ml, about 500 μl or less, about 300 μl, about 300 μl or less, about 200-300 μl, or about 250 μl. In an embodiment, the volume of lysis/RNA stabilization solution is about 300 μl, about 300 μl or less, about 200-300 μl, or about 250 μl.

In some embodiments of the system or kit, the tube or receptacle for receiving the small volume sample and containing lysis/RNA stabilization solution has a total volume capacity of 1.5 ml or less, 1.2 ml or less, or 1 ml or less.

In the specification, numerous specific details are set forth in order to provide a thorough understanding of the present embodiments. It will be apparent, however, to one having ordinary skill in the art that the specific detail need not be employed to practice the present embodiments. In other instances, well-known materials or methods have not been described in detail in order to avoid obscuring the present embodiments.

Throughout this specification, quantities are defined by ranges, and by lower and upper boundaries of ranges. Each lower boundary can be combined with each upper boundary to define a range. The lower and upper boundaries should each be taken as a separate element.

Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such non-limiting examples and illustrations includes, but is not limited to: “for example,” “for instance,” “e.g.,” and “in one embodiment.”

In this specification, groups of various parameters containing multiple members are described. Within a group of parameters, each member may be combined with any one or more of the other members to make additional sub-groups. For example, if the members of a group are a, b, c, d, and e, additional sub-groups specifically contemplated include any one, two, three, or four of the members, e.g., a and c; a, d, and e; b, c, d, and e; etc.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### Example 1

### Testing for Coronavirus

The recent outbreak of new coronavirus SARS-COV2 and the COVID-19 pandemic underscores an imminent need for a system, method and approach ad provided herein. At the outset of the appearance of COVID-19, particularly in the U.S., there was a lack of reliable and available tests to assess whether an individual was infected. Although testing has improved, there remains a backlog of patients and individuals needing or wanting testing, even with drive up testing centers opened in many states and locations. The possibility of testing all or most Americans, which would provide knowledge of the valid number of infected individuals, even those that are asymptomatic or presymptomatic, and reduce ongoing transmission, while a reasonable and scientifically important goal, is not presently feasible. More broad-based testing would permit case detection and isolation and determination of the prevalence and contagiousness of undocumented SARS-COV2 infections. In the absence of broad-based testing, and the general unavailability of self-testing or self-collection options, individuals are left to follow stay at home guidelines and wait until the number of infections starts dropping.

The CDC's testing guidelines for COVID-19 suspected patients as of April 2020 was limited to invasive sampling and swabs: Nasopharyngeal (NP) swabs can be used for testing asymptomatic persons in a healthcare setting, including long term care facilities. At this time anterior nares and mid-turbinate specimen collection are only appropriate for symptomatic patients and both nares should be swabbed. The guidance below addresses options for self-collection of specimens once a clinical determination has been made to pursue COVID-19 testing.

For initial diagnostic testing for COVID-19, CDC recommended collecting and testing an upper respiratory specimen. Nasopharyngeal specimen is the preferred choice for swab-based SARS-CoV-2 testing. When collection of a nasopharyngeal swab is not possible, acceptable alternatives were: An oropharyngeal (OP) specimen collected by a healthcare professional, or; A nasal mid-turbinate (NMT) swab collected by a healthcare professional or by onsite self-collection (using a flocked tapered swab), or An anterior nares (nasal swab; NS) specimen collected by a healthcare professional or by onsite self-collection (using a flocked or spun polyester swab); or Nasopharyngeal wash/aspirate or nasal aspirate (NA) specimen collected by a healthcare professional.

As indicated above, the CDC guidelines (as of April 2020) are largely geared to collections by a health care professional or onsite self-collection (thus in the presence of a health care professional at a testing center). Collection of such samples puts health care workers at risk and is unduly invasive and difficult for patients and individuals already suffering or in stressful and demanding situations and conditions.

In order to be able to test a greater portion of the population and to validly determine and assess infection, even in asymptomatic or pre-symptomatic individuals, a true self-collection system is warranted and indeed necessary. We have undertaken to adapt the system and approach provided herein to self-collection of alternative samples, including from saliva samples and swabs.

### New System and Kit for Isolation and Evaluation of RNA from Saliva Samples

RNA kits were generated and assessed for processing saliva samples.

Five reagents were evaluated for SARS-CoV-2 testing in the home.
The Five buffers tested are:
1. Home-made (DRUL (Darnell-Rockefeller University Lab) buffer): 5M GSCN (Guanidinium thiocyanate)/0.5% Sarkosyl/25 mM sodium acetate pH 6 (RBD modifications Chomczynski & Sacchi protocol, based in part on other viral literature using up to 6M GSCN, need to keep pH buffered, on low end for RNA stability.
2. PRIMESTORE MTM (Thomas scientific)
3. Buffer AVL (Qiagen)
4. TRIzol (Thermo Fisher)
5. Second generation Home-made, for use with magnetic bead purification: 300 ul (DRUL2 buffer)=5M GSCN, 0.2% Sarkosyl, 140 mM DTT, 150 mM NaOAc pH 5.5;
   a. dilute 1:1 with 300 ul saliva, mix, transport to lab; final concentrations after mixing saliva are 2.5M GSCN, 0.1% Sarkosyl, 70 mM DTT, 75 mM NaOAc pH 5.5
   b. add magnetic bead binding buffer: PEG 8000 20% (w/v)
      Sodium chloride 2.5 M
      Trisodium citrate 1 mM
      Tween 20 0.05% (v/v)
      HCl 0.56 mM
   c. add magnetic beads; 10 ul MagMax (ThermoFisher), mix 4-5 minutes or 100 ul SPRI Magnetic Beads (Bulldog Biosciences)
   d. purify RNA with magnet to remove RNA from DRUL2 and binding buffer
   3. wash 2× in 80% ETOH, using magnet to isolate beads after washing (3 min each wash with mixing)
   4. elute in 20-50 ul ddH2O, or in 10 mM Tris pH 7.5
   5. add 5 ul-10 ul into 25 ul total RT-PCR reaction, using standard reagents

Individuals were given tubes with buffer and a dropper. They were instructed to spit into a cup from their home, use the dropper to take a pre-determined amount out and put it in the barcoded tube with buffer. Initial experiments utilized 300 ul volume of saliva. Further experiments utilized 150 ul to 300 ul. For “stabilizer”, binding buffer above in method 5 was ultized.

### Clinical Genomics SARS CoV-2 Assay

Intended Use: The Rockefeller Clinical Genomics SARS CoV-2 assay is a qualitative real-time reverse transcriptase polymerase chain reaction (RT-PCR) test for the qualitative detection of RNA from the SARS-CoV-2 in patient saliva. The test is intended to be used for detection of viral RNA in saliva specimens collected in a transport medium that potentially inactivates the virus while stabilizing the viral RNA. Saliva specimens can be either self-collected or collected at the physician's office and returned (e.g. to the Rockefeller University Clinical Genomics Laboratory (RU CGL)) for testing. The test uses a stringent extraction protocol that provides for a highly sensitive detection of SARS CoV-2 using primers and probes from the CDC EUA with real-time PCR methodology. The assay was validated as per the Wadsworth Validation Procedure for SARS-CoV-2 LDT.

Summary and Explanation of the Test: The RU CGL SARS CoV-2 assay is a real-time PCR assay using primers and probe sets to detect RNA from SARS CoV-2. The test will assay saliva samples self-collected at home, in a physician's office, or elsewhere and delivered or shipped to the RU CGL. Principles of the Procedure: Saliva samples are collected at home, in a physician's office or elsewhere and added to buffer at the site of collection, stabilizing RNA and potentially inactivating the virus at the point of collection. The extraction reagents and method used here is a modified version of the guanidinium thiocyanate-phenol-chloroform extraction method, used in molecular biology for denaturing protein from a variety of sources and subsequently isolating RNA. This method has been shown to produce higher purity and recovery of RNA as compared to column-based systems. Furthermore, these reagents are widely available and economical, and variations of the method that include guanidinium thiocyanate are used in many commercial kits, including those approved for use in SARS-CoV-2 RT-PCR (for example Qiagen buffer AVL). After samples are transported to the RU CGL, RNA is purified and reverse transcribed into cDNA. cDNA is amplified using primers and probes (N1/N2 and RNaseP as a positive control) that have been validated by the CDC and described in their EUA. In the PCR assay, the probe is hydrolyzed during primer amplification of the primer specific region, cleaving and releasing the fluorophore from the quencher, resulting in a fluorescence signal proportional to the target sequence present in the sample. The fluorescence intensity is measured using the Bio-Rad CFX96 Touch Real-Time PCR Detection System.

CDC designated primers and probes are provided below in TABLE 1:

TABLE 1
PCR PRIMERS AND PROBES
2019-Novel Coronavirus (2019-n-CoV) Real Time RT-PCR Panel Primers and Probes

| | | | | Working |
|---|---|---|---|---|
| Name | Description | Oligonucleotide Sequence | Label | Conc. |
| 2019-nCoV_N1- | 2019-nCoV_N1 | 5′-GACCCCAAAATCAGCGAAAT-3′ | None | 20 μM |
| F | Forward Primer | (SEQ ID NO: 1) | | |
| 2019-nCoV_N1- | 2019-nCoV_N1 | 5′-TCTGGTTACTGCCAGTTGATTCTG-3′ | None | 20 μM |
| R | Reverse Primer | (SEQ ID NO: 2) | | |
| 2019-nCoV_N1- | 2019-nCoV_N1 | 5′-FAM- | FAM, BHQ-1 | 5 μM |
| P | Probe | ACCCCGCATTACGTTTGGTGGACC-BHQ-1- | | |
| | | 3′ (SEQ ID NO: 3) | | |
| 2019-nCoV_N2- | 2019-nCoV_N2 | 5′-TTACAAACATTGGCCGCAAA-3′ | None | 20 μM |
| F | Forward Primer | (SEQ ID NO: 4) | | |
| 2019-nCoV_N2- | 2019-nCoV_N2 | 5′-GCGCGACATTCCGAAGAA-3′ | None | 20 μM |
| R | Reverse Primer | (SEQ ID NO: 5) | | |
| 2019-nCoV_N2- | 2019-nCoV_N2 | 5′-FAM- | FAM, BHQ-1 | 5 μM |
| P | Probe | ACAATTTGCCCCCAGCGCTTCAG-BHQ-1- | | |
| | | 3′ (SEQ ID NO: 6) | | |
| 2019-nCoV_N3- | 2019-nCoV_N3 | 5′-GGGAGCCTTGAATACACCAAAA-3′ | None | 20 μM |
| F | Forward Primer | (SEQ ID NO: 7) | | |
| 2019-nCoV_N3- | 2019-nCoV_N3 | 5′-TGTAGCACGATTGCAGCATTG-3′ | None | 20 μM |
| R | Reverse Primer | (SEQ ID NO: 8) | | |
| 2019-nCoV_N3- | 2019-nCoV_N3 | 5′-FAM- | FAM, BHQ-1 | 5 μM |
| P | Probe | AYCACATTGGCACCCGCAATCCTG-BHQ-1- | | |
| | | 3′ (SEQ ID NO: 9) | | |
| RP-F | Rnase P | 5′-AGATTTGGACCTGCGAGC-3′ | None | 20 μM |
| | Forward Primer | (SEQ ID NO: 10) | | |
| RP-R | Rnase P | 5′-GAGCGGCTGTCTCCACAA-3′ | None | 20 μM |
| | Reverse Primer | (SEQ ID NO: 11) | | |
| RP-P | Rnase P Probe | 5′-FAM- | FAM, BHQ-1 | 5 μM |
| | | TTCTGACCTGAAGGCTCTGCGCG-BHQ-1- | | |
| | | 3′ (SEQ ID NO: 12) | | |

Reagents: Patients are mailed or otherwise provided a Sample Collection Kit. A component of the kit is a vial that contains a pre-measured volume of guanidine thiocyanate, sarkosyl and sodium acetate buffer (Darnell RU Lab buffer denoted DRUL). The DRUL buffer will denature proteins, stabilize RNA and may also inactivate virus. Patients are instructed to add saliva to this buffer. DRUL buffer contains the following reagents:

| Reagent | Purpose | Manufacturer | Catalog # | Storage |
|---|---|---|---|---|
| Guanidine | Protein denaturing | Fisher BioReagents | BP221-1 | Room |
| Thiocyanate | agent, Isolation of RNA | | | temp |
| Sarkosyl | Cell lysis, detergent | Fisher BioReagents | BP234-500 | Room |
| | | | | temp |
| Sodium Acetate (3M), | Precipitation of RNA | Invitrogen | AM9740 | Room |
| pH 5.5, RNase free | | | | temp |
| Nuclease-free Water | Dilution | Ambion | AM9932 | Room |
| | | | | temp |

Analytical Validation/Performance Characteristics: Analytical Validation was performed using saliva as the matrix to address accuracy and precision:
(A) The Limit of Detection (LoD) was established by testing, in triplicate, 10-fold serial dilutions (200→20→2→0.2→0.02 copies/ul) of quantified whole viral genomic RNA (SARS-Related Coronavirus 2—BEI Resources), spiked into pooled specimen matrix. Our data showed that the virus was detected at 1 copy/uL.
(B) The precision of the LoD of 1 copy/uL was confirmed using pooled saliva samples at concentrations of 24140.2 copies/uL, run in replicates of 20. The assay was precise for detection of CoV-2 at 1 copy/uL
(C) Verification of the LoD was further confirmed with 20 individual saliva samples at concentrations of 24140.2 copies/uL, run in triplicates. The results as determined by ct values and standard deviation were accurate and precise at the LoD of 1 copy/uL.

Clinical Evaluation: Clinical Evaluation with synthetic RNA spiked into five different individual specimen matrices at 2×, 4×, 6×, 8×, 10×, and 100× the LoD (1 copy/ul) was performed. In addition, 10 negative specimen matrices were assayed. The results obtained confirmed the performance of the assay.

### Example 2

### Saliva Testing for Sars-Cov-2

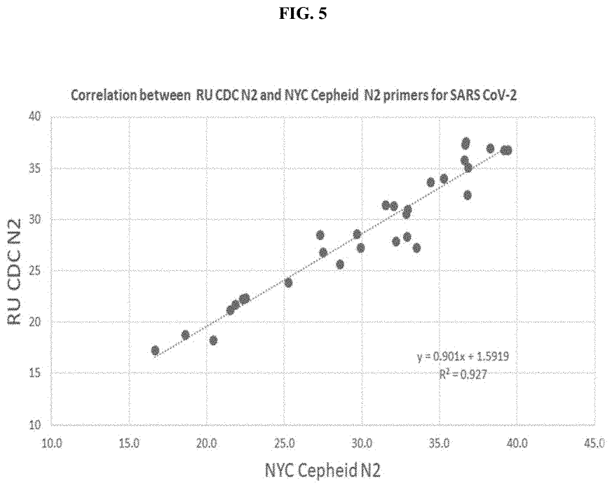

Components and aspects of a new set of protocols are presented to validate saliva testing using home-collected samples with a very sensitive, safe, inexpensive and scalable strategy. In a first section particularly in the present Example 2, Validation of SARS-CoV-2 LDT, describes the development of a guanidinium thiocyanate-detergent solution (lysis buffer) that inactivates SARS-CoV in saliva on contact, and is successful in subsequent extraction assays to be able to detect SARS-CoV-2 RNA with limits of detection and sensitivity that is very favorable and comparable to that used in NYCDOH-EUA established (Cepheid) tests (FIG. 5).

In a second section provided in Example 3, SARS-CoV-2 Saliva Testing with DRUL2 at Scale, the use of guanidinium thiocyanate-detergent lysis buffer is described in a downstream protocol that has been designed to detect saliva in a safe and scalable assay, overcoming many traditional hurdles in the use of saliva for SARS-CoV-2. Testing saliva, which is easy to obtain, has been held back because it has been challenging to reliably and technically detect viral RNA from this specimen, in contrast to nasopharyngeal swabs, the original FDA standard, which are difficult to obtain but, when placed in a viral culture media, have been easy to assay. We have developed a strategy to bridge these challenges by effectively and consistently extracting RNA from saliva in a safe manner with an extremely sensitive assay. This protocol utilizes magnetic beads for RNA purification and increases the sensitivity to be improved and better than other tests presently available and in use.

The net of these two sections and validated assays has allowed us, under the auspices of two NYS EUA validated tests (the “Rockefeller Clinical Genomics Laboratory SARS CoV-2 assay” described in the first section and this Example 2 and the “DRUL2 Rockefeller Clinical Genomics Laboratory SARS CoV-2 assay (RUCGL SCV)” described in an additional section and in Example 3, to undertake diagnostic assays for SARS-CoV-2 in over 14,000 individuals, reporting all positive and negative results to individuals and to NYS according to State guidelines.

The success in scaling these two assays has allowed us to develop a new pooling strategy, described in a third section and Example 10, Pooling Strategy for SARS-CoV-2 Saliva Testing with DRUL2. This strategy has three elements: it leverages the high sensitivity of the DRUL2 saliva assay described above; it uses pooling not of “specimen matrix” (saliva, or the more commonly pooled viral transport media containing RNA obtained by NPS), but of purified RNA; it uses an N-dimensional matrix strategy that can assay 1,000 samples in 30 PCR reactions.

### Validation of Sars-Cov-2 Ldt

### Rockefeller Clinical Genomics Laboratory SARS CoV-2 Assay

Intended Use: The Rockefeller Clinical Genomics SARS CoV-2 assay is a real-time reverse transcriptase polymerase chain reaction (RT-PCR) test for the qualitative detection of RNA from the SARS-CoV-2 in patient saliva. The test is intended to be used for detection of viral RNA in saliva specimens collected in a transport medium that potentially inactivates the virus while stabilizing the viral RNA. Saliva specimens can be either self-collected or collected at the physician's office and returned to the Rockefeller University Clinical Genomics Laboratory (RU CGL) for testing. The test uses a stringent extraction protocol that provides for a highly sensitive detection of SARS CoV-2 using primers and probes from the CDC EUA with real-time PCR methodology. The assay was validated as per the Wadsworth Validation Procedure for SARS-CoV-2 LDT.

Summary and Explanation of the Test: The Rockefeller University Clinical Genomics Laboratory (RU CGL) SARS CoV-2 assay is a real-time PCR assay using primers and probe sets to detect RNA from SARS CoV-2. The test uses saliva samples self-collected at home, in a physician's office, or elsewhere and delivered or shipped to the RU CGL.

Principles of the Procedure: Saliva samples are collected at home, in a physician's office or elsewhere and added to buffer at the site of collection, stabilizing RNA and potentially inactivating the virus at the point of collection. The extraction reagents and method used here is a modified version of the guanidinium thiocyanate-phenol-chloroform extraction method,[1] widely used in molecular biology for denaturing protein from a variety of sources and subsequently isolating RNA. This method has been shown to produce higher purity and recovery of RNA as compared to column-based systems.[2] Furthermore, these reagents are widely available and economic, and variations of the method that include guanidinium thiocyanate are used in many commercial kits. After samples are transported to the RU CGL, RNA is purified using either standard molecular biology methods or a column-based extraction kit used in the CDC 2019-Novel Coronavirus (2019-nCoV) Real-Time RT-PCR Diagnostic Panel EUA (Qiagen QIAmp DSP Viral RNA Mini Kit) and reverse transcribed into cDNA. cDNA is amplified using primers and probes that have been validated by the CDC and described in their EUA. In the PCR assay, the probe is hydrolyzed during primer amplification of the primer specific region, cleaving and releasing the fluorophore from the quencher, resulting in a fluorescence signal proportional to the target sequence present in the sample. The fluorescence intensity is measured using the Bio-Rad CFX96 Touch Real-Time PCR Detection System.

Reagents Used: Sample Collection Kits (described below in Sample Collection) are either mailed to the patient or given to them by a healthcare providers on request. A component of the kit is a vial containing one of two buffers, the Darnell Rockefeller University Lab (DRUL) buffer or PrimeStore MTM (Longhorn Vaccines and Diagnostics LLC). The DRUL buffer contains guanidine thiocyanate, sarkosyl, and sodium acetate (TABLE 2). Both buffers will denature proteins, stabilize RNA and may also inactivate virus. Patients are instructed to add saliva to this buffer.

The DRUL buffer contains the following reagents:

TABLE 2
DRUL buffer

| Reagent | Purpose | Manufacturer | Catalog # | Storage |
|---|---|---|---|---|
| Guanidine | Protein denaturing agent, | Fisher | BP221-1 | Room |
| Thiocyanate | Isolation of RNA | BioReagents | | temp |
| Sarkosyl | Cell lysis, detergent | Fisher | BP234-500 | Room |
| | | BioReagents | | temp |
| Sodium Acetate (3M), | Precipitation of RNA | Invitrogen | AM9740 | Room |
| pH 5.5, RNase free | | | | temp |
| Nuclease-free Water | Dilution | Ambion | AM9932 | Room |
| | | | | temp |

Samples received in either DRUL buffer or PrimeStore MTM are extracted using either the phenol extraction method (Option 1) or a column-based method (Option 2). Extracted RNA is then used in a PCR reaction.

Reagents Used in RNA Extraction:

### Option 1: Phenol Extraction

TABLE 3
Phenol Extraction Reagents.

| Reagent | Manufacturer | Catalog # | Storage |
|---|---|---|---|
| Acid-Phenol:Chloroform, pH | Ambion | 9720 | Room |
| 4.5 (with IAA, 125:24:1) | | | temp |
| OmniPur Chloroform:Iso- | Calbiochem | 3155- | Room |
| Amyl Alcohol 24:1 | | 100 ml | temp |
| Isopropanol, RNase and | Acros | 327270010 | Room |
| Protease-free | Organics | | temp |
| GlycoBlue coprecipatant | Invitrogen | AM9515, | −20□ C. |
| Ethanol 200 proof anhydrous | Decon Labs | 2716 | Room |
| | | | temp |

### Option 2: Column-Based Extraction

TABLE 4
Column-based Extraction Reagents.

| Reagent | Manufacturer | Catalog # | Storage |
|---|---|---|---|
| QIAmp DSP Viral RNA Mini | Qiagen | 61904 | Room temp |
| Kit | | | |

Reagents used in PCR:

TABLE 5
PCR Reagents.

| Reagent | Manufacturer | Catalog # | Storage |
|---|---|---|---|
| TaqPath 1 Step RT-PCR | Life Tech | A15300 | −20□ C. |
| 2019-nCoV CDC EUA Kit | IDT | 10006606 | −20□ C. |
| (CDC validated lots only) | | | |
| Quantitative synthetic RNA | BEI Resources | NR-52358 | −60□ C. |
| from SARS-Related Coronavirus | | | or colder |
| 2 (2.9 × 10⁸ genomic | | | |
| equivalents/ml) | | | |
| Nuclease-free water | Ambion | AM9932 | Room |
| | | | temp |

Equipment and Materials Used
Bio-Rad CFX96 Touch Real-Time PCR Detection System
Eppendorf microcentrifuge 5424
Eppendorf centrifuge 5910 R (refrigerated)
Pipettes P10, P20, P200
Aerosol barrier pipette tips (DNase/RNase free)
Non-stick RNase-free 1.5 ml microfuge tubes
0.2 ml PCR reaction plates (96 well plates)
Microseal B PCR plate seals
Disposal gloves, gowns, masks

Warnings and Precautions
All samples should be handled with universal precautions as potentially infectious and handled within biological safety cabinets of Class 2 or higher.
Use personal protective equipment when handling samples and reagents.
Take care not to introduce contamination with amplicons from previous reactions.
   Use separate spaces for extraction and amplification.
   Use dedicated equipment and personnel protective equipment in those rooms.
   Always use barrier pipette tips.
   Change gloves between specimens.
   Keep reagents capped or covered whenever possible.
   Clean up spills immediately.
   Use RNase Away to decontaminate pipettes, work surfaces, and centrifuges to minimize risk of contamination.
   Dispose of chemical or biological waste according to institutional procedures.

Reagent Storage, Handling, and Stability
Use storage temperatures listed above.
For reagents without manufacturer specified expiration dates, the RU CGL uses the date one year after receipt as the expiration date.
Positive control RNA should be aliquoted to avoid freeze thaw cycles.

Specimen Collection, Handling, and Storage

Specimen Collection Procedure—Patients will collect saliva at home or other locations such as a physician's office, add approximately 300 ul (via an approximately calibrated plastic dropper) of saliva using a marked transfer pipette to a vial containing DRUL buffer or PrimeStore MTM, and return the sample in the mail or via courier. Patients will be provided with a Saliva Specimen Collection Kit. This Kit contains the following items (Table 6). Equivalent supplies* may be used.

TABLE 6
Saliva Specimen Collection Kit contents

| Item | Manufacturer/Supplier | Catalog # |
|---|---|---|
| 2 ml vial filled with 1.2 ml DRUL buffer | DRUL buffer: 1.2 ml, as above | DRUL buffer: NA |
| OR | *Vials: Dynamic Diagnostics | Vials: 374501-BR |
| | (Assembled in RU CGL) | |
| 5 ml vial filled with 1.5 ml PrimeStore | Longhorn Vaccines and | LH-PSMTM-5-50 |
| MTM | Diagnostics, LLC/Thomas | |
| | Scientific | |
| *Transfer pipette | VWR | 414004-018 |
| *Medicine cup | SOLO | 100-2050 |
| *Instruction sheet (may be modified to | Example below, provided by RU | NA |
| accommodate your specific supplies) | CGL | |
| *Biological substance shipping container | Uline | S-18284 |
| w/biohazard bag, Category B (if | | |
| shipping through the mail) | | |

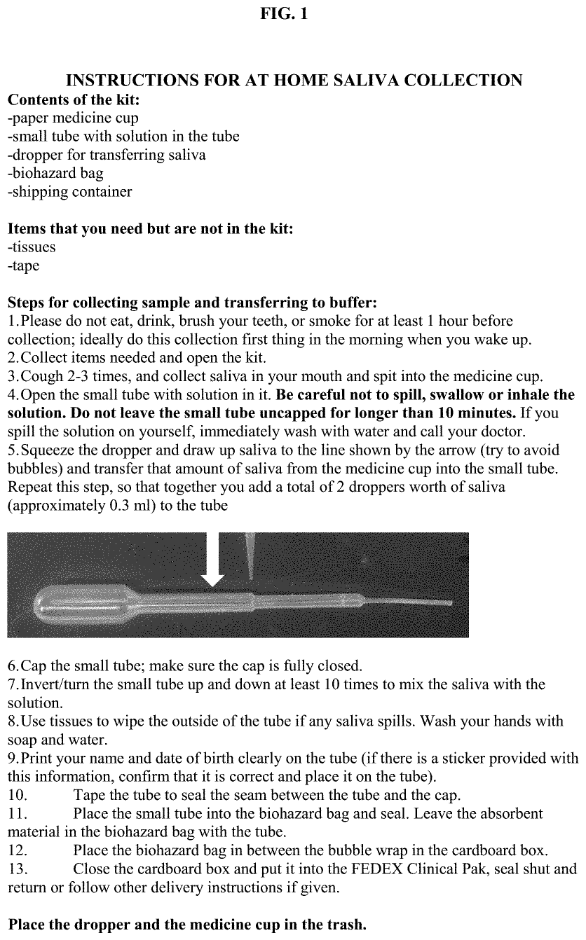

Samples must be transported to the RU CGL. They may be shipped in accordance with the International Air Transport Association (IATA) regulations as an infectious substance, by medical courier, or by other means of transportation.
Specific instructions on packaging are provided in the Instruction Sheet included with the Saliva Sample Collection Kit and packing and shipping material is provided. A example Instruction Sheet providing instructions for at home saliva collection is provided in FIG. 1.
Samples should be transported overnight at room temperature.

Specimen Storage
Specimen may be stored for 2 days at room temperature.
   Extracted RNA may be stored at −70 C for up to one year.

Reagent Preparation

To make 100 ml of DRUL buffer:

TABLE 7
Making DRUL buffer

| Reagent | Amount needed | |
|---|---|---|
| 5M Guanidine Thiocyanate | 59.08 | g |
| 0.5% Sarkosyl | 5 ml of 5% Sarkosyl | |
| 25 mM Sodium Acetate (3M), pH 5.5 | 0.83 | ml |

| | |
|---|---|
| Nuclease-free Water | Bring up to 100 ml |

Extraction Procedure

Perform in pre-PCR area/room.
Perform steps 1-4 inside of a biological safety cabinet of class 2 or higher.

### Option 1: Phenol Method

1. Take 800 ul of the sample+buffer received and place in a 2 ml tube.
2. Add 80 ul of 3M sodium acetate, pH 5.5 and mix by inverting tubes.
3. Add 800 ul of acid-phenol:chloroform and vortex.
4. Add 160 ul of OmiPur chloroform: Iso-Amyl Alcohol and vortex.
5. Spin in microcentrifuge at 12,000 g for 10 minutes at 4° C.
6. Remove the aqueous phase (750 ul) and place into a new tube.
7. Add 2 ul GlycoBlue and mix to dissolve.
8. Add 750 ul ice cold 100% isopropanol.
9. Incubate for 15 min at −80° C.; or overnight at −20° C.
10. Spin in microcentrifuge at 21,000×g for 20 minutes at 4° C.
11. Remove the supernatant carefully without disturbing the pellet.
12. Add 1 ml of cold 75% ethanol.
13. Spin in microcentrifuge at 21,000×g for 5 minutes at 4° C.
14. Remove the supernatant carefully and dry pellet for 10 minutes.
15. Bring up in 35 ul of nuclease-free water.

### Option 2: Column-Based Method

1. Take 800 ul of the saliva+buffer and place in a 2 ml tube.
2. Use the manufacturer's instructions for remainder of the procedure.

Plate Set up for PCR

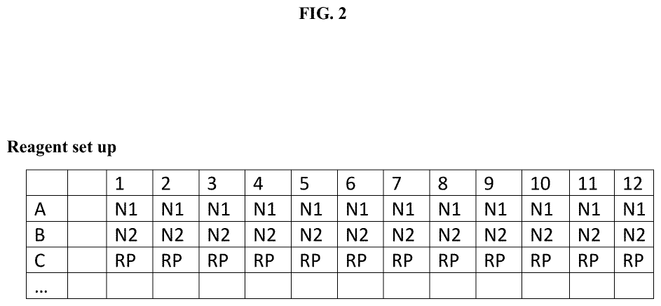

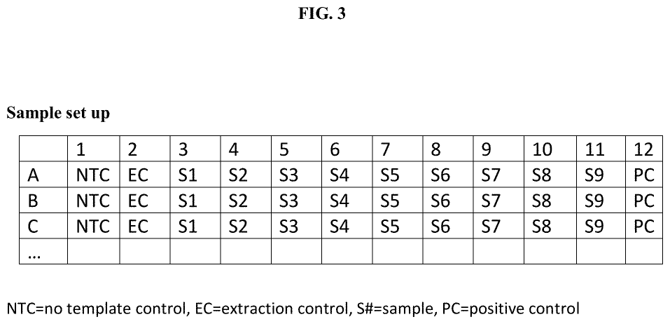

Note: exemplary reagent set up with primers and sample set up grids are provided in FIGS. 2 and 3 respectively.
Perform in a PCR Area/Room, Separate from Pre-PCR Area/Room.
1. Make TaqPath Master Mix with each primer set (N1, N2, RNase P). Volume per primer/probe per reaction:
   1.1. 1.5 ul of each primer/probe mix.
   1.2. 5 ul TaqPath 1 Step RT-PCR.
   1.3. 8.5 ul Nuclease-free water.
2. Add 15 ul TaqPath Master Mix for each of the primer sets (N1, N2, and RNase P across each row) for each patient sample, NTC, positive control, and extraction control. See FIG. 2.
3. Add 5 ul positive control (PC) to 3 wells each containing separate primer sets (N1, N2, and RNase P). See FIG. 3.
4. Add 5 ul extraction control (EC) to 3 wells each containing separate primer sets (N1, N2, and RNase P). See FIG. 3.
5. Add 5 ul nuclease-free water (NTC) to 3 wells each containing separate primer sets (N1, N2, and RNase P). See FIG. 3.
6. Add 5 ul of each sample to 3 wells each containing separate primer sets (N1, N2, and RNase P). See FIG. 3.
7. Seal the plate with Microfilm B.
8. Spin the plate in refrigerated centrifuge at 3000 RPM for 2 minutes at 4 (C.

PCR Run
1. Place the plate into the PCR machine.
2. Use the following parameters (Table 8).

TABLE 8
Protocol “SARS-nCoV-2 test”

| Stage | Temp | Time | | Repeat |
|---|---|---|---|---|
| 1 | 25° C. | 2 | minutes | X1 |
| 2 | 50° C. | 15 | minutes | X1 |
| 3 | 95° C. | 2 | minutes | X1 |
| 4, Step 1 | 95° C. | 3 | seconds | X45 |
| 4, Step 2 | 55° C. | 30 | seconds | |

3. If the protocol is pre-programmed, select the protocol “SARS-nCoV-2 test” from the Saved Files library and tap RUN.
4. Review information on the screen then tap OK.
5. Tap OK in the dialog box to start run.

Data Analysis
1. In the Detected Instruments pane on the Home window of CFX Maestro software, right-click the target instrument and select Retrieve Data Files.

TABLE 9
Expected Results

| | Expected Results | | |
|---|---|---|---|
| Type | N1 primer | N2 primer | RNase P |
| NTC | Neg, >40 Ct | Neg, >40 Ct | Neg, >40 Ct |
| Positive control | Pos, <40 Ct | Pos, <40 Ct | Neg, >40 Ct |
| Extraction control | Neg, >40 Ct | Neg, >40 Ct | Pos, <40 Ct |
| Sample | Pos if <40 Ct | Pos if <40 Ct | Pos, <40 Ct |
| | Neg if >40 Ct | Neg if >40 Ct | |

1. If the controls are expected as above and the sample are both positive for N1 and N2, the test is valid, regardless of whether the sample is positive for RNase P. The sample is positive.
2. If the controls are expected as above and the sample is negative for both N1 and N2, the test is valid and the sample is negative.
3. If the controls are expected as above and the sample is positive for either N1 or N2 but not both, the test is inconclusive. Repeat from extraction and PCR.
4. If the controls are expected as above and the sample is negative for both N1 and N2, but the sample is also negative for RNase P, the test is invalid. Repeat PCR if extracted RNA is available. If not available, re-extract and repeat PCR. If no remaining material is available, request another sample.
5. If the extraction control is positive, the extraction reagents may be contaminated. Repeat with new extraction reagents.
6. If the NTC is positive, the PCR reagents may be contaminated. Repeat with new PCR reagents.
7. Repeat extractions may be performed on 700 ul of input volume.

Quality Control
Quality control of reagents including lot to lot testing will be performed per NYS CLEP standards, including for controls.
Include an extraction control sample with each extraction and add to PCR.
Include a positive control and NTC in each PCR run.

Limitations
This test is a laboratory developed test that has been authorized by NYS under an Emergency Use Authorization (EUA) for use by authorized laboratories (www.fda.gov/emergency-preparedness-and-response/counterterrorism-and-emerging-threats/coronavirus-disease-2019-covid-19). This laboratory developed test is not approved by the FDA.
Performance of this test has only been evaluated in saliva samples placed into DRUL buffer or PrimeStore MTM,
Negative results do not preclude SARS-Cov-2 infection.
   Factors such as the optimal sample type or sample collection timing in the course of disease have not been evaluated. Multiple tests from the same patient may be required. [2]
   Mutations within the primer and/or probe binding regions of the viral genome could affect primer binding and therefore may result in failure to detect the presence of virus,
   Self-collection technique or transport conditions may have affected the quality of the sample received.

Performance Characteristics/Validation

### Limit of Detection (LOD)

The specimen matrix was made using 5 pooled saliva samples and DRUL buffer or PrimeStore MTM spiked with 10-fold serial dilutions of synthetic RNA as specified in the table below and extracted using the Phenol extraction method or Column-based method. The sample was plated in triplicates at each concentration of RNA. The limit of detection (LOD) is 1 copy/ul for both buffers and both extraction methods. The results of the four different combinations are shown in TABLES 10 through 13.

TABLE 10
Determination of LOD with DRUL buffer and phenol extraction.

| | RNA concentration (copies/ul) | | | | | |
|---|---|---|---|---|---|---|
| | 100 | 10 | 1 | 0.1 | 0.01 | 0 |
| Primer N1 Mean Ct | 27.83 | 31.13 | 34.83 | 36.94* | NA | NA |
| Primer N1 Standard | 0.09 | 0.29 | 0.87 | 0.59 | NA | NA |
| Deviation | | | | | | |
| Primer N2 Mean Ct | 29.39 | 32.91 | 36.35* | 40.33 | NA | NA |
| Primer N2 Standard | 0.17 | 0.17 | 0.10 | 0.66 | NA | NA |
| Deviation | | | | | | |

*Mean of two of three replicates (the 3^{rd} well had no value)

TABLE 11
Determination of LOD with PrimeStore
MTM and phenol extraction.

| | RNA concentration (copies/ul) | | | | | |
|---|---|---|---|---|---|---|
| | 100 | 10 | 1 | 0.1 | 0.01 | 0 |
| Primer N1 Mean Ct | 28.00 | 31.28 | 35.12 | 37.40 | NA | NA |
| Primer N1 Standard | 0.29 | 0.18 | 0.32 | 0.13 | NA | NA |
| Deviation | | | | | | |
| Primer N2 Mean Ct | 29.42 | 32.83 | 37.07 | 41.99 | 41.59* | NA |
| Primer N2 Standard | 0.17 | 0.27 | 0.15 | 2.82 | 0.64 | NA |
| Deviation | | | | | | |

*Mean of two of three replicates (the 3^{rd} well had no value)

TABLE 12
Determination of LOD with DRUL buffer
and column-based extraction.

| | RNA concentration (copies/ul) | | | | | |
|---|---|---|---|---|---|---|
| | 100 | 10 | 1 | 0.1 | 0.01 | 0 |
| Primer N1 Cycle time | 27.29 | 30.56 | 33.90 | 38.15 | NA | NA |
| Primer N1 Standard | 0.05 | 0.18 | 0.35 | 0.88 | NA | NA |
| deviation | | | | | | |
| Primer N2 Cycle time | 30.03 | 33.73 | 36.85 | 40.93* | NA | NA |
| Primer N2 Standard | 0.04 | 0.03 | 0.58 | 1.36 | NA | NA |
| deviation | | | | | | |

*Mean of two of three replicates (the 3^{rd} well had no value)

TABLE 13
Determination of LOD with PrimeStore
MTM and column-based extraction.

| | RNA concentration (copies/ul) | | | | | |
|---|---|---|---|---|---|---|
| | 100 | 10 | 1 | 0.1 | 0.01 | 0 |
| Primer N1 Cycle time | 28.59 | 31.99 | 35.05 | NA | NA | NA |
| Primer N1 Standard | 0.14 | 0.24 | 0.04 | NA | NA | NA |
| deviation | | | | | | |
| Primer N2 Cycle time | 31.18 | 35.08 | 38.87 | NA | NA | NA |
| Primer N2 Standard | 0.09 | 0.05 | 0.59 | NA | NA | NA |
| deviation | | | | | | |

Confirmation of Limits of Detection (LOD)

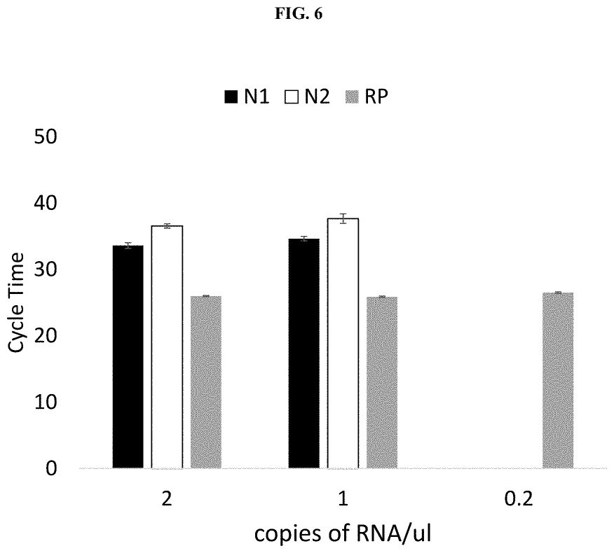

The LOD was confirmed with 5 pooled saliva samples and DRUL buffer or PrimeStore MTM spiked with 2, 1, and 0.2 copies/ul of synthetic RNA, extracted using the Phenol extraction or Column-based method and plated in replicates of 20. We confirmed that our LOD is 1 copy/ul in 20 of 20 replicates in both DRUL buffer and PrimeStore MTM (TABLES 14 through 17 and FIG. 6). This is equivalent to 5 copies of RNA in 5 ul of volume added to the PCR reaction well.

TABLE 14
Confirmation of LOD in DRUL buffer and phenol extraction.

| RNA concentration (copies/ul) | 2 | 1 | 0.2 |
|---|---|---|---|
| Number Positive/Number Tested | 20/20 | 20/20 | 17/20 |
| Primer N1 Cycle time | 33.68 | 34.97 | NA |
| Primer N1 Standard deviation | 0.37 | 0.42 | NA |
| Positive/Tested | 20/20 | 20/20 | 17/20 |
| Primer N2 Cycle time | 35.39 | 36.77 | NA |
| Primer N2 Standard deviation | 0.25 | 0.66 | NA |

TABLE 15
Confirmation of LOD in PrimeStore MTM and phenol extraction.

| RNA concentration (copies/ul) | 2 | 1 | 0.2 |
|---|---|---|---|
| Positive/Tested | 20/20 | 20/20 | 11/20 |
| Primer N1 Cycle time | 34.16 | 35.14 | NA |
| Primer N1 Standard deviation | 0.42 | 0.51 | NA |
| Positive/Tested | 20/20 | 20/20 | 16/20 |
| Primer N2 Cycle time | 36.55 | 37.22 | NA |
| Primer N2 Standard deviation | 0.32 | 0.60 | NA |

TABLE 16
Confirmation of LOD in DRUL buffer
and column-based extraction.

| RNA concentration (copy/ul) | 2 | 1 | 0.2 |
|---|---|---|---|
| Positive/Tested | 20/20 | 20/20 | 17/20 |
| Primer N1 Cycle time | 33.61 | 34.65 | NA |
| Primer N1 Standard deviation | 0.43 | 0.34 | NA |
| Primer N2 Cycle time | 36.57 | 37.67 | NA |
| Primer N2 Standard deviation | 0.32 | 0.73 | NA |

TABLE 17
Confirmation of LOD in PrimeStore
MTM and column-based extraction.

| RNA concentration (copy/ul) | 2 | 1 | 0.2 |
|---|---|---|---|
| Positive/Tested | 20/20 | 20/20 | 13/20 |
| Primer N1 Cycle time | 33.96 | 34.91 | NA |
| Primer N1 Standard deviation | 0.38 | 0.47 | NA |
| Primer N2 Cycle time | 36.92 | 37.74 | NA |
| Primer N2 Standard deviation | 0.40 | 0.71 | NA |

Clinical Evaluation

A clinical evaluation study was conducted as follows. Synthetic RNA was spiked into five different individual specimen matrices at 2×, 4×, 6×, 8×, 10×, and 100× the LOD (1 copy/ul) and extracted using the Phenol extraction method or Column-based method. In addition, 10 negative specimen matrices were assayed. Mean cycle times and standard deviations are shown in TABLE 18 through 21.

TABLE 18
Clinical Evaluation with DRUL buffer
and phenol extraction method.

| | RNA concentration (copies/ul) | | | | | | |
|---|---|---|---|---|---|---|---|
| | 100X | 10X | 8X | 6X | 4X | 2X | 0 |
| Positive/Tested | 5/5 | 5/5 | 5/5 | 5/5 | 5/5 | 5/5 | 0/10 |
| Primer N1 Cycle | 27.37 | 30.61 | 31.04 | 31.35 | 31.63 | 32.68 | NA |
| time | | | | | | | |
| Primer N1 Standard | 0.43 | 0.38 | 0.31 | 0.42 | 0.24 | 0.37 | NA |
| deviation | | | | | | | |
| Primer N2 Cycle | 28.85 | 32.50 | 32.62 | 33.06 | 33.89 | 34.92 | NA |
| time | | | | | | | |
| Primer N2 Standard | 0.53 | 0.66 | 0.40 | 0.48 | 0.40 | 0.84 | NA |
| deviation | | | | | | | |

TABLE 19
Clinical Evaluation with PrimeStore
MTM and phenol extraction method.

| | RNA concentration (copies/ul) | | | | | | |
|---|---|---|---|---|---|---|---|
| | 100X | 10X | 8X | 6X | 4X | 2X | 0 |
| Positive/Tested | 5/5 | 5/5 5 | 5/5 | 5/5 | 5/5 | 5/5 | 0/10 |
| Primer N1 Mean Ct | 27.33 | 30.72 | 30.90 | 31.39 | 31.78 | 32.89 | NA |
| Primer N1 Standard | 0.41 | 0.28 | 0.40 | 0.56 | 0.41 | 0.40 | NA |
| Deviation | | | | | | | |
| Primer N2 Mean Ct | 28.50 | 32.18 | 32.32 | 32.81 | 33.42 | 34.42 | NA |
| Primer N2 Standard | 0.44 | 0.48 | 0.49 | 0.61 | 0.35 | 0.39 | NA |
| Deviation | | | | | | | |

TABLE 20
Clinical Evaluation in DRUL buffer
and column-based extraction method.

| | RNA concentration (copies/ul) | | | | | | |
|---|---|---|---|---|---|---|---|
| | 100X | 10X | 8X | 6X | 4X | 2X | 0 |
| Positive/Tested | 5/5 | 5/5 | 5/5 | 5/5 | 5/5 | 5/5 | 0/10 |
| Primer N1 Cycle | 27.28 | 30.69 | 31.34 | 31.48 | 32.11 | 33.16 | NA |
| time | | | | | | | |
| Primer N1 Standard | 0.48 | 0.37 | 0.49 | 0.26 | 0.48 | 0.54 | NA |
| deviation | | | | | | | |
| Primer N2 Cycle | 30.06 | 34.08 | 34.29 | 34.55 | 35.20 | 36.54 | NA |
| time | | | | | | | |
| Primer N2 Standard | 0.83 | 1.05 | 1.32 | 0.85 | 0.92 | 1.06 | NA |
| deviation | | | | | | | |

TABLE 21
Clinical evaluation in PrimeStore MTM buffer
and column-based extraction method.

| | RNA concentration (copies/ul) | | | | | | |
|---|---|---|---|---|---|---|---|
| | 100X | 10X | 8X | 6X | 4X | 2X | 0 |
| Positive/Tested | 5/5 | 5/5 | 5/5 | 5/5 | 5/5 | 5/5 | 0/10 |
| Primer N1 Cycle | 27.68 | 31.40 | 31.88 | 31.94 | 32.71 | 33.80 | NA |
| time | | | | | | | |
| Primer N1 Standard | 0.81 | 0.93 | 1.46 | 1.19 | 1.28 | 1.00 | NA |
| deviation | | | | | | | |
| Primer N2 Cycle | 30.80 | 34.78 | 35.59 | 35.77 | 36.40 | 37.32 | NA |
| time | | | | | | | |
| Primer N2 Standard | 1.18 | 1.08 | 1.78 | 1.67 | 1.74 | 1.66 | NA |
| deviation | | | | | | | |

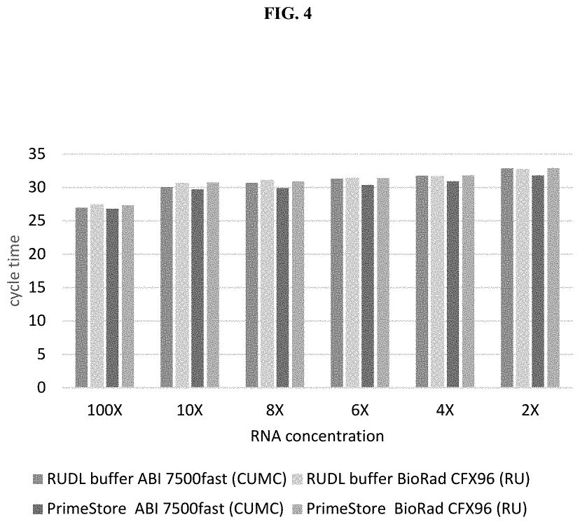

In a second clinical evaluation study, the RNA extracted above using the phenol extraction method from saliva samples in both DRUL buffer and PrimeStore MTM spiked with synthetic RNA at the specific concentrations were also run on a clinically approved EUA assay on the ABI 7500 fast platform, and found similar results (FIG. 4, Tables 22 and 23).

TABLE 22
DRUL buffer assayed on ABI 7500fast platform.

| | RNA concentration (copies/ul) | | | | | | |
|---|---|---|---|---|---|---|---|
| | 100X | 10X | 8X | 6X | 4X | 2X | 0 |
| Positive/Tested | 2/2 | 2/2 | 2/2 | 2/2 | 2/2 | 2/2 | 2/2 |
| Primer N1 Cycle | 26.96 | 30.05 | 30.68 | 31.30 | 31.75 | 32.86 | NA |
| time | | | | | | | |
| Primer N1 Standard | 0.56 | 0.33 | 0.34 | 0.40 | 0.53 | 0.10 | NA |
| deviation | | | | | | | |
| Primer N2 Cycle | 29.60 | 33.70 | 34.95 | 35.36 | 36.96 | 37.79 | NA |
| time | | | | | | | |
| Primer N2 Standard | 0.72 | 1.79 | 1.14 | 0.84 | 0.02 | 0.42 | NA |
| deviation | | | | | | | |

TABLE 23
PrimeStore MTM assayed on ABI 7500fast platform.

| | RNA concentration (copies/ul) | | | | | | |
|---|---|---|---|---|---|---|---|
| | 100X | 10X | 8X | 6X | 4X | 2X | 0 |
| Positive/Tested | 2/2 | 2/2 | 2/2 | 2/2 | 2/2 | 2/2 | 2/2 |
| Primer N1 Cycle | 26.80 | 29.70 | 29.88 | 30.36 | 30.93 | 31.79 | NA |
| time | | | | | | | |
| Primer N1 Standard | 0.26 | 0.43 | 0.42 | 0.66 | 0.56 | 0.37 | NA |
| deviation | | | | | | | |
| Primer N2 Cycle | 28.91 | 32.01 | 31.76 | 32.96 | 34.04 | 37.48 | NA |
| time | | | | | | | |
| Primer N2 Standard | 0.31 | 0.04 | 0.27 | 0.71 | 0.08 | 2.82 | NA |
| deviation | | | | | | | |

In a third clinical evaluation study, we obtained 30 nasopharyngeal swab samples from patients who tested positive at a New York City Department Health Public Health Laboratory. The samples had crossing thresholds (ct) ranging from 17.3 to 37.6 using the FDA approved Cephied Xpert Xpress SARS-CoV-2 assay. 300 ul aliquots of each these NP swab samples were placed in DRUL buffer, extracted using the phenol method and assayed using the procedure described above. There was 100% concordance between these results with 30 of 30 testing positive by both assays and the cycle times crossing thresholds were highly correlated (r²=0.9553, FIG. 5).

Summary Statement

The Rockefeller Clinical Genomics SARS CoV-2 real-time reverse transcriptase polymerase chain reaction (RT-PCR) test for the qualitative detection of RNA from the SARS-CoV-2 in patient saliva is validated as per as per the Wadsworth Validation Procedure for SARS-CoV-2 LDT. The assay was validated for accuracy of identification of SARS-CoV-2 using the CDC EUA primers. The assay is accurate and precise. The LOD is 1 copy/ul. This assay is ready to be used for qualitative detection of SARS-CoV-2 in patient saliva in the Rockefeller University Clinical Genomics Laboratory (RU CGL).

### REFERENCES

[1] Chomczynski P & Sacchi N. (1987). “Single-step method of RNA isolation by acid guanidinium thiocyanate-phenol-chloroform extraction”. Anal. Biochem. 162: 156-159. doi:10.1016/0003-2697(87)90021-2. PMID 2440339.
[2] To K K, Tsang O T, Chik-Yan Yip C, Chan K H, Wu T C, Chan J M C, Leung W S, Chik T S, Choi C Y, Kandamby D H, Lung D C, Tam A R, Poon R W, Fung A Y, Hung I F, Cheng V C, Chan J F, Yuen K Y. Consistent detection of 2019 novel coronavirus in saliva. Clin Infect Dis. 2020 Feb. 12. pii: ciaa149. doi: 10.1093/cid/ciaa149.

### Example 3

### Sars-Cov-2 Saliva Testing with Drul at Scale

One of the limits and opportunities for scaling molecular diagnostic testing relates to pooling of clinical samples. It is currently feasible to test large numbers within a single laboratory, which may have capacity to test 1,000-10,000 or more clinical samples in a day. However, the demand for such testing, particularly in the context of a global pandemic such as the current COVID-19 crisis, is disproportionately greater than what labs can now manage.

Such clinical testing, typically for the presence of nucleic acids from an infectious agent, can be accomplished precisely by modern tests, the most sensitive of which is polymerase chain reaction (PCR) testing. PCR testing for COVID-19 is currently the gold standard, as it is both sensitive, specific, and can detect virus prior to symptom onset (or in asymptomatic individuals), a critical need in COVID-19.

COVID-19 PCR testing as approved by the FDA, or by NY State CLIA-CLEP laboratories under FDA Emergency Use Authorization, typically tests for multiple unique fragments of SARS-CoV-2 RNA from the N gene (N1 and N2), and tests for a positive control that should be present in all individuals (typically the Ribonuclease P gene, RP). It is possible to detect all three products simultaneously in a single PCR reaction, using three probes that fluoresce at different wavelengths, using appropriate multi-laser equipped PCR machines, which can detect 96 samples (e.g. the ABI 7500 Fast PCR machine or equivalent) or 384 (CFX Opus 384 Real-Time PCR Systems) samples simultaneously.

Additional limits on scaling testing involve technical and logistical issues. To be able to screen at large scale—for example all children (preK-grade 12, 1.2 million), teachers (-86,000, including paraprofessionals) and parents (˜1.8 million) twice a week—would include the ability to collect safely and test˜500,000 clinical samples per day. The initial CDC standards for COVID-19 testing involved healthcare providers wearing personal protective equipment administering uncomfortable nasopharyngeal swabs (NPS, in which the upper nasal turbinates are scrapped with a plastic swab), a procedure not well tolerated by children (or adults), then placing those swabs in viral transport media (a solution designed to grow live virus), and transporting those securely to a testing facility equipped to handle potential infectious material. Moreover, the obligation to use this equipment and material placed severe strains on supply chains, and correspondingly limited testing when it was most needed during the first peak of the CoVID-19 pandemic.

To address these challenges, our laboratory developed low cost and safe saliva testing as an alternative approach to testing for SARS-CoV-2, applying for and receiving approval from NY State under Early Use Authorization (EUA) from the Wadsworth Center NY State Department of Health (NYSDOH), following FDA requirements on Apr. 9, 2020. This test is detailed in Example 2 above. The rationale for testing saliva relates to studies of SARS-CoV done in 2011, demonstrating that salivary gland duct cells express the ACE2 receptor (also the target of SARS-CoV-2), and that these cells are highly infected by SARS-CoV in primate models [1], and saliva testing by multiplex PCR was found to improve detection of respiratory viruses relative to NPS specimens [2]. In early clinical studies of COVID-19 patients undertaken in Hong Kong, SARS-CoV-2 was detected by RT-PCR in 11/12 patients [3], and saliva testing was effectively instituted by the Hong Kong Center for Health Protection, Communicable Disease in March 2020.

It has subsequently become increasingly evident that the use of saliva is a general solution for COVID-19 testing [4][5], and on Aug. 16, 2020 the FDA issued an emergency use authorization (EUA) to Yale School of Public Health for its SalivaDirect COVID-19 diagnostic test, which used a new method of processing saliva samples when testing for COVID-19 infection. The sensitivity of SARS-CoV-2 detection from saliva has been reported to be comparable to nasopharyngeal swabs in mildly symptomatic and asymptomatic patients with Covid-19 [6], although results likely depend on the test developed, its accompanying sensitivity and specificity, and clinical status of individuals [7]. Despite its promise, challenges with saliva testing have been difficult to overcome, as different samples have various degrees of viscosity, making a standardized “one-fits-all” protocol difficult to manage, and in general many of the now prevalent saliva assays have relatively poor limits of detection. For example, the Yale SalivaDirect assay has a limit of detection of 6-12 copies/ul saliva. Moreover, methods for scalable RNA preparation from saliva samples, such as magnetic bead purification of RNA, are extremely sensitive to both viscosity and potential inhibitory agents in saliva.

Efforts to overcome these limitations have included attempts to study RNA from saliva by qRT-PCR that skips RNA purification steps. Such approaches are relatively fast, but have in general suffered from loss of sensitivity, as purified RNA is the highest quality reagent for qRT-PCR testing. Nonetheless, saliva-based assays have been reported to be more reliable than NPS testing in some instances (Wyllie A L et al (2020) N Engl J Med doi:10.1056/NEJMC202016359).

We present here a new, safe, inexpensive and scalable means of saliva testing. This approach has been validated per the Wadsworth Validation Procedure for SARS-CoV-2 as a lab developed test, and submitted to the Wadsworth Center NYSDOH. We have developed a set of safe yet powerful lysis buffers (Darnell Rockefeller University Lab lysis buffers 1-2, DRUL lysis buffers) that can, in small volumes (300 ul), be combined with similarly small volumes of self-collected saliva from individuals in their home, workplace, or elsewhere. We have shown that DRUL lysis buffer is safe for shipping and lab personnel handling, as it effectively neutralizes coronavirus on contact and stabilizes viral RNA for days at room temperature, allowing subsequent transfer to the testing laboratory. DRUL lysis buffer uses standard molecular biology reagents, addressing the issue of limited supply limitations and costs—the net cost of reagents used for analysis of one sample from saliva acquisition through RT-PCR analysis is ˜$2.50/sample. Additional procedures allow the testing kits to be further sterilized with a brief heat treatment upon receipt in the lab, which further prepares the RNA present in saliva for scaled testing, and allows sample preparations to be done on laboratory benches (rather than BSL2 hoods), facilitating scaling. A robotic testing unit prepares purified RNA within 25 minutes for multiplex PCR testing for SARS-CoV-2. The net result is a test that is extremely sensitive (0.5 viral copies/ul saliva), with a limit of detection 12-24 fold greater than that of related assays such as the Yale SalivaDirect assay (6-12 viral copies/ul).

DRUL2 Saliva Testing Protocol: Results and Methods

Validation: Molecular Assay SARS-CoV-2 Saliva Laboratory Developed Test

Test Name—Rockefeller Clinical Genomics Laboratory SARS CoV-2 assay 2 (RUCGL SCV)

Test Purpose—We have developed a SARS-CoV-2 molecular diagnostic test to be performed for the in vitro qualitative detection of RNA from the SARS-CoV-2 virus in saliva samples from patients. We developed this with high clinical standards, as an Emergency Use Authorization (EUA) request, for use as recommended for testing by public health authority guidelines or for screening of asymptomatic individuals and diagnosis of symptomatic individuals. This laboratory developed test was developed to be performed in our New York state-permitted, CLIA certified high-complexity laboratory.

Test Summary and Explanation—The DRUL2 Rockefeller University Clinical Genomics Laboratory SARS CoV-2 assay 2 (RUCGL SCV2, also known as the DRUL2 assay) is a real-time PCR assay using primers and probe sets to detect RNA from SARS CoV-2. The test uses saliva samples self-collected at home, in a healthcare provider's office, or elsewhere and delivered or shipped to the RUCGL. Molecular analysis uses multiplex primer-probes used in the FDA-approved EUA protocol, Triplex CII-SARS-CoV-2 rRT-PCR test (Columbia University Laboratory of Personalized Genomic Medicine) [1] with real-time PCR methodology. The multiplex primers target two separate regions of the viral nucleocapsid (N) gene, N1 and N2. Also included is an internal control targeting the human RNase P (RP) gene. All three targets are detected in a single assay in multiplex, each with a unique fluorophore-quencher combination.

Intended Use—RUCGL SCV2 is a real-time reverse transcriptase polymerase chain reaction (RT-PCR) test for the qualitative detection of RNA from the SARS-CoV-2 in patient saliva. The test is intended to be used for detection of viral RNA in saliva specimens collected in a transport medium that potentially inactivates the virus while stabilizing the viral RNA. Saliva specimens can be either self-collected or collected at a healthcare provider's office and returned to the Rockefeller University Clinical Genomics Laboratory (RUCGL) for testing. The test uses a stringent extraction protocol that provides for a highly sensitive detection of SARS CoV-2 using primers and probes from the Triplex CII-SARS-CoV-2 rRT-PCR TEST (Columbia University Laboratory of Personalized Genomic Medicine) EUA [1] with real-time PCR methodology. The assay was validated as per the Wadsworth Validation Procedure for SARS-CoV-2 LDT.

Principles of the Procedure—Saliva samples are collected at home, in a healthcare provider's office or elsewhere and added to buffer at the site of collection, stabilizing RNA and inactivating virus at the point of collection. The extraction reagents and method used here is a modified version of the original Rockefeller Clinical Genomics Laboratory SARS CoV-2 assay (described in Example 2), which itself is a modification of the guanidinium thiocyanate-phenol-chloroform extraction method [2] used in molecular biology for denaturing protein from a variety of sources and subsequently isolating RNA. These reagents are widely available and economic, and variations of the method that include guanidinium thiocyanate are used in many commercial kits. After samples are transported to the RUCGL, RNA is purified using an automated version of standard molecular biology magnetic-bead based extraction method, and then reverse transcribed into cDNA. cDNA is amplified using multiplexed primers and probes described in an FDA approved EUA (Triplex CII-SARS-CoV-2 rRT-PCR Test; Columbia University Laboratory of Personalized Genomic Medicine) [1]. In the PCR assay, the probes are hydrolyzed during primer amplification of the primer specific region, cleaving and releasing fluorophores from the quencher, resulting in fluorescence signals proportional to the target sequences present in the sample. The fluorescence intensities are measured using an ABI 7500 Fast Dx Real-Time PCR Detection System.

DRUL2 Saliva Protocol: Methods and Results

Reagants

Sample Collection Kits (described below in Sample Collection) are either delivered to individuals to be tested or given to them by a healthcare providers. A component of the kit is a vial containing the Darnell Rockefeller University Lab (DRUL2) buffer. The DRUL2 buffer contains guanidine thiocyanate, sarkosyl, and sodium acetate (Table 24). This buffer will denature proteins, stabilize RNA and also inactivate virus. Patients are instructed to add saliva to this buffer.

### DRUL2 Buffer Reacents:

TABLE 24
DRUL2 buffer

| Reagent | Purpose | Manufacturer | Catalog | Storage |
|---|---|---|---|---|
| Guanidine | Protein denaturing agent, | Fisher | BP221-1 | Room |
| Thiocyanate | Isolation of RNA | BioReagents | | temp |
| Sarkosyl | Cell lysis, detergent | Fisher | BP234-500 | Room |
| | | BioReagents | | temp |
| Sodium Acetate (3M), | Precipitation of RNA | Invitrogen | AM9740 | Room |
| pH 5.5, RNase free | | | | temp |
| Nuclease-free | Dilution | Ambion | AM9932 | Room |
| Water | | | | temp |

Samples received in DRUL2 buffer are extracted using a magnetic bead-based method. Extracted RNA is then used in a PCR reaction.

RNA Extraction Reagents:

TABLE 25
Extraction Reagents.

| Reagent | Manufacturer | Catalog # | Storage |
|---|---|---|---|
| Dithiothreitol | Thermo Fisher Scientific | R0861 | −20° C. |
| Binding buffer | See below | N/A | Room temp |
| CleanNGS | Bulldog Bio | CNGS-0500 | 2-8° C. |
| Ethanol | Decon Labs/ThermoFisher | V1016 | Room temp |
| Nuclease-free | Thermo Fisher Scientific | AM9932 | Room temp |
| water | | | |

Binding Buffer Reagents:

TABLE 26
Binding Buffer.

| Reagent | Manufacturer | Catalog # | Storage |
|---|---|---|---|
| PEG 8000 | Promega | V3011 | Room temp |
| 5M NaCl | Invitrogen | AM9759 | Room temp |
| 0.1M Trisodium | Sigma | 51804 | Room temp |
| Citrate | | | |
| Tween 20 | Sigma | P1379 | Room temp |
| 0.1N HCl | Fisher Scientific | A508-P500 | Room temp |
| Nuclease-free Water | Thermo Fisher | AM9932 | Room temp |
| | Scientific | | |
| Proteinase K** | Creative BioMart | CMC05 | Room temp |
| (optional) | | | |

**The isolation method works with or without proteinase K. If proteinase K is utilized, it may be used in preferred conditions of Proteinase K at 55 ug/ml final concentration in mix of saliva + DRUL + binding buffer.

PCR Reagents:

TABLE 27
PCR Reagents.

| Reagent | Manufacturer | Catalog # | Storage |
|---|---|---|---|
| TaqPath 1 Step RT-PCR | Life Tech | A15300 | −20° C. |
| Multiplex primer/ | LGC Biosearch | Custom (see | −20° C., |
| probe set | Technologies | below) | dark |
| Nuclease-free | Thermo Fisher | AM9932 | Room temp |
| water | Scientific | | |

Primer/Probes:

TABLE 28
Primer/Probes.

| | 5′ | | 3′ |
|---|---|---|---|
| Custom Primer/Probes*** | Modification | Sequence | Modification |
| SARS-COV-2 forward primer for N1 | — | GACCAGGAACTAATCAGACAAGG | — |
| target (nCoV_NF1) | | (SEQ ID NO: 13) | |
| SARS-COV-2 reverse primer for N1 | — | TCAACCACGTTCCCGAAGG | — |
| target (nCoV_NR1) | | (SEQ ID NO: 14) | |
| SARS-Cov-2 FAM Probe for N1 target | FAM | CGACATTCCGAAGAACGCTGAAGCG | BHQ-1 |
| (nCoV_NP1) | | (SEQ ID NO: 15) | |
| SARS-COV-2 forward primer for N2 | — | GCCATCAAATTGGATGACAAAGATC | — |
| target (nCoV_NF2) | | (SEQ ID NO: 16) | |
| SARS-COV-2 reverse primer for N2 | — | TAGGCTCTGTTGGTGGGAATG | — |
| target (nCoV_NR2) | | (SEQ ID NO: 17) | |
| SARS-COV-2 Quasar 670 Probe for N2 | Quasar 670 | CATTTTGCTGAATAAGCATATTGACGC | BHQ-3 plus |
| target (nCoV_NP2) | | (SEQ ID NO: 18) | |
| RNAse P forward primer (RP_F) | — | AGATTTGGACCTGCGAGCG | — |
| | | (SEQ ID NO: 19) | |
| RNAse P reverse primer (RP_R) | — | GAGCGGCTGTCTCCACAAGT | — |
| | | (SEQ ID NO: 20) | |
| RNAse P CAL Fluor Orange 560 probe | CAL Fluor | TTCTGACCTGAAGGCTCTGCGCG | BHQ-1 |
| (RP_P) | Orange 560 | (SEQ ID NO: 21) | |

***The Probes are all manufactured by LGC Bioresearch Tecnologies. All probes are stored at Room Temperature

Controls:

TABLE 29
Controls.

| Reagent | Manufacturer | Catalog # | Storage |
|---|---|---|---|
| Positive control | BEI Resources | NR-52358 | −20 C. |
| Quantitative synthetic RNA | | | |
| from SARS-Related Coronavirus 2 | | | |
| Negative control | Thermo Fisher | AM9932 | Room |
| nuclease-free water | Scientific | | temp |
| Extraction control | Darnell Lab | NA | −80 C. |
| HeLa cells (1.25 × 105 | | | |
| cells in 0.8 ml) | | | |

Equipment and Materials Used
ABI 7500Fast Dx PCR Detection System
Thermofisher KingFisher Apex
KingFisher 96 deep-well plate, polypropylene, Cat #95040450
Pipettes P10, P20, P200
Aerosol barrier pipette tips (DNase/RNase free)
ThermoFisher 0.1 ml PCR reaction plates (96 well plates), Cat #4366932
BioRad Microseal B PCR plate seals, Cat #MSB1001
0.2 ml PCR reaction plates (96 well plates)
Microseal B PCR plate seals
Disposal gloves, gowns, masks

Warnings and Precautions
All samples should be handled with universal precautions.
Use personal protective equipment when handling samples and reagents.
Take care not to introduce contamination with amplicons from previous reactions.
   Use separate spaces for extraction and amplification.
   Use dedicated equipment and personnel protective equipment in those rooms or areas.
   Always use barrier pipette tips.
   Change gloves between specimens.
   Keep reagents capped or covered whenever possible.
   Clean up spills immediately.
   Use RNase Away to decontaminate pipettes, work surfaces, and centrifuges to minimize risk of contamination.
   Dispose of chemical or biological waste according to institutional procedures.

Reagent Storage, Handling and Stability
Use storage temperatures listed above.
For reagents without manufacturer specified expiration dates, the RUCGL uses the date one year after receipt as the expiration date.
Positive control RNA should be aliquoted to avoid freeze thaw cycles (see Reagent Preparation).

Specimen Collection, Handling and Storage

Saliva collected and transported as specified below.

Specimen Collection Procedure

Patients will collect saliva at home or other locations such as a health care provider's office. Patients will be provided with a Saliva Specimen Collection Kit (Table 30). Patients will add approximately 300 ul of saliva using a transfer pipette to a vial containing DRUL2 buffer, and return the sample in a local secure dropbox, in the mail or via courier. Appropriate shipping materials may be provided.

TABLE 30
Saliva Specimen Collection Kit contents

| Item | Manufacturer/Supplier | Catalog # |
|---|---|---|
| 1 ml vial filled with 0.3 | *Vials: Brooks | Vials: 374501-BR |
| ml DRUL2 buffer | DRUL2 buffer: 0.3 ml, | DRUL2 buffer: NA |
| | as above (Assembled in | |
| | RUCGL) | |
| *Transfer pipette | VWR | 414004-018 or |
| | | equivalent |
| *Medicine cup | SOLO | 100-205 |
| *Instruction sheet (may be modified | Example below, provided | NA |
| to accommodate specific supplies) | by RUCGL | |
| *Biological substance shipping | Uline | S-18284 |
| container w/biohazard bag, Category | | |
| B (if shipping through the mail) | | |

*Equivalent supplies may be used

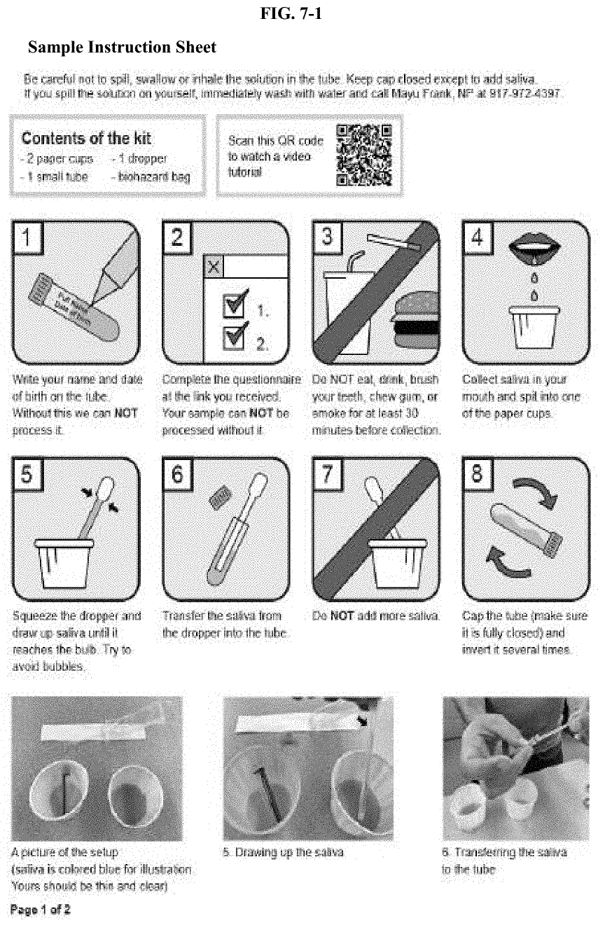

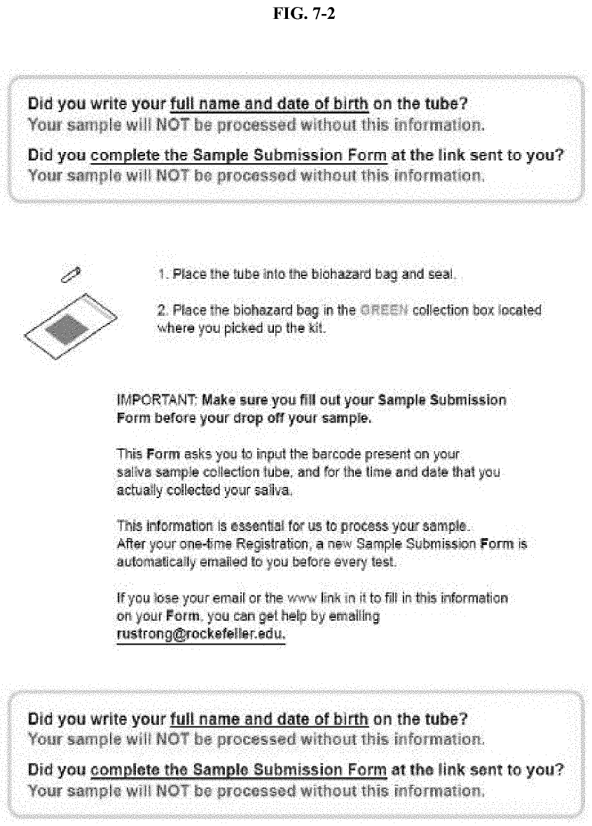

A Sample Instructions Sheet providing instructions for Saliva Collection for SARS-COV2-Testing is provided in FIG. 7.

Specimen Delivery Procedure
Samples must be transported to the RUCGL. They may be dropped off directly in a designated space, or shipped in accordance with the International Air Transport Association (IATA) regulations as an infectious substance, by medical courier, or by other means of transportation.
Specific instructions on packaging are provided in the Instruction Sheet included with the

Saliva Sample Collection Kit (an example is provided in FIG. 6) and packing and shipping material may be provided.

Samples should be shipped overnight at room temperature.

Specimen Storage
Specimen may be stored for 2-5 days at room temperature.
Extracted RNA may be stored at −70° C. for up to one year.

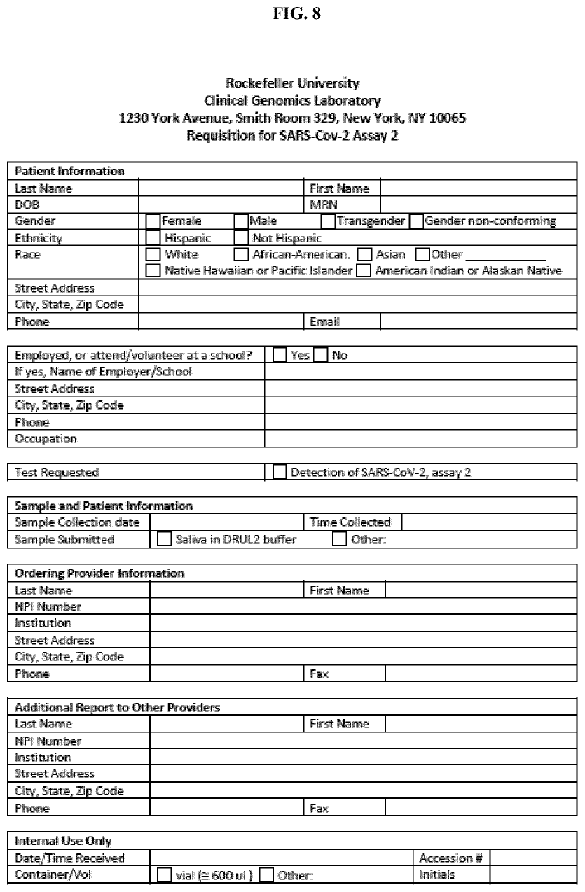

An exemplary Requisition request for the SARS-Cov-2 Assay-2 test is provided in FIG. 8.

Reagent Preparation

To make 100 ml of DRUL2 buffer:

TABLE 31
Making DRUL2 buffer.

| Reagent | Amount needed | | Final Concentration |
|---|---|---|---|
| Guanidine Thiocyanate | 59.08 | g | 5M |
| 10% Sarkosyl | 5 | ml | 0.5% |
| 3M Sodium Acetate, pH 5.5 | 5.81 | ml | 175 mM |

| | |
|---|---|
| Nuclease-free Water | Bring up to 100 ml |

To make 100 ml Binding Buffer:

TABLE 32

| Reagent | Amount needed | | Final Concentration | |
|---|---|---|---|---|
| PEG 8000 | 20 | g | 20% | (w/v) |
| 5M NaCl | 50 | ml | 2.5M | |
| 0.1M Trisodium Citrate | 10 | ml | 1 | mM |
| Tween 20 | 0.050 | ml | 0.05% | (v/v) |
| 0.1N HCl | 0.056 | ml | 0.56 | mM |
| Nuclease-free Water | Bring up to 100 ml | | | |
| Optional: Proteinase K, | 0.5 | ml | 121 | μg/ml |

20 mg/ml (in 20 mM Tris-HCl
pH 8, 1mM CaCl₂₎

To make wash buffer:

TABLE 33
Wash buffer.

| Reagent | Amount needed |
|---|---|
| Ethanol | 80 ml |
| Nuclease-free Water | Bring up to 100 ml |

To make DTT stock solution:

TABLE 34
DTT Stock solution.

| Reagent | Amount needed |
|---|---|
| DTT powder | 25 g |
| Nuclease-free Water | Bring up to 55.5 ml |

To make 100× positive control stock:

TABLE 35
Positive control stock.

| Reagent | Amount needed |
|---|---|
| Quantitative synthetic RNA from SARS- | 1 ul |
| Related Coronavirus 2 | |
| Nuclease-free Water | 289 ul |

Make 2 ul aliquots and store at −80 C. On day of assay, thaw 1 vial of 100× stock and add 1 ul to 99 ul of nuclease-free water to use in PCR.

To make 100 uM primer/probe stock:

TABLE 36
Primer/probes.
Custom Primer/Probes

SARS-CoV-2 forward primer for N1 target (nCoV_NF1)
SARS-CoV-2 reverse primer for N1 target (nCoV_NR1)
SARS-Cov-2 FAM Probe for N1 target (nCoV_NP1)
SARS-CoV-2 forward primer for N2 target (nCoV_NF2)
SARS-CoV-2 reverse primer for N2 target (nCoV_NR2)
SARS-CoV-2 Quasar 670 Probe for N2 target (nCoV_NP2)
RNAse P forward primer (RP_F)
RNAse P reverse primer (RP_R)
RNAse P CAL Fluor Orange 560 probe (RP_P)

Add nuclease-free water to each tube:

Volume nuclease-free water in ul=“total nmol amount”×10

Store at −80 C, protected from light.

KingFisher Bead Extraction Procedure
1. Heat all sample tubes in kits at 65° C. for 15 minutes in dry oven.
2. Place tubes in 96 well rack and spin at 2000×g for 2 minute at room temperature.
3. Using a multichannel pipettor, add 16 ul of 3M DTT stock to each 600 ul sample (saliva+DRUL2). Mix by pipetting up/down, without introducing bubbles.
4. Remove 200 ul from each sample tube and add to a well in the 96 well deep well plate.
5. Swirl the Binding Buffer to mix the viscous solution then add 165 ul to each sample well. Mix gently so as not to introduce bubbles.
6. Thoroughly mix CleanNGS bead stock solution to disperse beads by swirling and inverting, then add 110 ul CleanNGS beads to each sample well.
7. Prepare 4-96 well deep well plates:
   a. Tip combs
   b. Wash 1: add 0.5 ml 80% ethanol to each well
   c. Wash 2: add 1 ml 80% ethanol to each well
   d. Elution: add 50 ul water to each well
8. Place the 96 well deep well plates on the KingFisher Apex as prompted.
9. Use the RBD protocol (pre-programmed in KingFisher Apex):
   a. Binding
      i. Heat: 65° C.
      ii. Mix: Fast for 5 minutes
      iii. Collect beads: 5 count*
   b. Wash 1
      i. Release beads: Fast, 20 seconds
      ii. Mix: Bottom mix, 10 seconds, looping 2 times; medium mix, 10 seconds
      iii. Collect beads: 3 count, 1 second
   c. Dry
      i. 2 minutes, outside well
   d. Elution
      i. Release beads
      ii. Mix: Bottom mix, 15 seconds, looping 6 times; medium mix, 45 seconds
      iii. Collect beads: 8 count*, 1 second
   e. Final bead
      i. Collect beads: 10 count*, 2 seconds
   f. Hold temperature at 4° C.
10. Use 10 ul of sample for PCR (see below).
   a. Step 10P: Note, if RNA will be used immediately for pooling, see pooling protocol below (Example 10)
11. Freeze remaining RNA eluate at −80° C.
12. Note that to save plastic, Deep-well plates can be reused by soaking in 1% sodium hypochlorite for 10 min at room temperature, rinsed thoroughly with distilled water for 10 minutes and dried.
   a. This has been tested/optimized by spiking samples with known amounts of chemically inactivated SARS-CoV-2 RNA, cleaning plates as above, and re-extracting plates with pre-determined “negative” saliva plus binding buffer and magnetic beads per this protocol. No detectable SARS-CoV-2 (N1, N2) could be detected, while RP values were unchanged.
count refers to number of times the magnetic probes dip into the sample to collect beads

PCR Plate Set Up
1. Make TaqPath Master Mix with multiplex primer set.

Volume per primer/probe per reaction:

| | | |
|---|---|---|
| Nuclease free water | 0.15 | ul |
| nCoV_NF1 (10 uM) | 0.75 | ul |
| nCoV_NR1 (10 uM) | 0.75 | ul |
| nCoV_NP1 (10 uM) | 0.6 | ul |
| nCoV_NF2 (10 uM) | 0.75 | ul |
| nCoV_NR2 (10 uM) | 0.75 | ul |
| nCoV_NP2 (10 uM) | 0.6 | ul |
| RP_F (10 uM) | 0.25 | ul |
| RP_R (10 uM) | 0.25 | ul |
| RP_P (10 uM) | 0.15 | ul |
| TaqPath | 5 | ul |

2. Place 10 ul TaqPath/Primer Master Mix into each well for 3 controls and each patient sample.
3. Add 10 ul positive control (PC) to 1 well containing Master Mix. See below.
4. Add 10 ul extraction control (EC) to 1 well containing Master Mix. See below.
5. Add 10 ul nuclease-free water (NC) to 1 well containing Master Mix. See below.
6. Add 5-10 ul of each RNA sample (ensuring final volume of reaction is 20 μl) to well containing Master Mix. See below.
7. Seal the plate with Microfilm B.
8. Spin the plate in refrigerated centrifuge at 3000 RPM for 2 minutes at 4° C.

An Exemplary Sample Set Up is Depicted as Follows

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | | | | | | |
|---|---|---|---|---|---|---|---|
| A | PC | EC | S1 | S2 | . . . | | |
| B | | | | | | | |
| C | | | | | | | |
| . . . | | | | | | S93 | NC |

NC = no template control,
EC = extraction control,
PC = positive control,
S# = sample

qPCR Run
1. Place the plate into the qPCR machine.
2. Select the protocol “SARS-nCoV-2 test” from the Saved Files library.

| Stage | Temp | Time | | Repeat |
|---|---|---|---|---|
| 1 | 25° C. | 2 | minutes | X1 |
| 2 | 50° C. | 15 | minutes | X1 |
| 3 | 95° C. | 2 | minutes | X1 |
| 4, Step 1 | 95° C. | 3 | seconds | X45 |
| 4, Step 2 | 55° C. | 30 | seconds | |

3. Scan plate barcode.
4. Name Plate (Plate ID): <date-SARS-CoV-2 initials #>
5. Review information on screen and on the worksheet with a second technologist and confirm accuracy.
6. Tap RUN.

Data Analysis and Interpretation of Controls

TABLE 37
Expected Results

| | Expected Control Results | | |
|---|---|---|---|
| Type | N1 primer | N2 primer | RNase P |
| No template control (NC) | Neg, ≥40 Ct | Neg, ≥40 Ct | Neg, >40 Ct |
| Positive control (PC) | Pos, <40 Ct | Pos, <40 Ct | Neg, >40 Ct |
| Extraction control (EC) | Neg, ≥40 Ct | Neg, ≥40 Ct | Pos, ≤35 Ct |

Sample Results and Interpretation
1. If NC is positive for N1, N2, or RP, the PCR reagents may be contaminated. Repeat PCR for the entire plate with new PCR reagents.
2. If positive control is negative for N1 or N2 or both, the PCR may have failed. Repeat PCR for the entire plate.
3. If extraction control is positive for N1 or N2 AND there are N1 or N2 positive samples on the plate, the extraction reagents may be contaminated. Repeat extraction and PCR with new extraction reagents for EC and samples with positive results.
4. If extraction control is negative for N1 and N2 and neg or indeterminate for RP AND there are samples with negative or indeterminate RP on the plate, the extraction may have failed. Re-extract and repeat PCR for EC and samples with RP>35 Ct.

TABLE 38
Interpretation of sample results.

| N1 | N2 | RNase P | Interpretation | Report |
|---|---|---|---|---|
| Pos (<40 Ct) | Pos (<40 Ct) | Pos (<35 Ct), OR | Detected | Positive |
| | | Ind (35 up to 40 Ct), | | |
| | | OR | | |
| | | Neg (≥40 Ct) | | |
| Neg (≥40 Ct) | Neg (≥40 Ct) | Pos (<35 Ct) | Not detected | Negative |
| Neg (≥40 Ct) | Neg (≥40 Ct) | Ind (35 up to 40 Ct) | Indeterminate; | Indeterminate |
| | | | Repeat extraction and | |
| | | | retest. If the result is | |
| | | | unchanged, report as | |
| | | | inconclusive and | |
| | | | request another sample | |
| | | | if clinically indicated | |
| Neg (≥40 Ct) | Neg (≥40 Ct) | Neg, ≥40 Ct | Invalid; | Invalid |
| | | | Repeat extraction and | |
| | | | retest. If the result is | |
| | | | unchanged, report as | |
| | | | invalid and request | |
| | | | another sample if | |
| | | | clinically indicated | |

| N1 or N2 Pos (<40 Ct), | Pos (<35 Ct), OR | Indeterminate; | Indeterminate |
|---|---|---|---|
| but not both | Ind (35 up to 40 Ct), | Repeat extraction and | |
| | OR | retest. If the result is | |
| | Neg (≥40 Ct) | unchanged, report as | |
| | | inconclusive and | |
| | | request another sample | |
| | | if clinically indicated | |

1. If the controls are expected as above and the sample are both positive for N1 and N2, the test is valid, regardless of whether the sample is positive for RNase P. The sample is positive.
2. If the controls are expected as above and the sample is negative for both N1 and N2, and positive for RNase P, the test is valid and the sample is negative.
3. If the controls are expected as above and the sample is negative for both N1 and N2, and indeterminant for RNase P, the test is Indeterminate. Repeat from extraction and qPCR.
4. If the controls are expected as above and the sample is negative for both N1 and N2, but the sample is also negative for RNase P, the test is invalid. Repeat from extraction and qPCR. If the sample remains invalid or is Indeterminate, request another sample.
5. If the controls are expected as above and the sample is positive for either N1 or N2 but not both, the test is Indeterminate. Repeat from extraction and qPCR.
6. To resolve indeterminate results, extracted RNA may be concentrated by decreasing elution volume at the discretion of the Laboratory Director.

Quality Control
Quality control of reagents including lot to lot testing will be performed per NYS CLEP standards, including for controls.
Include an extraction control sample with each plate extraction and add to PCR.
Include a positive control and NC in each PCR run.

Limitations
This test is a laboratory developed test that has been authorized by NYS under an Emergency Use Authorization (EUA) for use by authorized laboratories (fda.gov/emergency-preparedness-and-response/counterterrorism-and-emerging-threats/cornavirus-disease-2019-covid-19). This laboratory developed test is not approved by the FDA.
Performance of this test has only been evaluated in saliva samples placed into DRUL2 buffer. Testing in other sample types may result in inaccurate results.
Negative results do not preclude SARS-Cov-2 infection.
   Factors such as the optimal sample type or sample collection timing in the course of disease have not been evaluated. Multiple tests from the same patient may be required. [3]
   Mutations within the primer and/or probe binding regions of the viral genome could affect primer binding and therefore may result in failure to detect the presence of virus.
   Self-collection technique or transport conditions may affect the quality of the sample received.

Assay Performance Characteristics/Validation

1) Limit of Detection (LOD)

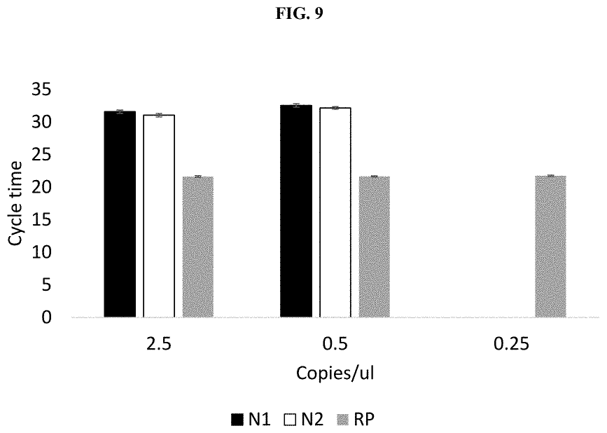

The specimen matrix was made using 5 pooled saliva samples and DRUL2 buffer at equal volumes spiked in triplicate with 10-fold serial dilutions of chemically-inactivated SARS-CoV-2 virus [Zeptometrix, NATtrol SARS-Related Coronavirus 2 (SARS-CoV-2) Stock, Cat #NATSARS(COV2)-ST)] as specified in the table below and extracted using the magnetic bead method. The sample was plated in triplicates at each concentration of RNA. The limit of detection (LOD) is 0.5 copy/ul. Viral RNA was detected in 100% of triplicate samples at the 0.5 copy/ul of virus (FIG. 9).

TABLE 39
Determination of LOD with DRUL2 and magnetic
bead extraction using Kingfisher Apex.

| RNA concentration (copy/ul) | 50 | 5 | 0.5 | 0.05 | 0 |
|---|---|---|---|---|---|
| Primer N1 Mean cycle time | 26.79 | 30.11 | 33.42 | 40.89 | 41.89 |
| Primer N1 Standard deviation | 0.08 | 0.07 | 0.92 | 1.22 | 0.10 |
| Primer N2 Mean cycle time | 25.64 | 28.95 | 34.29 | NA | NA |
| Primer N2 Standard deviation | 0.05 | 0.14 | 2.95 | NA | NA |

The LOD was confirmed with specimen matrices made with five pooled saliva samples, spiked with 2.5, 0.5 and 0.25 copies/ul of chemically modified viral RNA in DRUL2 buffer. RNA was individually extracted from 20 specimen matrices spiked at each of three viral RNA concentrations. A total of 60 samples were extracted. We confirmed that our LOD is 0.5 copies/ul in 20 of 20 saliva samples in DRUL2 buffer (Table 40).

TABLE 40
Confirmation of LOD with DRUL2 and magnetic
bead extraction using Kingfisher Apex.

| RNA concentration (copy/ul) | 2.5 | 0.5 | 0.25 |
|---|---|---|---|
| Positive/Tested | 20/20 | 20/20 | 7/20 |
| Primer N1 Mean cycle time | 31.59 | 32.55 | NA |
| Primer N1 Standard deviation | 0.25 | 0.27 | NA |
| Primer N2 Mean cycle time | 31.05 | 32.16 | NA |
| Primer N2 Standard deviation | 0.27 | 0.20 | NA |

2) Analytical Specificity

The RUCGL-SCV2 test uses primer probe sets with the same sequence as designed and used in the Triplex CII-SARS-CoV-2 rRT-PCR test EUA [1], which included its own specificity analysis. Thus, no separate analysis was performed.

The Triplex CII-SARS-CoV-2 rRT-PCR test EUA [1] analytical specificity studies are briefly provided herein as described. The Triplex CII-SARS-CoV-2 rRT-PCR assay primer and probe sets was tested in silico for potential cross-reactivity with sequences of other representative respiratory viral and bacterial pathogens listed below. Blastn analyses did not identify potential homologous regions between the primers/probes and the target genome sequences due to high number of mismatches in very short oligonucleotide or probe sequences. To further exclude potential homology, relaxed blastn parameters were used for re-analysis so that even very short alignments with weak matching scores could be identified. The N1 Forward and Reverse primers showed 91% and 89% homology, respectively, and the N1 probe showed 76% homology with SARSCoV-1 (2003), therefore, cross-reactivity of the N1 target with SARS-CoV-1 is not expected. The N2 Forward and Reverse primers showed 96% and 100% homology, respectively, and the N2 probe showed 85% homology with SARS-CoV-1 (2003). No other organism/viruses analyzed showed greater than 80% homology with any primers/probes from the Triplex CII-SARS-CoV-2 rRT-PCR assay.

### List of Viruses Analyzed in Silico for Cross-Reactivity.

| Name | Accession number | Name | Accession Number |
|---|---|---|---|
| Human coronavirus 229E | NC_002645.1 | Human rhinovirus 14 | NC_001490.1 |
| Snake adenovirus | NC_009989.1 | Human coronavirus OC43 | NC_006213.1 |
| Human enterovirus A | NC_001612.1 | Human adenovirus D | NC_010956.1 |
| Human coronavirus HKU1 | NC_006577.2 | Human rhinovirus 89 | NC_001617.1 |
| Rhizobium phage 16-3 | NC_011103.1 | Human coronavirus NL63 | NC_005831.2 |
| Bovine enterovirus | NC_001859.1 | Human adenovirus B2 | NC_011202.1 |
| SARS-coronavirus | NC_004718.3 | Poliovirus | NC_002058.3 |
| Human adenovirus B1 | NC_011203.1 | MERS-coronavirus | NC_019843.3 |
| Porcine teschovirus 1 | NC_003985.1 | Murine adenovirus 3 | NC_012584.1 |
| Human Metapneumovirus (hMPV) | NC_039199.1 | Porcine sapelovirus 1 | NC_003987.1 |
| Cercopithecine herpesvirus 5 strain 2715 NC_012783.2 | | Parainfluenza virus 1 | NC_003461.1 |
| Simian enterovirus A | NC_003988.1 | Human adenovirus 54 | NC_012959.1 |
| Parainfluenza virus 2 | NC_003443.1 | Porcine enterovirus 9 | NC_004441.1 |
| | | strain UKG/410/7 3 polyprotein gene | |
| Turkey adenovirus 1 | NC_014564.2 | Parainfluenza virus 3 | NC_001796.2 |
| Avian sapelovirus | NC_006553.1 | Murine adenovirus 2 | NC_014899.1 |
| Parainfluenza virus 4 | NC_021928.1 | Possum enterovirus | W1 NC_008714.1 |
| Fowl adenovirus E | NC_014969.1 | | |
| Influenza A virus (A/New | NC_007366.1 | | |
| York/392/2004(H3N2) | NC_007367.1 | | |
| | NC_007368.1 | | |
| | NC_007369.1 | | |
| | NC_007370.1 | | |
| | NC_007371.1 | | |
| | NC_007372.1 | | |
| | NC_007373.1 | Japanese eel endothelial cells | |
| Human rhinovirus C | NC_009996.1 | infecting virus | NC_015123.1 |
| Enterovirus J strain 1631 | NC_010415.1 | Simian adenovirus 49 | NC_015225.1 |
| Enterovirus J strain N203 | NC_013695.1 | Fowl adenovirus C | NC_015323.1 |
| Enterovirus F strain BEV-261 polyprotein | | | |
| gene, complete | NC_021220.1 | Raptor adenovirusA | NC_015455.1 |
| Enterovirus sp. Isolate CPML_810 9/08NC_024073.1 | | Bat adenovirus 2 | NC_015932.1 |
| Yak enterovirus strain SWUNAB001 NC_029854.1 | | South polar skua adenovirus-1 NC_016437.1 | |
| Enterovirus SEV-gx | NC_029905.1 | Bat adenovirus TJM | NC_016895.1 |
| Human enterovirus strain V13-0285, | | | |
| partial genome | NC_030454.1 | Chimpanzee adenovirus Y25 NC_017825.1 | |
| Influenza A virus | NC_007375.1 | | |
| (A/Korea/426/1968(H2N2)) | NC_007376.1 | | |
| | NC_007377.1 | | |
| | NC_007378.1 | | |
| | NC_007379.1 | | |
| | NC_007380.1 | | |
| | NC_007381.1 | | |
| | NC_007382.1 | | |
| | NC_007383.1 | | |
| Ovine adenovirus A | AC_000001.1 | Goose adenovirus 4 | NC_017979.1 |
| Bovine adenovirus B | AC_000002.1 | Bovine adenovirus 6 strain 671130 NC_020074.1 | |
| Canine adenovirus 1 | AC_000003.1 | Simian adenovirus 20 strain | |
| | | ATCC VR-541 | NC_020485.1 |
| Duck adenovirus A | AC_000004.1 | Titi monkey adenovirus ECC-2011 | |
| NC_020487.1 | | | |
| Human Mastadeno virus A | AC_000005.1 | Simian adenovirus C isolate BaAdV-2 | |
| NC_021168.1 | | | |
| Human adenovirus D | AC_000006.1 | Fowl adenovirus 5 strain 340 | |
| NC_021221.1 | | | |
| Human adenovirus 2 | AC_000007.1 | Pandoravirus dulcis NC_021858.1 | |
| Human adenovirus 5 | AC_000008.1 | Simian adenovirus 18 NC_022266.1 | |
| Porcine adenovirus C | AC_000009.1 | Turkey adenovirus 4 isolate TNI1 NC_022612.1 | |
| Influenza A virus (A/Puerto | NC_002016.1 | Simian adenovirus 21 AC_000010.1 | |
| Rico/8/1934(H1N1)) | NC_002017.1 | Turkey adenovirus 5 isolate1277BT | |
| NC_022613.1 | | | |
| | NC_002018.1 | Simian adenovirus 25 AC_000011.1 | |
| | NC_002019.1 | Ralstonia phage RSK1 DNA NC_022915.1 | |
| | NC_002020.1 | Murine adenovirus A AC_000012.1 | |
| | NC_002021.1 | California sea lion adenovirus 1 strain Zc11- | |
| 030NC_024150.1 | | | |
| | NC_002022.1 | Fowl adenovirus D AC_000013.1 | |
| | NC_002023.1 | Pigeon adenovirus 1completegenome, strainIDA4 | |
| NC_024474.1 | | | |
| Fowl adenovirus A | AC_000014.1 | Duck adenovirus 2 strain GR | |
| NC_024486.1 | | | |
| Turkey adenovirus A | AC_000016.1 | Lizard adenovirus 2 isolate 23-06 | |
| NC_024684.1 | | | |
| Human adenovirus type 1 | AC_000017.1 | Simian adenovirus DM-2014isolate23336 | |
| NC_025678.1 | | | |
| Human adenovirus type 7 | AC_000018.1 | Psittacine adenovirus 3 isolateHKU/Parrot19 | |
| NC_025962.1 | | | |
| Influenza B virus | NC_002204.1 | Human Adenovirus type 35 AC_000019.1 | |
| (B/Lee/1940) | NC_002205.1 Equine adenovirus 2 isolate EAdV2.385/75.9 | | |
| NC_027705.1 | | | |
| | NC_002206.1 | Canine adenovirus type 2 AC_000020.1 | |
| | NC_002207.1 | Skunk adenovirus PB1 NC_027708.1 | |
| | NC_002208.1 | Porcine adenovirus 3 AC_000189.1 | |
| | NC_002209.1 | Simian adenovirus 13 strain P-9 NC_028103.1 | |
| | NC_002210.1 | Tree shrew adenovirus1 AC_000190.1 | |
| | NC_002211.1 | Simian adenovirus16 strain C-8 NC_028105.1 | |
| Bovine adenovirus A | AC_000191.1 | Simian adenovirus 19 strainAA153 | |
| NC_028107.1 | | | |
| Fowl adenovirus D | NC_000899.1 | Simian adenovirus 8 strain P-5 | |
| NC_028113.1 | | | |
| Murine adenovirus A | NC_000942.1 | UnidentifiedadenovirusisolateCSPAdV_2 | |
| NC_030116.1 | | | |
| Human adenovirus C | NC_001405.1 TokyovirusA1 DNA, nearly complete genome NC_001405.1 | | |
| NC_030230.1 | | | |
| Respiratory syncytial virus | NC_001803.1 | Human adenovirus F | |
| NC_001454.1 | | | |
| Equine adenovirus1 strain M1 | NC_030792.1 | Chlamydia pneumonia | |
| NC_005043.1 | | | |
| Human adenovirus A | NC_001460.1 Pigeon adenovirus2 isolateYPDS- | | |
| YV1.A19.112013NC_031503.1 | | | |
| Haemophilus influenza | NZ_LN831 035 | Fowl adenovirus A | |
| NC_001720.1 | | | |
| Legionella pneumophila | NZ_LR134 380.1 | Canine adenovirus | |
| NC_001734.1 | | | |
| Mycobacterium tuberculosis | NC_000962.3 | Duck adenovirus A | |
| NC_001813.1 | | | |
| Streptococcus pneumonia | NZ_LN831 051.1 | Bovine adenovirus 3 complete | |
| genomeNC_001876.1 | | | |

Streptococcus pyogenes NZ_LN831 034 Hemorrhagic enteritis virus NC_001958.1
Bordetella pertussis NC_018518.1 Frog adenovirus 1 NC_002501.1
Mycoplasma pneumonia NZ_CP010 546.1 Bovine adenovirus type 2 NC_002513.1
Pneumocystis jirovecii (PJP) MK984200 Bovine adenovirus D NC_002685.2
Candida albicans NC_018046 Porcine adenovirus 5 NC_002702.1
Pseudomonas aeruginosa NC_002516.2 Human adenovirus E NC_003266.2

| Staphylococcus epidermidis | NZ_CP035 288.1 | Garlic virus A |
|---|---|---|
| NC_003375.1 | | |
| ASM609437v1 | NZ_CP035 289.1 | Ovine adenovirus 7 |
| NC_004037.2 | | |
| | NZ_CP035 290.1 | Bovine adenoassociated virus |
| NC_005889.1 | | |
| Streptococcus salivarius | NZ_LR134 274.1 | Simian adenovirus 3 |
| NC_006144.1 | | |
| Human enterovirus D | NC_001430.1 | Macacine herpesvirus 3 |
| NC_006150.1 | | |
| Human enterovirus B | NC_001472.1 | Simian adenovirus 1 |
| NC_006879.1 | | |

3) Comparative LOD

This assay is the second assay we have validated to detect SARS-CoV-2 in saliva specimens. The first assay was approved for clinical use by New York State Department of Health Wadsworth in Sep. 24, 2020. The first assay, which used a column-based extraction method (QIAgen QIAmp DSP Viral RNA Mini Kit) and the primer/probe sets validated by the CDC and the New York State Department of Health had a LOD of 1 copy/ul (Tables 10-13) as compared to this current assay which has a LOD of 0.5 copy/tit (Table 39, FIG. 9). Our current assay has a mean cycle time of 32.55 and 32.16 for N1 and N2 respectively at 0.5 copy/tit which is lower than what was seen at 2 copies/1d in our Sep. 24, 2020 assay, indicating that our current assay is more sensitive than our first (DRUL1) EUA approved assay (described in Example 2).

TABLE 41
Confirmation of LOD in DRUL buffer (first
assay) and column-based extraction

| RNA concentration (copy/ul) | 2 | 1 | 0.2 |
|---|---|---|---|
| Positive/Tested | 20/20 | 20/20 | 17/20 |
| Primer N1 Cycle time | 33.61 | 34.65 | NA |
| Primer N1 Standard deviation | 0.43 | 0.34 | NA |
| Primer N2 Cycle time | 36.57 | 37.67 | NA |
| Primer N2 Standard deviation | 0.32 | 0.73 | NA |

4) Clinical Evaluation

A clinical evaluation study was conducted as follows using contrived positive specimens. Forty-four negative and 44 samples spiked with chemically-inactivated SARS-CoV-2 virus [Zeptometrix] were individually extracted and tested. Of the 44 spiked samples, 34 were spiked with 0.5 copies/1d and 10 were spiked with 2.5 copies/ul. Seven of the 88 samples required re-extraction and PCR due to initial Indeterminate results. All seven samples were evaluable upon repeat testing. All negative samples tested negative. Forty-three of 44 spiked samples were positive (98% sensitivity). The results are shown in Table 42.

TABLE 42
Clinical Evaluation.

| | | Weak | |
|---|---|---|---|
| | Negative | Positive | Positive |
| RNA concentration (copy/ul) | 0 | 0.5 | 2.5 |
| Positive/Tested (% positive) | 0/44 | 33/34 (97%) | 10/10 (100%) |
| Repeated due to initial | 3/44 | 2/34 | 2/10 |
| inconclusive result/Tested | | | |
| Primer N1 Mean cycle time | NA | 32.74 | 32.60 |
| Primer N1 Standard deviation | NA | 0.76 | 0.46 |
| Primer N2 Mean cycle time | NA | 32.57 | 32.22 |
| Primer N2 Standard deviation | NA | 1.43 | 1.18 |

Summary Statement

We have developed real-time reverse transcriptase polymerase chain reaction (RT-PCR) test for the qualitative detection of RNA from the SARS-CoV-2 in patient saliva. This assay uses a special preparation of reagents to stabilize and inactivate SARS-CoV-2 virus, to stabilize its RNA, and uses inexpensive reagents with long half-life at room temperature, all aspects of providing a new inexpensive, utilitarian and safe means by which SARS-CoV-2 testing can be done by individuals, at home, in the office, or elsewhere.

We go on to demonstrate a strategy and detailed protocol for quantifying this RNA from saliva. This provides a unified protocol that works in thousands of assays. Key unique aspects of the protocol involve development of a lysis buffer (DRUL2) that lysis virus, stabilizes RNA, and prepares samples for scalable purification assays involving magnetic beads. Moreover, unique and temporally ordered steps have been added to convert samples collected into uniformly thin liquids that can be drawn up at scale by robotic liquid handlers, a critical component of scaling. These steps include heating samples prior to opening tubes, which further inactivates any remaining virus in shipping bags or the outside of sample tubes, and, together with DTT subsequently added, thins saliva prior to robotic liquid handling.

Finally, we have created a unique binding buffer to which the saliva/DRUL2/DTT mixture is added. We have optimized this buffer to maximize binding of SARS-CoV-2 RNA from crude saliva to allow its efficient capture by magnetic beads. We have optimized this protocol for use with carboxylated SPRI beads, which are readily available at low cost commercially or can be generated within the laboratory.

This entire protocol has been validated as per as per the Wadsworth Validation Procedure for SARS-CoV-2 LDT. The assay was validated for accuracy of identification of SARS-CoV-2 using the Columbia University EUA primers. The assay is accurate and precise. The assay is extremely sensitive, with an LOD of 0.5 copy/ul of SARS-CoV-2 RNA detected from chemically inactivated virus (from Zeptometrix). This assay is being used for qualitative detection of SARS-CoV-2 RNA in patient saliva submitted to the Rockefeller University Clinical Genomics Laboratory (RUCGL).

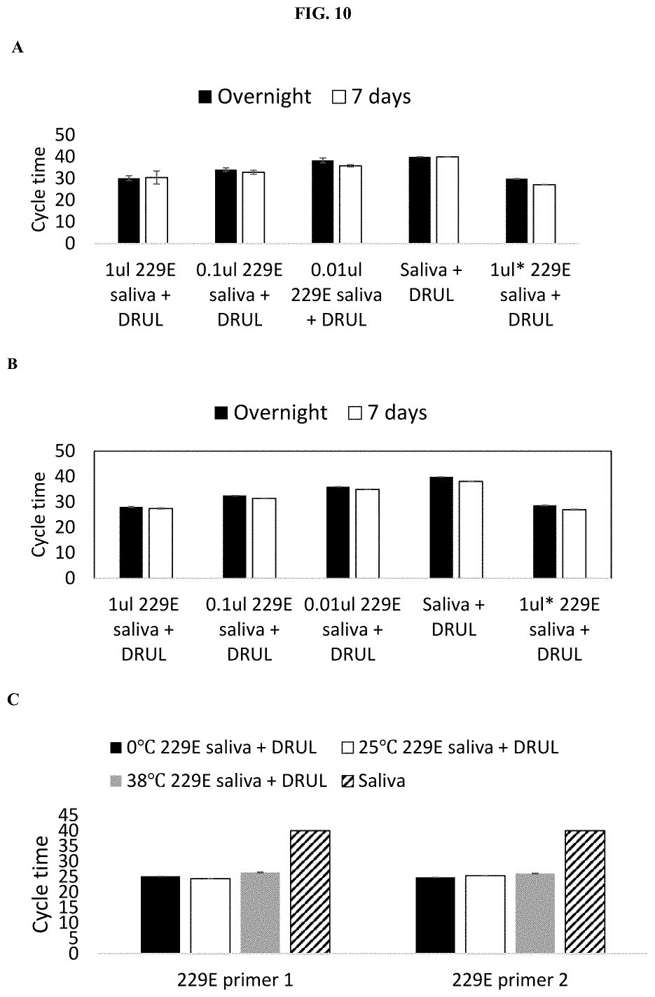

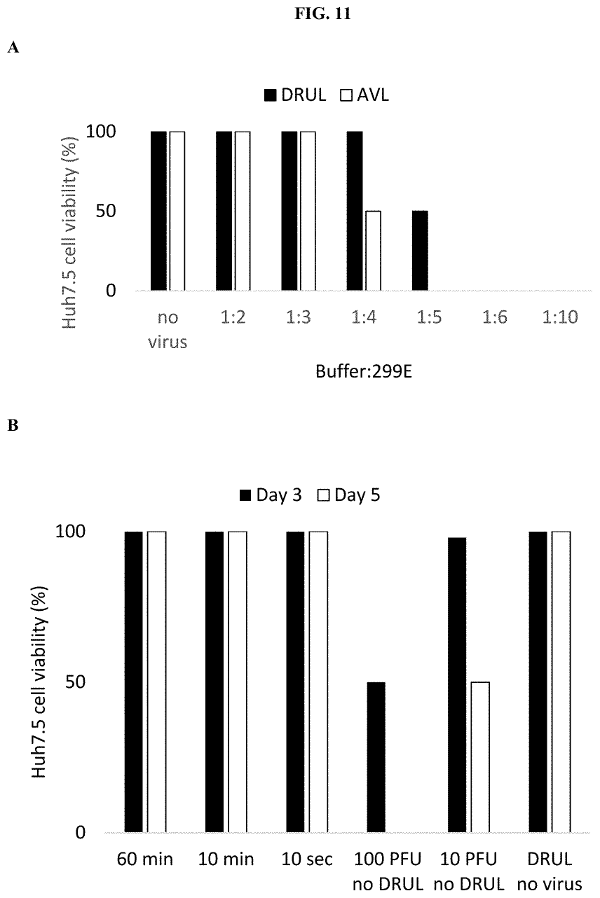

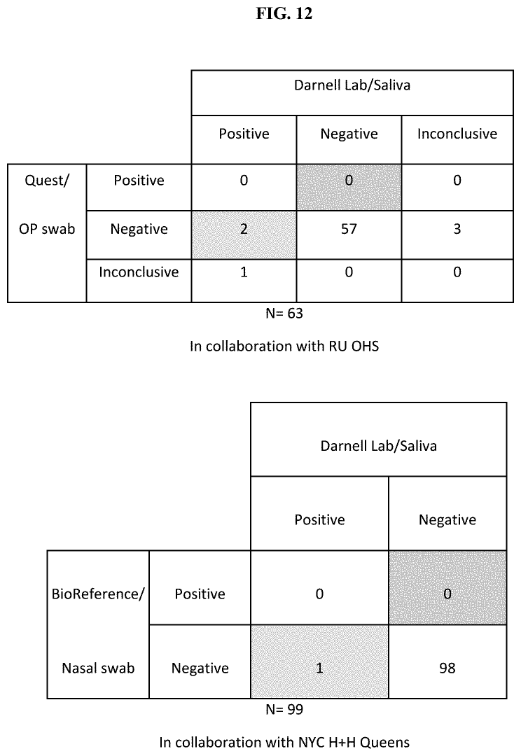

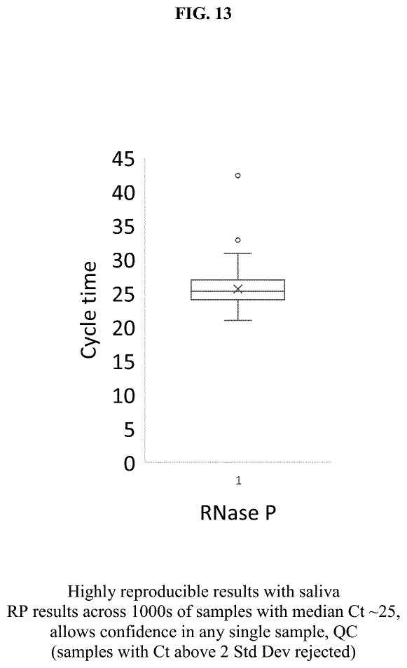

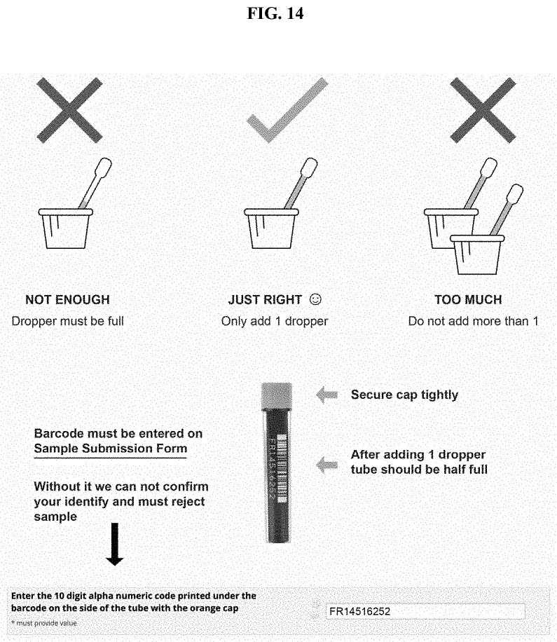

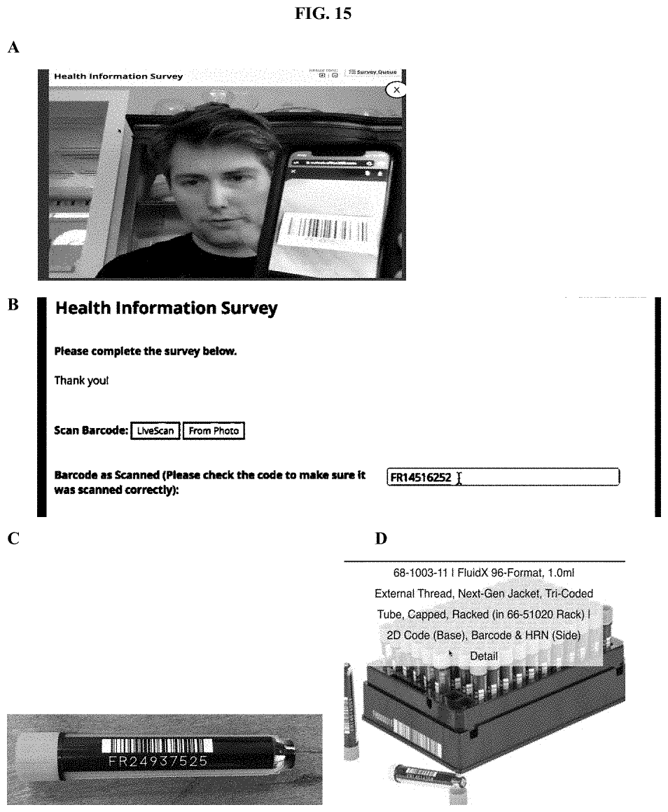

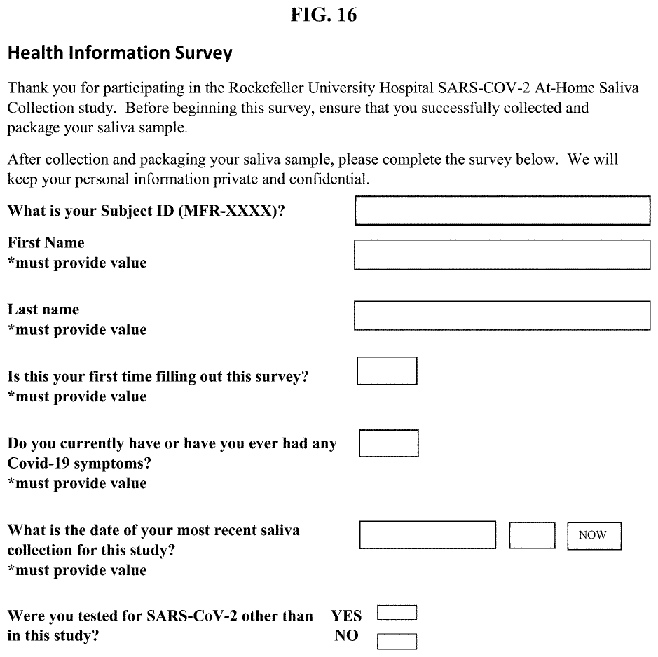

As noted herein, the protocol, system, methods, buffers utilized in the DRUL assays provides several significant and important advantages. RNA is very stable in the DRUL/DRUL2 buffers and samples can be stably left at room temperature for many days, including 7 days, a week with RNA remaining stable for satisfactory isolation and maintained testing sensitivity (FIG. 10). DRUL/DRUL2 buffer rapidly and effectively inactivates virus, including as demonstrated in FIG. 11. The test sensitivity is remarkable and improved/better than other tests currently available (FIG. 12). Test reproducibility and ability to detect positives missed in other tests in a direct comparison are demonstrated in FIG. 13. Details of sample collection and labeling, including an implemented bar code system for sample labeling are provided in FIGS. 14-16.

### REFERENCES

[1] Accelerated Emergency Use Authorization (EUA) Summary, The Triplex Cii-SARS-CoV-2 rRT-PCR Test (Columbia University Laboratory of Personalized Genomic Medicine)—available at fda.gov/media/137983/download.
[2] Chomczynski P & Sacchi N. (1987). “Single-step method of RNA isolation by acid guanidinium thiocyanate-phenol-chloroform extraction”. Anal. Biochem. 162: 156-159. doi:10.1016/0003-2697(87)90021-2. PMID 2440339.
[3] To K K, Tsang O T, Chik-Yan Yip C, Chan K H, Wu T C, Chan J M C, Leung W S, Chik T S, Choi C Y, Kandamby D H, Lung D C, Tam A R, Poon R W, Fung A Y, Hung I F, Cheng V C, Chan J F, Yuen K Y. Consistent detection of 2019 novel coronavirus in saliva. Clin Infect Dis. 2020 Feb. 12. pii: ciaa149. doi: 10.1093/cid/ciaa149.

### Example 4

### Standard Procedure—Specimen Receipt and Handling

Rockefeller University—Clinical Genomics Laboratory

### SOP #17

Specimen Receipt and Handling (SARS-CoV-2 testing)

### Version 1

1. Purpose

This procedure is intended to guide in the collection, shipment, receipt, accessioning, and handling of saliva specimens sent to or from the Rockefeller University Clinical Genomics Laboratory (RUCGL) for clinical testing for SARSCoV-2 testing. Specimens must be collected and shipped in accordance with approved procedures to ensure extracted nucleic acids will be of the highest possible quality for downstream processing. Upon receipt in the clinical laboratory, specimens are evaluated based on specific acceptance and rejection requirements. Acceptable specimens are assigned a unique accession number for accurate tracking throughout all laboratory processes.

2. Affected Personnel
2.1. This procedure applies to all RUCGL personnel.

3. Acceptable Specimen Types
3.1. Saliva samples in DRUL buffer or PrimeStore MTM.
3.2. Saliva samples in DRUL2 buffer.

4. Specimen Collection, Storage, and Transport Requirements
4.1. Saliva for viral RNA must be collected in DRUL buffer vial, PrimeStore MTM, or DRUL2 filled with 300 ul saliva collected according to patient instruction sheet.
4.2. Samples must be labeled with two unique identifiers such as name, date of birth, medical record number or barcode associated with at least two unique identifiers and a sample collection date and time.
4.3. Samples must be accompanied by a completed requisition form or patient demographic data and date/time of collection in a Redcap record. The information listed on the requisition form must match the information on the specimen.
4.4. Specimens must be placed in drop box or packaged according to IATA guidelines for shipping. Appropriate packaging and shipping materials will be provided to requisitioners upon request.

5. Specimen Acceptability and Rejection Criteria
5.1. Acceptance Criteria
5.1.1. Legibly labeled with at least 2 unique identifiers or barcode associated with at least 2 unique identifiers
5.1.2. Shipped in appropriate conditions
5.1.3. Received in intact container
5.1.4. If specimen is irreplaceable and do not meet the criteria below, acceptance is at the discretion of the Clinical Laboratory Director.
5.1.5. Received in the DRUL1, DRUL2, or PrimeStore MTM vial
5.1.6. Filled with 300 ul of saliva
5.2. Reject any specimen not meeting the above criteria

6. Materials and Equipment
6.1. Computer with access to the clinical share drive on box

7. Procedure
7.1. Specimens are delivered to Smith Hall Room 313B. Alternatively, they are shipped overnight by Federal Express to:

### Clinical Genomics Laboratory

### Rockefeller University

### 1230 York Avenue

### New York, N.Y. 10065

7.2. Open the RUCGL Accessioning Log in the shared drive.
7.3. With gloved hands, review the requisition form accompanying the specimens.

The following elements must be completed:
7.3.1. Patient name
7.3.2. Medical record number
7.3.3. Date of birth
7.3.4. Date of specimen collection
7.3.5. Type of specimen
7.3.6. Requesting clinician's name
7.3.7. Indication for testing/clinical diagnosis
7.3.8. Test requested
7.4. Inspect contents of shipment or delivery and any supporting documentation and record following on bottom of requisition form:
7.4.1. Date received
7.4.2. Time received
7.4.3. Container/specimen type received
7.4.4. Specimen volume/quantity (estimated)
7.5. Assign an Accession number to the specimen

Each sample for viral RNA detection is assigned the two-digit year of receipt, a three-digit number designating the individual and the number of times a sample is received from this same patient:
20-S001-1 (first sample from patient 001)
20-S001-2 (second sample from patient 001)
20-S002-1 (first sample from patient 002)
7.6. Enter the patient information from the requisition into the Accessioning Log and add the assigned Accession number.
7.7. Confirm that specimens meet acceptability criteria
7.7.1. Consult Laboratory Director as necessary if any questions arise regarding acceptability.
7.7.2. Promptly notify the requisitioner if specimens are deemed unacceptable.
7.7.3. Note the reasons for sample rejection and the date, time, and to whom the notification was made.
7.8. Store specimens at room temperature until ready to assay.
7.9. When referring samples to another clinical laboratory:
7.9.1. Confirm that the laboratory is a New York State permitted laboratory. Alternatively, if the test is not available from a New York State permitted laboratory, submit a Non-Permitted Laboratory Request.
7.9.2. Record the name of the laboratory, date the specimen was sent, date the results were reported, and the date the report was received in the Accession Log.
7.10. When receiving samples from another clinical laboratory:
7.10.1. Provide the Instructions Manual to the clinical laboratory.

8. Order Entry Verification
8.1. At the beginning of each month, the Clinical Laboratory Technologist will conduct an audit of up to 5 cases from the previous month to verify the completeness of the Accessioning Log.

9. Safety Precautions
9.1. Lab coats and gloves must be used when handling specimens.

10. Computer Downtime Procedure
10.1. In the event of a power failure, computer failure or other, accession using the Temporary Accessioning Form, using a temporary sample ID. When access to the Accessioning Log is restored, transfer the information. Scan and retain the Temporary Accessioning Form in the clinical shared drive.

### Example 5

### Standard Procedure—Preparing Primers and Probes

### Rockefeller University—Clinical Genomics Laboratory

### SOP #16

### Preparing Multiplex SARS-CoV-2 Primers/Probes

### Version 1

1. Purpose of the Procedure

The purpose of this procedure is to prepare primers/probes for use in the SARS-CoV-2 version 2 assay for testing saliva using real-time reverse transcriptase quantitative PCR.

2. Principle of the Procedure

Primer/probes are diluted, combined and aliquoted in volumes needed for half and whole PCR plates. This results in less variability when making master mix and reduces freeze thaw cycles.

3. Personnel Affected

This procedure applies to the Rockefeller University Clinical Genomics Laboratory (RUCGL) clinical laboratory director, supervisor, and clinical technologists.

4. Reagents and Quality Control

All of the following reagents are stored at room temperature unless otherwise specified and must be discarded after 1 year of receipt.
4.1. Primers and Probes, LGC Biosearch Technologies, Custom, Store at −80 C as specified in the Triplex CII-SARS-CoV-2 rRT-PCR TEST (Columbia University Laboratory of Personalized Genomic Medicine) EUA
4.1.1. SARS-CoV-2 forward primer for N1 target (nCoV_NF1)
4.1.2. SARS-CoV-2 reverse primer for N1 target (nCoV_NR1)
4.1.3. SARS-Cov-2 FAM Probe for N1 target (nCoV_NP1)
4.1.4. SARS-CoV-2 forward primer for N2 target (nCoV_NF2)
4.1.5. SARS-CoV-2 reverse primer for N2 target (nCoV_NR2)
4.1.6. SARS-CoV-2 Quasar 670 Probe for N2 target (nCoV_NP2)
4.1.7. RNAse P forward primer (RP_F)
4.1.8. RNAse P reverse primer (RP_R)
4.1.9. RNAse P CAL Fluor Orange 560 probe (RP_P)
4.2. Nuclease-free water

5. Consumables
5.1. Aerosol barrier pipette tips (DNase/RNase free)
5.2. PCR tubes
5.3. Disposable gloves, gowns, masks

6. Equipment
6.1. Pipettes P10, P20, P200

7. Procedure
7.1. Make 100 uM stock
7.1.1. Briefly spin each oligo tube.
7.1.2. Add nuclease-free water to each tube: Volume nuclease-free water in ul=“total nmol amount”×10
7.1.3. Vortex for 30 seconds until no particles are visible.
7.1.4. Aliquot the following into 78 ul aliquots and store in dark box labeled Multiplex 100 uM stock at −80 C.
7.1.4.1. SARS-CoV-2 forward primer for N1 target (nCoV_NF1)
7.1.4.2. SARS-CoV-2 reverse primer for N1 target (nCoV_NR1)
7.1.4.3. SARS-CoV-2 forward primer for N2 target (nCoV_NF2)
7.1.4.4. SARS-CoV-2 reverse primer for N2 target (nCoV_NR2)
7.1.5. Aliquot the following into 85 ul aliquots and store in dark box labeled Multiplex 100 uM stock at −80 C.
7.1.5.1. SARS-Cov-2 FAM Probe for N1 target (nCoV_NP1)
7.1.5.2. SARS-CoV-2 Quasar 670 Probe for N2 target (nCoV_NP2)
7.1.6. Aliquot the following into 30 ul aliquots and store in dark box labeled Multiplex 100 uM stock at −80 C.
7.1.6.1. RNAse P forward primer (RP_F)
7.1.6.2. RNAse P reverse primer (RP_R)
7.1.7. Aliquot the following into 18 ul aliquots and store in dark box labeled Multiplex 100 uM stock at −80 C.
7.1.7.1. RNAse P CAL Fluor Orange 560 probe (RP_P)
7.2. Make Master Primer Mix
7.2.1. Thaw one of each vial.
7.2.2. Vortex.
7.2.3. Combine contents in volumes below:

| | Volume per | Volume per 1000 |
|---|---|---|
| Reagent | sample (ul) | samples (ul) |
| nCoV_NF1 (100 uM) | 0.075 | 85 |
| nCoV_NR1 (100 uM) | 0.075 | 85 |
| nCoV_NP1 (100 uM) | 0.06 | 70 |
| nCoV_NF2 (100 uM) | 0.075 | 85 |
| nCoV_NR2 (100 uM) | 0.075 | 85 |
| nCoV_NP2 (100 uM) | 0.06 | 70 |
| RP_F (100 uM) | 0.025 | 30 |
| RP_R (100 uM) | 0.025 | 30 |
| RP_P (100 uM) | 0.015 | 18 |
| Nuclease-free water | 4.515 | 4966.5 |
| Total vol | 5 | 5500 |

7.2.4. Vortex.
7.2.5. Make 8×0.5 ml (for a full plate) and 8×0.25 ml (for half a plate) aliquots.
7.2.6. Label with lot #: MPM-date.
7.2.7. Store in dark box at −80 C.
7.2.8. Vials are ready for use.

### Example 6

### Standard Procedure—Sars-Ncov-2 Testing of Home Collected Saliva

### Rockefeller University—Clinical Genomics Laboratory

### SOP #15

SARS-nCoV-2 testing of home collected saliva, Version 2 (APEX)

### Version 1

1. Purpose of the Procedure

The purpose of this procedure is for the detection of nucleic acids from SARS-nCoV-2 in saliva samples collected at home and placed in lysis/RNA stabilization buffer by the patient using real-time reverse transcriptase quantitative PCR.

2. Principle of the Procedure

RNA isolated from saliva samples collected and stabilized at the point of collection is purified and reverse transcribed into cDNA. cDNA is amplified using multiplexed primers and probes that have been validated by the Triplex CII-SARS-CoV-2 rRT-PCR TEST (Columbia University Laboratory of Personalized Genomic Medicine) EUA with real-time PCR methodology. In the qPCR assay, the probe is hydrolyzed during amplification of the primer specific region, cleaving and releasing the fluorophore from the quencher, resulting in a fluorescence signal proportional to the target sequence present in the sample.

3. Personnel Affected

This procedure applies to the Rockefeller University Clinical Genomics Laboratory (RUCGL) clinical laboratory director, supervisor, and clinical technologists.

4. Accepted Specimen Types

Saliva samples collected at home and placed in supplied buffer (DRUL2) at point of collection using the RUCGL Saliva Collection Kit or similar. The Saliva Collection Kit includes a vial containing 300 ul of DRUL2. 300 ul of saliva sample collected in a disposable paper cup or other clean container is aliquoted into the buffer using a bulb syringe provided. The vial is tightly capped and inverted 10 times to mix the contents then delivered to the RU CGL. Details for collection are provided with the SARS CoV-2 RUCGL sample collection kit.

See SOP #17 and Example 4 Specimen Receipt and Handling

5. Specimen Acceptability and Rejection Criteria

See SOP #1 and Example 6 Specimen Receipt and Handling

6. Reagents and Quality Control

All of the following reagents are stored at room temperature unless otherwise specified and must be discarded after 1 year of receipt.
6.1. Guanidine Thiocyanate, Molecular Biology Grade 99% min, Fisher Cat #BP221-1, protect from light.
6.2. Sarkosyl, Molecular Biology Grade, Fisher Cat #234-500
6.3. 3M Sodium acetate, pH 5.5 (0.2 micron filtered), Invitrogen Cat #AM9740
6.4. Nuclease-free Water (0.2 micron filtered), Ambion Cat #AM9932 or equivalent
6.5. Dithiothreitrol (DTT), Fisher Scientific, Cat #R0861, Store at 2-8° C.
6.6. CleanNGS, 500 ml, Bulldog Bio, Cat #CNGS-0500, Store at 2-8° C.
6.7. Koptec Pure Ethanol, 200 proof, Decon Labs/ThermoFisher, Cat #V1016
6.8. PEG 8000, Promega, Cat #V3011
6.9. 5M NaCl, Invitrogen, Cat #Am9759
6.10. 0.1M Trisodium Citrate, Sigma, Cat #51804
6.11. Tween 20, Sigma, Cat #P1379
6.12. 100 mM HCl, Fisher Scientific, A508-P500
6.13. TaqPath 1 Step RT-qPCR, Life Tech Cat #A15300, Store at −20° C.
6.14. Quantitative synthetic RNA from SARS-Related Coronavirus 2, BEI Resources, Cat #NR-52358, Store at −20° C.
6.15. Primers and Probes, LGC Biosearch Technologies, Custom, Store at −80 C as specified in the Triplex CII-SARS-CoV-2 rRT-PCR TEST (Columbia University Laboratory of Personalized Genomic Medicine) EUA. NOTE: A master primer mix is prepared and frozen. Thaw 1 vial for plating (see SOP #16: Preparing Multiplex SARS-CoV-2 Primers/Probes)
6.15.1. SARS-CoV-2 forward primer for N1 target (nCoV_NF1)
6.15.2. SARS-CoV-2 reverse primer for N1 target (nCoV_NR1)
6.15.3. SARS-Cov-2 FAM Probe for N1 target (nCoV_NP1)
6.15.4. SARS-CoV-2 forward primer for N2 target (nCoV_NF2)
6.15.5. SARS-CoV-2 reverse primer for N2 target (nCoV_NR2)
6.15.6. SARS-CoV-2 Quasar 670 Probe for N2 target (nCoV_NP2)
6.15.7. RNAse P forward primer (RP_F)
6.15.8. RNAse P reverse primer (RP_R)
6.15.9. RNAse P CAL Fluor Orange 560 probe (RP_P)
6.16. HeLa cells (1.25×105 cells in 0.8 ml), see SOP #14 HeLa Extraction Control, Store at −80 C

7. Consumables
7.1. Aerosol barrier pipette tips (DNase/RNase free)
7.2. ThermoFisher KingFisher 96 well deep well plates, Cat #95040450
7.3. ThermoFisher 0.1 ml PCR reaction plates (96 well plates), Cat #4366932
7.4. BioRad Microseal B PCR plate seals, Cat #MSB 1001
7.5. Disposable gloves, gowns, masks

8. Reagent Preparation
8.1. Make 100 ml of DRUL2:

| Reagent | Amount needed | | Final Concentration |
|---|---|---|---|
| Guanidine Thiocyanate | 59.08 | g | 5M |
| 10% Sarkosyl | 5 | ml | 0.5% |
| 3M Sodium Acetate, pH 5.5 | 5.81 | ml | 175 mM |

| | |
|---|---|
| Nuclease-free Water | Bring up to 100 ml |

8.1.1. Add 300 ul to each sample collection vial.
8.1.2. Store at room temperature.
8.1.3. Wrap stock bottle in foil.
8.1.4. Label with lot #: DRUL2-date
8.2. Make 100 ml of binding buffer:

| Reagent | Amount needed | | Final Concentration | |
|---|---|---|---|---|
| PEG 8000 | 20 | g | 20% | (w/v) |
| 5M NaCl | 50 | ml | 2.5M | |
| 0.1M Trisodium Citrate | 10 | ml | 1 mM | |
| Tween 20 | 0.050 | ml | 0.05% | (v/v) |

| | | | |
|---|---|---|---|
| 0.1N HCl | 0.056 | ml | 0.56 mM |

| | |
|---|---|
| Nuclease-free Water | Bring up to 100 ml |

8.2.1. Make up to 1 L at a time.
8.2.2. Store at room temperature.
8.2.3. Label with lot #: BB-date
8.3. Make wash buffer, fresh each day:

| Reagent | Amount needed |
|---|---|
| Ethanol | 80 ml |
| Nuclease-free Water | Bring up to 100 ml |

8.4. Make 3M DTT stock solution:

| Reagent | Amount needed |
|---|---|
| DTT powder | 25 g |
| Nuclease-free Water | Bring up to 55.5 ml |

8.4.1. Make 1.6 ml aliquots and store at −20 C. Protect from light.
8.4.2. Label with lot #: DTT-date.
8.4.3. Thaw 1 vial per plate.
8.5. Make working stock of positive control:
8.5.1. For 100× stock
8.5.1.1. Put 1 ul of original vial into 289 ul of nuclease-free water.
8.5.1.2. Make 2 ul aliquots.
8.5.1.3. Store at −80 C.
8.5.2. Day of assay
8.5.2.1. Thaw 1 vial of 100× stock.
8.5.2.2. Put 1 ul of 100× stock into 99 ul of nuclease-free water.
8.5.2.3. Use as positive control in PCR plate.
8.5.2.4. Discard remainder.
8.6. Prepare HeLa cells for extraction control (see SOP #14 HeLa Extraction Control, step 8)
8.6.1. To a vial of HeLa, add 0.8 ml of DRUL2 to frozen pellet of HeLa cells.
8.6.2. Vortex and agitate until thawed and let sit for at least one hour and maximum one week at 25° C. before proceeding to extract RNA.
8.6.3. Label the vial with the date of thaw.
8.6.4. Use 200 ul as input volume for each extraction (24,000 cells).

9. Equipment
9.1. Applied Biosystems 7500Fast Dx Real-Time PCR Instrument
9.1.1. S/N 275031622
9.1.2. S/N 275031623
9.2. Thermofisher Kingfisher Apex
9.2.1. S/N 714-800104
9.2.2. S/N 714-DEM11
9.3. Brooks Fluid IntelliXcap 96-Format Automated Sample Tube Capper/Decapper, Model #46-8012, S/N 0334
9.4. Eppendorf Minicentrifuge, Model #5424, S/N 0006020
9.5. Eppendorf Centrifuge 5910 R (refrigerated), S/N 5942IG501511
9.6. Denver Instruments Balance, Model #XP-3000, S/N X066041
9.7. Pipettes P10, P20, P200

10. Controls

| Type | Reagent | Purpose | Include in |
|---|---|---|---|
| Negative | Nuclease-free water | Detect reagent | Each qPCR |
| control (NC) | | contamination | run |
| Positive | Quantitative synthetic | Detect failure | Each qPCR |
| control (PC) | RNA from SARS-Related | of run | run |
| | Coronavirus 2 | | |
| Extraction | Frozen HeLa cells | Detect reagent | Each extrac- |
| control (EC) | | contamination | tion batch |

11. Procedure
11.1.1. Heat all sample tubes in kits at 65° C.; for 15 minutes in dry oven.
11.1.2. Place tubes in 96 well rack and spin at 2000×g RT for 2 minutes.
11.1.3. Place in decapper to remove caps.
11.1.4. Add 16 ul of 3M DTT stock to each 600 ul sample (saliva+DRUL2).
11.1.5. Recap tubes and vortex the tubes in the rack.
11.1.6. Decap tubes and remove 200 ul from each sample tube and add to a well in the 96 well deep well plate. Recap the remaining sample for storage.
11.1.7. Swirl the Binding Buffer to mix the viscous solution then add 165 ul to each sample well. Mix gently so as not to introduce bubbles.
11.1.8. Thoroughly mix bead stock solution to disperse beads by swirling and inverting, then add 110 ul beads to each sample well.
11.1.9. Prepare 4-96 well deep well plates:
11.1.9.1. Tip combs
11.1.9.2. Wash 1: add 0.5 ml 80% ethanol to each well
11.1.9.3. Wash 2: add 1 ml 80% ethanol to each well
11.1.9.4. Elution: add 50 ul water to each well
11.1.10. Place the 96 well deep well plates on the KingFisher Apex as prompted.
11.1.11. Use the RBD protocol (pre-programmed in KingFisher Apex).
11.1.11.1. Binding
11.1.11.1.1. Heat: 65° C.
11.1.11.1.2. Mix: Fast for 5 minutes
11.1.11.1.3. Collect beads: 5 count
11.1.11.2. Wash 1
11.1.11.2.1. Release beads: Fast, 20 seconds
11.1.11.2.2. Mix: Bottom mix, 10 seconds, looping 2 times; medium mix, 10 seconds
11.1.11.2.3. Collect beads: 3 count, 1 second
11.1.11.3. Dry
11.1.11.3.1. 2 minutes, outside well
11.1.11.4. Elution
11.1.11.4.1. Release beads
11.1.11.4.2. Mix: Bottom mix, 15 seconds, looping 6 times; medium mix, 45 seconds
11.1.11.4.3. Collect beads: 8 count, 1 second
11.1.11.5. Final bead
11.1.11.5.1. Collect beads: 10 count, 2 seconds
11.1.11.6. Hold temperature at 4 C
11.1.12. Use 10 ul of sample for PCR (see below).
11.1.13. Freeze remaining eluate at −80° C.
11.2. Set up plate for qPCR
11.2.1. Thaw 1 vial of Master Primer Mix and place on ice. Use a 0.55 ml vial for a full plate and 0.26 ml vial for a half plate.
11.2.2. Vortex well.
11.2.3. Add equal volume of TaqPath to vial of Maser Primer Mix (0.55 ml TaqPath to 0.55 ml Master Primer Mix).
11.2.4. Mix with a P1000. Do not vortex.
11.2.5. Place 10 ul TaqPath/Primer Master Mix into each well for 3 controls and each patient sample.
11.2.6. Add 10 ul positive control (PC) to 1 well containing Master Mix. See Exemplary Sample Set Up.
11.2.7. Add 10 ul extraction control (EC) to 1 well containing Master Mix. See Exemplary Sample Set Up
11.2.8. Add 10 ul nuclease-free water (NC) to 1 well containing Master Mix. See Exemplary Sample Set Up
11.2.9. Add 10 ul of each sample to well containing Master Mix. See Exemplary Sample Set Up.
11.2.10. Seal the plate with Microfilm B.
11.2.11. Spin the plate in refrigerated centrifuge at 3000 RPM for 2 minutes at 4° C.

### Exemplary Sample Set Up:

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | | | | | | |
|---|---|---|---|---|---|---|---|
| A | PC | EC | S1 | S2 | . . . | | |
| B | | | | | | | |
| C | | | | | | | |
| . . . | | | | | | S93 | NC |

NC = no template control,
EC = extraction control,
PC = positive control,
S# = sample

11.3. Run qPCR
11.3.1. Place the plate into the qPCR machine.
11.3.2. Select the protocol “SARS-nCoV-2 test” from the Saved Files library.

| Stage | Temp | Time | | Repeat |
|---|---|---|---|---|
| 1 | 25° C. | 2 | minutes | X1 |
| 2 | 50° C. | 15 | minutes | X1 |
| 3 | 95° C. | 2 | minutes | X1 |
| 4, Step 1 | 95° C. | 3 | seconds | X45 |
| 4, Step 2 | 55° C. | 30 | seconds | |

11.3.3. Name Plate (Plate ID): <date-SARS-CoV-2 initials #>
11.3.4. Review information on screen and on the worksheet with a second technologist and confirm accuracy.
11.3.5. Tap RUN.
11.4. Data Interpretation
11.4.1. Interpretation of controls

| | Expected Control Results | | |
|---|---|---|---|
| Type | N1 primer | N2 primer | RNase P |
| Not template control (NTC) | Neg, ≥40 Ct | Neg, ≥40 Ct | Neg, >40 Ct |
| Positive control (PC) | Pos, <40 Ct | Pos, <40 Ct | Neg, >40 Ct |
| Extraction control (EC) | Neg, ≥40 Ct | Neg, ≥40 Ct | Pos, ≤35 Ct |

11.4.1.1. If NTC is positive for N1, N2, or RP, the PCR reagents may be contaminated. Repeat PCR for entire plate with new PCR reagents.
11.4.1.2. If positive control is negative for N1 or N2 or both, the PCR may have failed. Repeat PCR for the entire plate.
11.4.1.3. If extraction control is positive for N1 or N2 AND there are N1 or N2 positive samples on the plate, the extraction reagents may be contaminated. Repeat extraction and PCR with new extraction reagents for EC and samples with positive results.
11.4.1.4. If extraction control is negative for N1 and N2 and neg or indeterminate for RP AND there are samples with negative or indeterminate RP on the plate, the extraction may have failed. Re-extract and repeat PCR for EC and samples with RP ³35 Ct.
11.4.2. Interpretation of sample results

| N1 | N2 | RNase P | Interpretation | Report |
|---|---|---|---|---|
| Pos (<40 Ct) | Pos (<40 Ct) | Pos (<35 Ct), OR | Detected | Positive |
| | | Ind (35 up to 40 Ct), OR | | |
| | | Neg (≥40 Ct) | | |
| Neg (≥40 Ct) | Neg (≥40 Ct) | Pos (<35 Ct) | Not detected | Not |
| | | | | detected |
| Neg (≥40 Ct) | Neg (≥40 Ct) | Ind (35 up to 40 Ct) | Indeterminate; Repeat | Inconclusive |
| | | | extraction and retest. | |
| | | | If the result is | |
| | | | unchanged, report as | |
| | | | inconclusive and | |
| | | | request another | |
| | | | sample if clinically | |
| | | | indicated | |
| Neg (≥40 Ct) | Neg (≥40 Ct) | Neg, ≥40 Ct | Invalid; | Invalid |
| | | | Repeat extraction | |
| | | | and retest. If the | |
| | | | result is unchanged, | |
| | | | report as invalid and | |
| | | | request another | |
| | | | sample if clinically | |
| | | | indicated | |

| N1 or N2 Pos (<40 Ct), | Pos (<35 Ct), OR | Indeterminate; Repeat | Inconclusive |
|---|---|---|---|
| but not both | Ind (35 up to 40 Ct), | extraction and retest. | |
| | OR Neg (≥40 Ct) | If the result is | |
| | | unchanged, report as | |
| | | inconclusive and | |
| | | request another | |
| | | sample if clinically | |
| | | indicated | |

11.4.2.1. If the controls are expected as above and the sample are both positive for N1 and N2, the test is valid, regardless of whether the sample is positive for RNase P. The sample is positive.
11.4.2.2. If the controls are expected as above and the sample is negative for both N1 and N2, and positive for RNase P, the test is valid and the sample is negative.
11.4.2.3. If the controls are expected as above and the sample is negative for both N1 and N2, and indeterminant for RNase P, the test is inconclusive. Repeat from extraction and qPCR.
11.4.2.4. If the controls are expected as above and the sample is negative for both N1 and N2, but the sample is also negative for RNase P, the test is invalid. Repeat from extraction and qPCR. If the sample remains invalid or is inconclusive, request another sample.
11.4.2.5. If the controls are expected as above and the sample is positive for either N1 or N2 but not both, the test is inconclusive. Repeat from extraction and qPCR.
11.4.2.6. To resolve indeterminate results, extracted RNA may be concentrated by decreasing elution volume at the discretion of the Laboratory Director.
11.5. Repeat testing
11.5.1. If repeating from extraction, re-test as follows:
11.5.1.1. Repeat the extraction.
11.5.1.2. Plate the previously extracted RNA if available in singlicate.
11.5.1.3. Plate the re-extracted RNA in duplicate.
11.5.1.4. Interpret the results using all four sets of data when available or use three when previously extracted RNA is not available.
11.5.2. Use the following sample naming convention:
11.5.2.1. Accession number-R1: PCR with RNA that was previously extracted.
11.5.2.2. Accession number-R2: PCR with RNA that was re-extracted from remaining sample.
11.5.2.3. Accession number-R3: replicate of R2.

12. Limitations
12.1. This test is a laboratory developed test that has been authorized by NYS under an Emergency Use Authorization (EUA) for use by authorized laboratories (fda.gov/emergency-preparedness-and-response/counterterrorism- and emerging-threats/coronavirus-disease-2019-covid-19). This laboratory developed test is not approved by the FDA.
12.2. Performance of this test has only been evaluated in saliva samples placed into GSN buffer.
12.3. Negative results do not preclude SARS-Cov-2 infection.
12.4. Factors such as the optimal sample type or sample collection timing in the course of disease have not been evaluated. Multiple tests from the same patient may be required.
12.5. Mutations within the primer and/or probe binding regions of the viral genome could affect primer binding and therefore may result in failure to detect the presence of virus.
12.6. Self-collection technique or transport conditions may have affected the quality of the sample received.

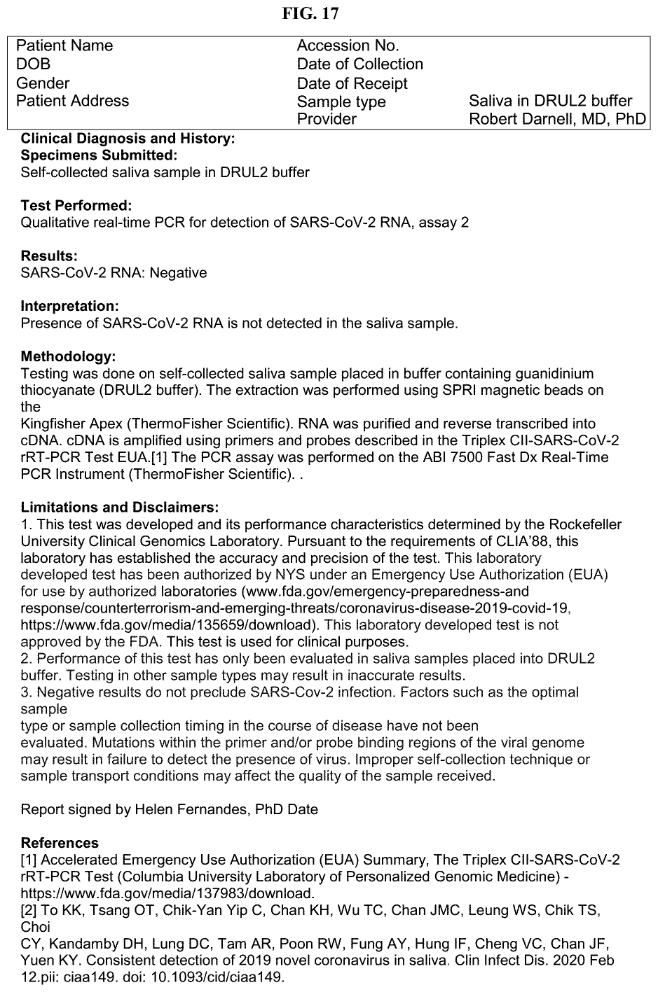

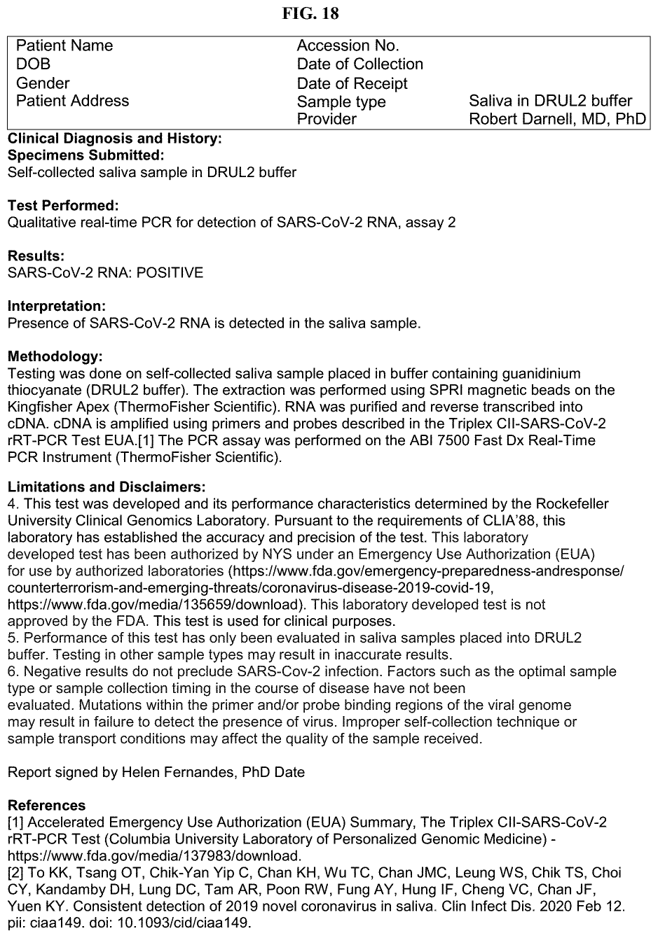

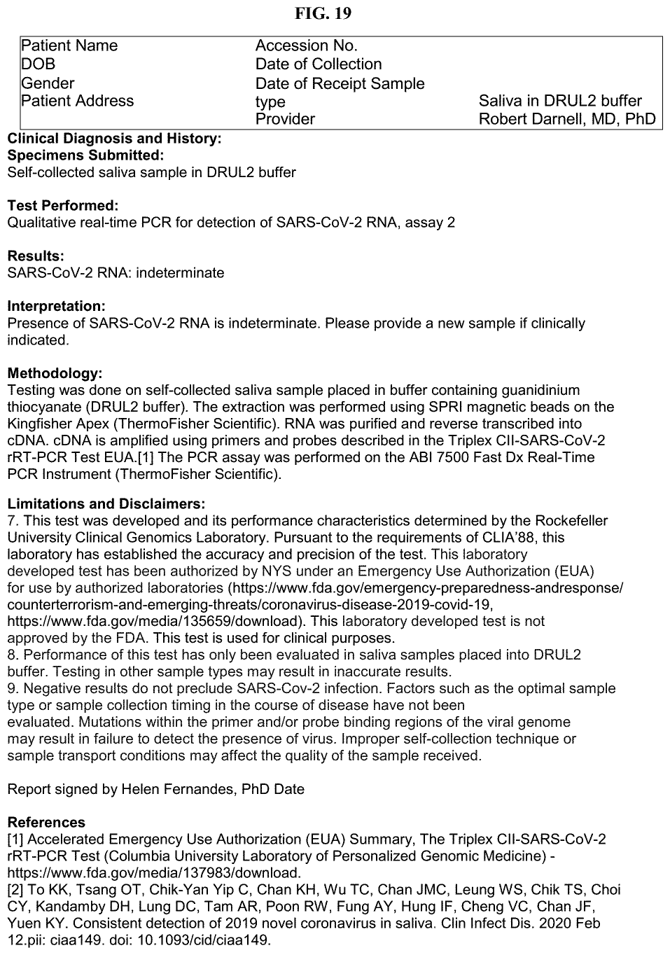

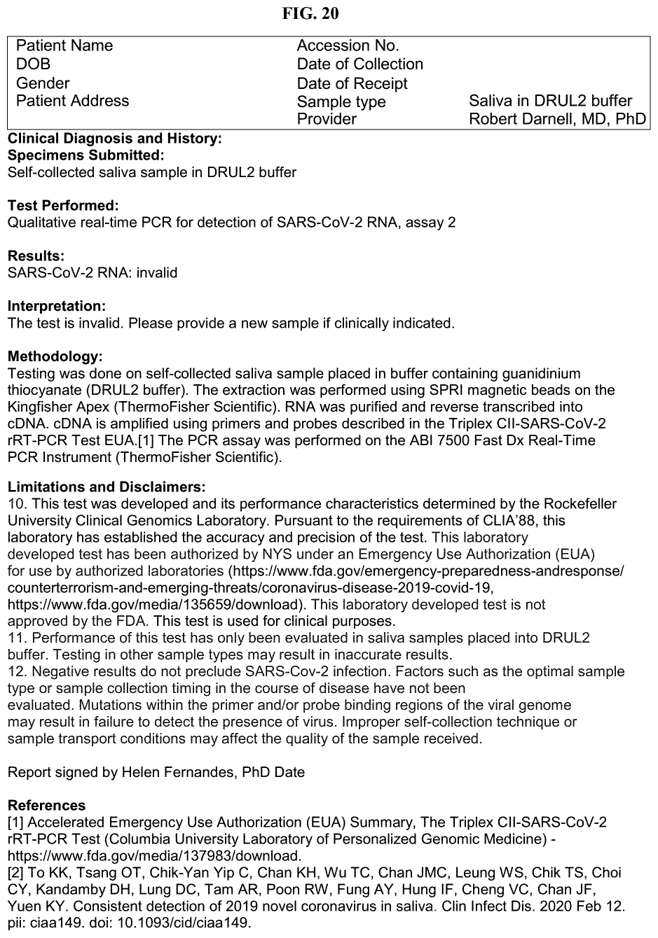

Exemplary Sample Result Reports for test result which is negative, positive, indeterminate and invalid are provided in FIGS. 17-20 respectively.

### Example 7

### Standard Procedure—Proficiency Testing

### Rockefeller University—Clinical Genomics Laboratory

### SOP #14

### Proficiency Testing

### Version 4

Amendments:
V2: Added internal proficiency testing policy and procedures.
V3: Removed internal proficiency testing policy and procedures. We will use CAP PT testing for Sanger sequencing. Corrected numbering under section 3.11
V4: PT for SARS-CoV-2 PCR is added

1. Purpose

This procedure guides the receipt, accessioning, and handling of specimens received at the Rockefeller University Clinical Genomics Laboratory (RUCGL) for proficiency testing (PT).

2. Affected Personnel

Applies to all RUCGL personnel.

3. Policy

The RUCGL participates in external PT programs, when available. If New York State Department of Health (NYSDOH) PT material is not available, the laboratory participates in College of American Pathologists (CAP) surveys. If both are not available for a given assay, the laboratory will complete an internal proficiency test. In accordance with NYSDOH guidelines, the RUCGL enforces the following PT policies:
3.1 PT samples are integrated into the laboratory's routine workflow and tested in the same manner as patient specimens.
3.2 PT samples will be rotated among all technical personnel who routinely perform the testing procedure(s) being evaluated.
3.3 PT samples must be processed by technical personnel who routinely perform the testing procedure(s) being evaluated.
3.4 Repeated testing of PT samples is not allowed unless typically performed on clinical specimens (e.g. instrument failure).
3.5 Inter-laboratory communication or discussion regarding PT samples is not allowed under any circumstances.
3.6 The laboratory will notify NYSDOH within 72 hours of receipt or identification of any NYS proficiency testing samples sent from another laboratory for testing.
3.7 PT samples will not be sent to any other laboratory or location for testing.
3.8 PT attestation will be signed by the Laboratory Director and allanalysts involved.
3.9 The RUGCL will not assign data entry privileges or delegated submitter privileges to any individual who has said privileges at another laboratory.
3.10 PT results must be electronically entered and submitted to CAP from the physical location of the RUCGL.
3.11 PT records are kept in the laboratory for at least 2 years. Records must include, at a minimum, copies of the following:
3.11.1 All sample tracking sheets (library prep sheets, QC output, etc)
3.11.4 Any QC or result reports generated
3.11.5 Attestation form signed by Laboratory Director, submitter, and testing personnel (for alternative assessments, the Clinical Laboratory Internal Proficiency Test Log)
3.11.6 Confirmation of online result submission

4. Proficiency Tests

Currently, no NYSDOH PT testing is available for the tests offered in this laboratory. The laboratory participates in the following CAP surveys twice per year:
4.1 Molecular Genetics (MGL)
4.1.4 Breast Cancer (BRCA½) for BRCA 1/2 Ashkenazi Jewish Founder Mutation Panel
4.1.5 Connexin 26 (GJB2) for Sanger Sequencing
4.1.5.1 Primers are redesigned and documented at each testing timepoint but new primers need not be reordered unless necessary.
4.2 Microsatellite Instability (MSI)
4.3 SARS-COV-2

5. Procedure for Proficiency Testing
5.1 All PT samples received must be tested.
5.2 PT samples are treated as all other clinical samples and must be accessioned.
5.2.4 Last name: CAP PT
5.2.5 First name: CAP specimen ID
5.2.6 Requisitioner: CAP
5.2.7 Collection date: date sample shipped by CAP
5.2.8 Sex: unspecified
5.3 Upon completion of the assay, the results are reported to CAP electronically.
5.4 A hard copy of the Result Form is signed by the Laboratory Director and all technologists involved in assaying the PT samples.

6. Proficiency Test Review of Results
6.1 Review the results within 2 weeks of receipt to confirm that the performance was acceptable.
6.1.4 The Laboratory Director signs the report.
6.2 If the results are not acceptable or indicate shifts, trends, and results that do not meet the RUGCL's performance criteria
6.2.4 Note in Non-conformities Log.
6.2.5 Initiate an investigation of the results to determine the cause of the result and appropriate corrective action, see SOP Complaints, Non-conformities, Investigation and Action.
6.2.6 As part of the investigation, review all test results dating back to the last acceptable PT result, to determine whether the reported results were inaccurate. Notify the requisitioner and issue corrected reports as appropriate.
6.2.7 Document the findings of the investigation, see Investigation Record.
6.2.8 The Laboratory Director documents the review of the investigation and approval of any corrective action taken and forwards to New York State Department of Health.
6.2.9 Successfully complete 2 consecutive PT events.
6.2.10 Retain records for a minimum of 2 years
6.2.11 If NYS DOH mandates or if RUCGL voluntarily chooses cessation of testing of patient samples, samples will be submitted to Memorial Sloan Kettering Cancer Center Laboratories for testing.

### Example 8

### Drul Assays in School Setting: Opening Pre-K with Safe, Simple, Sensitive Saliva Testing for Sars-Cov-2

To address the need for simple, safe, sensitive, and scalable SARS-CoV-2 tests, we validated and implemented a PCR test that uses a saliva collection kit use at home. Individuals self-collected 300 μl saliva in vials containing Darnell Rockefeller University Laboratory (DRUL) buffer and extracted RNA was assayed by RT-PCR (the DRUL saliva assay). The limit of detection was confirmed to be 1 viral copy/μl in 20 of 20 replicate extractions. Viral RNA was stable in DRUL buffer at room temperature up to seven days after sample collection, and safety studies demonstrated that DRUL buffer immediately inactivated virus at concentrations up to 2.75×106 PFU/ml. Results from SARS-CoV-2 positive nasopharyngeal (NP) swab samples collected in viral transport media and assayed with a standard FDA Emergency Use Authorization (EUA) test were highly correlated with samples placed in DRUL buffer. Direct comparison of results from 162 individuals tested by FDA EUA oropharyngeal (OP) or NP swabs with co-collected saliva samples identified four otherwise unidentified positive cases in DRUL buffer. Over six months, we collected 3,724 samples from individuals ranging from 3 months to 92 years of age. This included collecting weekly samples over 10 weeks from teachers, children, and parents from a pre-school program, which allowed its safe reopening while at-risk pods were quarantined. In sum, we validated a simple, sensitive, stable, and safe PCRbased test using a self-collected saliva sample as a valuable tool for clinical diagnosis and screening at workplaces and schools.

### Introduction

The SARS-CoV-2 pandemic has raged in the United States, with over 400,000 deaths by the end of Trump administration [1,2]. Mitigation of this tragedy has struggled alongside the lack of a uniform approach to testing, including mixed messages from the Centers for Disease Control and Prevention (CDC) [3]. These challenges were exacerbated by shortages of testing reagents and supplies [4-6]. Scalable, low cost, accessible testing, in symptomatic and asymptomatic individuals is critical to management of the pandemic. Workplaces and schools need workable strategies to test students, employees and families. Working mothers have been disproportionately affected by the need to care for children who are at home during school closures [7]. Testing is increasingly being used to supplement contact tracing efforts. Collecting, transporting and handling samples in buffer that inactivates virus may decrease exposure risk for healthcare providers and laboratory personnel [11].

Saliva testing is seen as an accessible and scalable means of testing, particularly in the school setting since it does not require technical expertise for collection. However, a wide range of tests have been developed, and those with low sensitivity pose an increased risk of reporting false negatives, which may give a false sense of security and decrease transmission mitigating behaviors. We developed an assay that simplifies sample collection and minimizes contact and exposure, using a kit for self-collection of saliva specimens. The DRUL buffer is a modification of solutions used in RNA extraction that contain guanidinium thiocyanate [12]. Samples were assayed using a test developed using the CDC 2019-nCoV Real-Time PCR Diagnostic Panel primers and probes and RT-PCR [13] as authorized by the NY State Clinical Laboratory Evaluation Program (CLEP) for use as a clinical diagnostic test. Here we report the results of our validation and initial implementation of this testing strategy.

### Materials and Methods

Study subjects: Individuals voluntarily participated in sample collection for serial screening. They were provided with a sample collection kit and instructions (FIG. 1). Protocols for the collection of saliva samples were either approved by the Rockefeller University (RU) Institutional Review Board (IRB) and Biomedical Research Alliance of New York IRB or were deemed not to be human subjects research by the RU IRB. Where required, written or verbal consent was obtained from all volunteers.

Specimen collection and processing: Individuals were instructed to avoid eating or using oral cleansing agents for 30 minutes prior to collection of saliva (or their children's saliva) in a medicine cup, and then transfer 300 μl of saliva using a pre-calibrated plastic bulb into a vial containing 1200 ul of DRUL buffer (Table 43). Samples were stored and transported at room temperature.

TABLE 43
DRUL buffer reagents

| Reagent | Amount needed | | Purpose | Manufacturer | Catalog # |
|---|---|---|---|---|---|
| 5M Guanidine | 59.08 | g | Protein denaturing agent, | Fisher | BP221-1 |
| Thiocyanate | | | Isolation of RNA | BioReagents | |
| 0.5% Sarkosyl | 5 ml of 10% Sarkosyl | | Cell lysis, detergent | Fisher | BP234-500 |
| | | | | BioReagents | |
| 25 mM Sodium | 0.83 | ml | Precipitation of RNA | Invitrogen | AM9740 |

| | | | | |
|---|---|---|---|---|
| Acetate (3M), | | | | |
| pH 5.5 | | | | |
| Nuclease- | Bring up to 100 ml | Dilution | Ambion | AM9932 |
| free Water | | | | |

SARS-CoV-2 assay: In early experiments, RNA was extracted using a modified phenol-chloroform extraction method. 80 μl of 3M sodium acetate, pH 5.5 was added to 800 μl of sample plus buffer and mixed. Then 800 μl of acid-phenol:chloroform pH 4.5 (with IAA, 125:24:1, Ambion, Cat #9720) was added and mixed. Samples were centrifuged at 12,000×g for 10 minutes at 4° C. after which the aqueous phase (750 μl) was placed into a new tube. 750 μl of OmiPur chloroform: Iso-Amyl Alcohol (Calbiochem, Cat #3155) was added, mixed, then centrifuged at 12,000×g for 10 minutes at 4° C. The aqueous phase (550 μl) was placed into a new tube to which 2 μl GlycoBlue (Invitrogen, Cat #AM9515) was added and mixed. 550 μl of ice cold 100% isopropanol was then added and incubated for 15 minutes at −80° C. or overnight at −20° C. Samples were centrifuged at 20,000×g for 20 minutes at 4° C. and supernatant removed without disturbing the pellet. 1 ml of cold 75% ethanol was added to the pellet and centrifuged at 20,000×g for 5 minutes at 4° C. The supernatant was removed and the pellet dried at room temperature for 10 minutes and resuspended in 35 μl of nuclease-free water. In later experiments, RNA was extracted using a column extraction method with a commercial kit (Qiagen, QIAamp DSP Viral RNA Mini Kit, Cat #61904) according to the manufacturer's instructions. RNA was eluted in 35 μl of nuclease free water.

cDNA was amplified using TaqPath 1 Step RT-PCR (Life Tech, Cat #A15300) with CDC validated primers and probes (IDT, CDC Emergency Use Authorization Kit) using the Bio-Rad CFX96 C1000 Touch Real-Time PCR Detection System. Samples were considered interpretable if the housekeeping control (RNase P) cycle threshold (Ct) was less than 40 and viral RNA was considered detected with both viral primers/probes (N1 and N2) at Ct<40.

To determine the limit of detection (LOD) of the DRUL saliva assay, contrived clinical specimens (found to be viral-free in the absence of synthetic RNA) were made by spiking in known amounts of quantitative synthetic RNA from SARS-Related Coronavirus 2 (BEI Resources, Cat #NR-52358) into 300 μl of saliva added to indicated amounts of DRUL buffer. Saliva collected from normal volunteers previously determined to be negative for SARS-CoV-2 was pooled and spiked with DRUL buffer containing synthetic SARS-CoV-2 RNA (BEI Resources, Cat #52358).

To assess sensitivity and specificity of the DRUL saliva assay, thirty NP swab samples were obtained from New York City Public Health Laboratory (NYC PHL). The NP swabs were collected using standard methods by a provider and placed in 3 ml of VTM, and 300 μl of the VTM was added to 1200 μl DRUL buffer at NYC PHL and then transported to the Darnell laboratory for testing.

To determine the ability of DRUL buffer to inactivate virus, Huh-7.5 cells were plated at 1.67×105 per well in each well of 6 well plates and allowed to adhere overnight. Human coronavirus 229E (3.66×106 PFU/ml) was used as a surrogate for SARS-CoV-2. Mixtures of DRUL buffer and virus at volume ratios of 1:2, 1:3, 1:4, 1:5, 1:6 and 1:10 were incubated overnight and added to the Huh-7.5 cells the following morning. The viability of the Huh-7.5 cells was assessed after 3 and 5 days of incubation, yielding approximate TCID50 values. The TCID50 was calculated as the concentration of virus that when diluted in a defined concentration of DRUL buffer led to 50% viability of Huh-7.5 cells on day 3 post inoculation.

To measure viral RNA stability in DRUL buffer, specified concentrations of human coronavirus 229E were incubated with saliva and DRUL buffer and assayed for presence of viral RNA after overnight incubation or after seven days at room temperature and at 0° C., 25° C., and 38° C. for seven days. cDNA was amplified using iScript Reverse Transcription Supermix (BioRad, Cat #1708841) and two primer sets for human coronavirus 229E, set 1: forward-TGAAGATGCTTGTACTGTGGCT (SEQ ID NO:22) and reverse-CTGTCATGTTGCTCATGGGG (SEQ ID NO:23), set 2 forward-AGATGCTTGTACTGTGGCTTCT (SEQ ID NO:24) and reverse-GTCATGTTGCTCATGGGGGAG (SEQ ID NO:25) (IDT, custom) from 5′ to 3′ [8] and FASTSTART Universal SYBR Green Master Mix (Millipore Sigma, Cat #4913914001) using the Bio-Rad CFX96 C1000 Touch Real-Time PCR Detection System. Samples were considered interpretable if the house keeping control (beta-actin) Ct was <40 and viral RNA was considered detected if Ct for both viral primers were <40.

### Results

DRUL Saliva Assay Validation

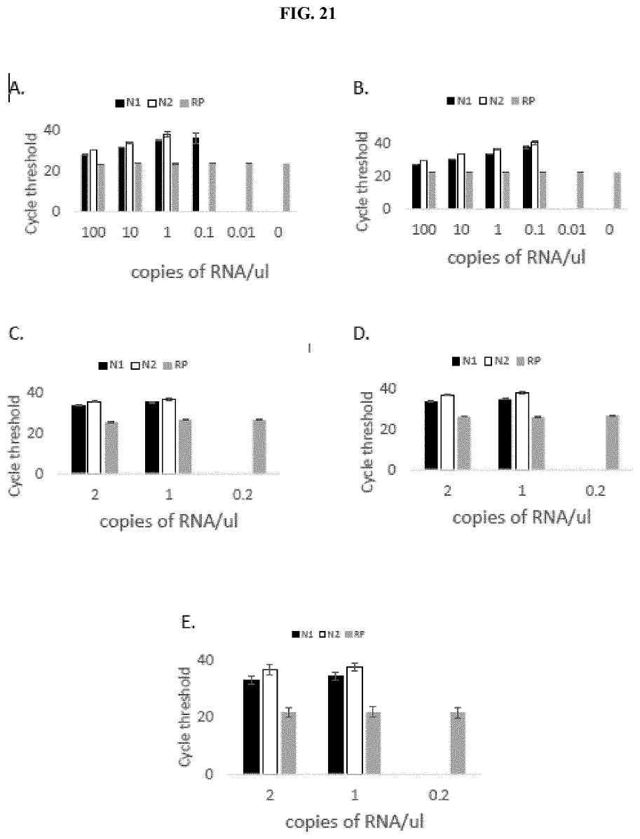

To establish the LOD of the DRUL saliva assay, simulated specimen matrix was made using 5 pooled saliva samples and DRUL buffer spiked with 10-fold serial dilutions of synthetic SARS-CoV-2 RNA. Samples were extracted using a phenol-chloroform or column-based method. Dilutions were tested in triplicate at each concentration of viral RNA. The LOD was determined to be 1 copy/μ1 with both extraction methods (FIGS. 21, A and B). The LOD was confirmed with 20 replicates, each spiked with 2, 1, and 0.2 copies/μ1 of synthetic RNA, using both extraction methods (FIGS. 21, C and D) and with 20 separate extractions, each spiked with 2, 1, and 0.2 copies/μ1 of synthetic RNA using the column-based extraction method (FIG. 21E).

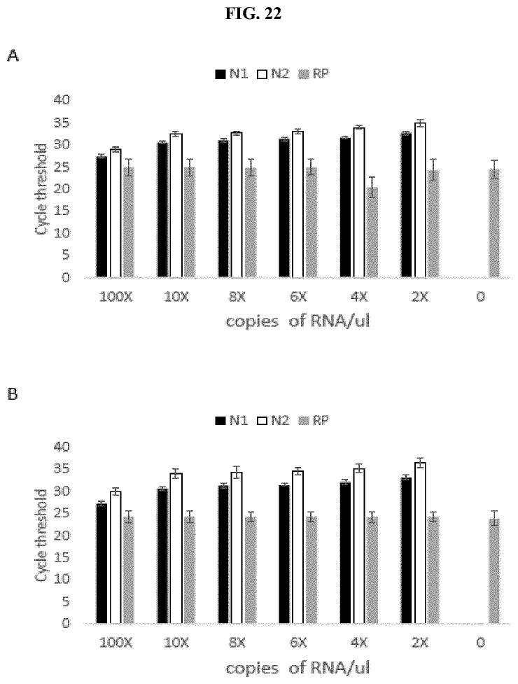

Given a paucity of positive samples to use for a clinical evaluation study, we created simulated positive samples representing various viral concentrations. Synthetic viral RNA was spiked into five individual specimen matrices at 2, 4, 6, 8, 10, and 100 times the confirmed LOD (1 copy/μ1) to simulate a range of viral load. RNA was extracted using the phenol or column-based method. In addition, 10 negative specimen matrices were assayed. As expected, mean Cts decreased with increasing viral RNA concentrations using both extraction methods (FIGS. 22, A and B). RNase P Cts was <40 and Cts were similar in all samples.

To compare the DRUL saliva assay with a clinically validated platform, we obtained 30 NP swab samples that had tested positive with a wide range of Cts (17.3 to 39.5 on the N2 target) on the Cepheid Xpert Xpress SARS-CoV-2 assay [14]. We detected 30/30 positives (100% sensitivity), and comparison of the Ct values of the N2 target on both platforms revealed that they were highly correlated (FIG. 5, Pearson correlation, R2=0.96). These results indicate that the DRUL saliva assay performed with high specificity, and with quantitative results that were concordant over a wide range (4.8×10⁶-fold) of detectable viral RNA in clinical samples, as confirmed by comparison to the Cepheid Xpert Xpress SARS-CoV-2 assay.

To further investigate the sensitivity and specificity of the DRUL saliva assay, we compared 63 OP swab results collected from RU Occupational Health Services, which were tested at commercial laboratory and with co-collected self-collected saliva samples in DRUL buffer. The majority of co-collected specimens (57/63, 90.5%) were negative by both assays (Table 44). Of the remaining six specimens, SARS-CoV-2 RNA was detected in the saliva specimen in three participants. Of these three, two of the co-collected OP specimens were negative and one was indeterminate by the Commercial Laboratory A test. These individuals were symptomatic. Three additional participants were negative by OP swab, and the saliva test was invalid. RNaseP target was not detected, which we most commonly found correlated with insufficient saliva specimen, although in some cases inhibitors may have been present.

TABLE 44
Comparison of SARS-CoV-2 DRUL assay
with commercially tested OP swabs

| | | Darnell Lab-Saliva | | |
|---|---|---|---|---|
| | | Positive | Negative | Invalid |
| Commercial Lab | Positive | 0 | 0 | 0 |
| A- OP swab | Negative | 2 | 57 | 3 |
| | Inconclusive | 1 | 0 | 0 |

In a second study to assess sensitivity and specificity, we compared 99 NP swab samples collected by healthcare providers at New York City Health and Hospital—Elmhurst (tested at Commercial Laboratory B) with co-collected, self-collected saliva samples in DRUL buffer (Table 45). All samples but one were negative by both assays. The DRUL saliva assay identified one positive sample of the 99 which was negative by NP swab in an asymptomatic individual. At the time these experiments were done, the turnaround time for results from paired samples in Commercial Laboratory B was three to five days, while results from the DRUL saliva assay were generally available the next day, including the one positive sample. These studies taken together suggest similar, if not higher sensitivity of the DRUL saliva assay than commonly accepted viral assays using OP or NP swabs.

TABLE 45
Comparison of SARS-CoV-2 DRUL assay
with commercially tested NP swabs

Darnell Lab-Saliva

| | | Positive | Negative |
|---|---|---|---|
| Commercial Lab B- | Positive | 0 | 0 |
| NP swab | Negative | 1 | 98 |

To assess the stability of viral RNA in DRUL buffer, we titrated concentrations of human coronavirus 229E into saliva and DRUL buffer and compared Ct values of samples incubated overnight or after 7 days at room temperature. There was no significant difference between the Ct values of samples incubated overnight and those incubated for 7 days (FIGS. 10A and B). We further assessed the stability of viral RNA incubated at 0° C., 25° C., and 38° C. for 7 days, to mimic potential temperature ranges during sample transport and did not find a significant difference in Ct values (FIG. 10C).

To evaluate the effect of DRUL buffer on viral infectivity, we used human coronavirus 229E as a surrogate for SARS-CoV-2. We assessed the viability of Huh-7.5 cells, a well characterized, adult hepatocellular carcinoma cell line, after exposure to various dilutions of coronavirus (stock 3.66×10⁶ PFU/ml) in DRUL buffer. Huh-7.5 cell survival indicates that the virus was inactivated by the DRUL buffer, and cells remained viable after exposure of stock virus diluted with DRUL buffer at ratios of 1:4 (DRUL:virus), indicating that DRUL buffer completely inactivates virus at 2.75×106 PFU/ml (FIGS. 11A and 11B). At DRUL to virus ratio of 1:5 (3.41×106 PFU/ml), approximately half the Huh-7.5 cells were lysed at day 5 indicating viral survival, and this value was taken as a conservative estimate of the TCID50. We compared this with the AVL buffer, a component of the QIAamp Viral RNA kit that the CDC determined to inactivate virus [7]. AVL buffer inactivated virus at a buffer to virus ratio of 1:3 (2.44×106 PFU/ml) but not at 1:4, indicating that DRUL buffer inactivates virus at a level comparable to AVL buffer (FIGS. 11A and B). In control samples, cell lysis occurred when we exposed Huh-7.5 cells to as little as 4 PFU/ml human coronavirus E229 without DRUL buffer, and no cell lysis occurred when DRUL buffer was added without virus.

To determine the minimum incubation time required for the DRUL buffer to inactivate virus, we incubated DRUL buffer and virus at a ratio of 1:4 for 60 minutes, 10 minutes and 10 seconds before incubating with Huh-7.5 cells. We found that 100% of the Huh-7.5 cells were viable at 3 and 5 days after incubation with virus exposed to DRUL buffer for as little as 10 seconds (FIG. 4C). Taken together these results indicate that DRUL buffer nearly instantly inactivates live, high titer coronavirus.

Clinical Use of the DRUL Saliva Assay

These validation data were submitted to New York State CLEP and the DRUL saliva assay was subsequently authorized for use as a clinical diagnostic test and are currently pending final approval. The assay was used in 3,724 samples between May and October of 2020 from individuals who ranged in age from 3 months to 92 years. We began with testing symptomatic employees and asymptomatic essential employees coming onto the RU campus.

In July of 2020, the RU Child and Family Center (CFC) for children of employees between the ages of three months and five years reopened on a pilot basis, enrolling 58 children in July and August of 2020, then 87 children starting in September. Each child, teacher and staff member was tested weekly, and parents were also offered testing. 2117 kits were distributed over 12 weeks, which were typically taken home, where saliva was collected and added to DRUL buffer with a plastic bulb syringe. Electronic sample submission forms linked to a personalized registration data were completed for each sample, and tubes returned to RU the following day.

Over these 12 weeks, only one asymptomatic parent tested positive. The parent was isolated, the child (a contact) was quarantined, and the classroom closed. Overall, 26 children school days were missed (number of children in room×number of days classroom closed or school days missed; Table 46). All other tests among the children, teachers, staff, and parents were negative, allowing these rooms to remain open, consistent with (or more conservative than) NYS/NYC DOE school guidance. There were three additional room closures due to symptomatic (as defined by CDC guidelines) children or teachers who tested negative, resulting in 46 children school days missed. There were 72 missed children school days out of 4205 (1.7%) over the course of 12 weeks.

TABLE 46
Child and Family Center Closures

| | | Total days | School | Children |
|---|---|---|---|---|
| | | closed | days closed | school days |
| | | (no.) | (no.) | missed (no.) |
| 1 | symptomatic child | 6 | 4 | 28 |
| 2 | positive parent | 6 | 4 | 26* |
| 3 | symptomatic teacher | 3 | 1 | 10 |
| 4 | symptomatic child | 4 | 2 | 8 |

### Discussion

Here, we report the validation of the DRUL saliva assay for SARS CoV-2 molecular testing as performed at RU. This assay was easy to administer, using a self-collection kit that could be performed at home by adults or by older children under adult supervision. RTPCR assays, using either traditional phenol-chloroform or column-based extraction methods revealed that the assay was extremely sensitive, with a LOD of 1 copy/μl of viral RNA, and was found to perform nearly identically to a clinical platform (Cepheid Xpert Xpress SARS-CoV-2 assay). Moreover, the assay was found to be at least as sensitive as OP and NP swabs assessed by commercial laboratories using FDA approved molecular tests.

The DRUL saliva assay was developed with the goal of overcoming the early obstacles to widespread SARS-CoV-2 testing, such as shortages of reagents and specialized supplies, healthcare provider access, and PPE for healthcare providers. This method also limits potential exposure during transit and of laboratory personnel during performance of the assay. The TCID50 of virus (˜2.64×106 PFU/ml) diluted 1:4 (v:v) in DRUL buffer was found to compare favorably to commercial inactivation buffers (Qiagen's AVL buffer, FIG. 11 or the SDNA-1000 saliva collection device with an estimated TCID50 of ˜1×104 PFU/ml used by Rutgers Clinical Genomics Laboratory TaqPath SARS-CoV-2 Assay) [13,15].

Such solutions have a health hazard label that grades them as less toxic than household bleach. DRUL buffer kits are distributed with appropriate cautions and instructions on what to do in case of a spill or contact. To further minimize risk, we have recently succeeded in decreasing the required volume of DRUL from 1200 μl to 300 μl with similar results (see above Examples).

The DRUL saliva assay was used for testing symptomatic individuals and screening asymptomatic essential employees on the RU campus over the course of 6 months. It was easy to use across a variety of ages and individuals with varied backgrounds. During this time the assay was used to aid in the reopening of a childcare center that enrolled children as young as three months old. The use of the test minimized the number of days a classroom closed and allowed the rest of the center to remain open safely. With testing, 98.7% student attendance was possible, along with reassurance that both they and their teachers had undetectable viral RNA on a weekly basis. As SARS-CoV-2 infection remain a significant clinical issue, the DRUL saliva test offers a simple, safe, and cost-effective method for use as part of highly scalable “back to work/school” strategies.

### REFERENCES

1. CDC. COVID-19 Cases, Deaths, and Trends in the US. 28 Mar. 2020 [cited 22 Nov. 2020]. Available: https://covid.cdc.gov/covid-data-tracker
2. Institute for Health Metrics and Evaluation. COVID-19 Projections. [cited Nov. 22, 2020]. Available: https://covid19.healthdata.org/
3. CDC. Overview of testing for SARS-CoV-2 (COVID-19). 10 Dec. 2020 [cited 18 Feb. 2021]. Available: https://www.cdc.gov/coronavirus/2019-ncov/hcp/testingoverview. html
4. Akst J. RNA Extraction Kits for COVID-19 Tests Are in Short Supply in US. 11 Mar. 2020 [cited 22 Nov. 2020]. Available: https://www.the-scientist.com/newsopinion/rna-extraction-kits-for-covid-19-tests-are-in-short-supply-in-us-67250
5. Esbin M N, Whitney O N, Chong S, Maurer A, Darzacq X, Tjian R. Overcoming the bottleneck to widespread testing: a rapid review of nucleic acid testing approaches for COVID-19 detection. RNA. 2020; 26: 771-783.
6. Baird R P. Why Widespread Coronavirus Testing Isn't Coming Anytime Soon. The New Yorker. 24 Mar. 2020.
7. Bateman N R M. Why has COVID-19 been especially harmful for working women? In: Brookings [Internet]. 14 Oct. 2020 [cited 22 Nov. 2020]. Available: https://www.brookings.edu/essay/why-has-covid-19-been-especially-harmful-forworking-women/
8. CDC. Information for Laboratories about Coronavirus (COVID-19). 17 Nov. 2020 [cited 18 Feb. 2021]. Available: https://www.cdc.gov/coronavirus/2019-ncov/lab/index.html
9. Callahan C, Lee R, Lee G, Zulauf K E, Kirby J E, Arnaout R. Nasal-Swab Testing Misses Patients with Low SARS-CoV-2 Viral Loads. medRxiv. 2020. doi:10.1101/2020.06.12.20128736
10. Basu A, Zinger T, Inglima K, Woo K-M, Atie O, Yurasits L, et al. Performance of Abbott ID Now COVID-19 Rapid Nucleic Acid Amplification Test Using Nasopharyngeal Swabs Transported in Viral Transport Media and Dry Nasal Swabs in a New York City Academic Institution. J Clin Microbiol. 2020; 58. doi:10.1128/JCM.01136-20
11. Kaiser J. The catalyst. Science. 2014. pp. 1112-1115. doi:10.1126/science.345.6201.1112
12. Chomczynski P, Sacchi N. Single-step method of RNA isolation by acid guanidinium thiocyanate-phenol-chloroform extraction. Anal Biochem. 1987; 162: 156-159.
13. CDC. CDC 2019-Novel Coronavirus (2019-nCov) Real-Time RT-PCR Diagnostic Panel. In: In Vitro Diagnostics EUAs [Internet]. Feb. 4, 2020 [cited Feb. 10, 2021]. Available: https://www.fda.gov/medical-devices/coronavirus-disease-2019-covid-19-emergency-use-authorizations-medical-devices/vitro-diagnostics-euas
14. Cepheid. Xpert Xpress SARS-CoV-2 Instructions for Use For Use Under an Emergency Use Authorization Only. 7 Jan. 2021 [cited 18 Feb. 2021]. Available: https://www.fda.gov/media/136314/download
15. Spectrum Solutions. Emergency Use Authorization (EUA) Summary SDNA-1000 Saliva Collection Device. [cited 2 Mar. 2021]. Available: https://www.fda.gov/media/142907/download #: text=The %20SDNA %2D1000%20 Saliva %20Collection %20Device %20consists%20of%20a%20plastic,containing%20 Spectrum's%20patented%20stabilizing% 20solution.

### Example 9

### Sars-Cov-2 Variants and Vaccine-Breakthrough Infections

Emerging SARS-CoV-2 variants are of clinical concern. Among a cohort of 417 individuals who were at least two weeks beyond Pfizer or Moderna vaccinations, we identified two with “vaccine breakthrough.” Each developed COVID-19 symptoms and tested positive for SARS-CoV-2 by PCR, despite evidence of vaccine efficacy. Viral sequencing revealed variants of likely clinical importance, including E484K in one patient and three mutations (T95I, del142-144, D614G) found in both. These observations indicate a potential risk of illness after successful vaccination and subsequent infection with variant virus and support continued efforts to prevent and diagnose infection and to characterize variants in vaccinated individuals.

### Introduction

SARS-CoV-2 infections caused over 83 million known cases of COVID-19 by the end of 2020, but tremendous progress has been made with vaccine and antibody therapies being authorized and deployed. These strategies are directed at the viral spike protein, but the emergence of viral variants, particularly in the S-gene, threatens their continued efficacy.

These concerns have provided an impetus to increase testing and sequencing of positive cases to understand the transmissibility, virulence, and the ability of variants to evade current vaccines. Indeed, New York City has seen a concerning rise of viral variants, which now account for more than 72% of new cases as of Mar. 30, 2021, with the vast majority being variants that first emerged in the UK (B.1.1.7, 26.2%) and New York (B.1.526, 42.9%) [1]. Two levels of concern relate to the ability of variants to evade vaccine-induced immunity—their ability to cause asymptomatic infection, and thereby promote viral spread, and their ability to cause morbidity. Both are important, both need to be considered independently, and both are largely unknown.

We describe two cases of previously fully vaccinated individuals who developed breakthrough infections with SARS-CoV-2 variants harboring a number of substitutions of interest. Despite evidence that the initial vaccination led to a robust antibody response to spike protein in Patient 1, saliva-based PCR screening at the Rockefeller University identified infection 19 days after booster vaccination. Testing identified a second positive case 37 days after completing vaccination. Together these observations provide support for current strategies to monitor multiple variables proactively—viral testing of symptomatic and asymptomatic individuals, sequencing of viral RNA, and monitoring neutralizing antibody titers, particularly in positive individuals who have been vaccinated.

### Methods

Specimen Collection and Processing

Beginning fall of 2020, all employees students coming to the Rockefeller University campus (˜1400 per week) have been tested at least weekly by a saliva-based PCR test developed in the Darnell CLIA-CLEP lab (PFI-9216) and approved for clinical use by a New York State EUA. Following NY state regulations regarding eligibility, 417 employees who were at least two weeks beyond a second dose of either the Pfizer/BioNTech or Moderna vaccine were tested between 1/21/2021 and 3/17/2021 and have continued weekly testing. The demographic characteristics of these 417 individuals and 1,491 unvaccinated individuals tested in parallel by Rockefeller University during this time are shown in Table 47.

TABLE 47
Demographics on vaccinated and unvaccinated cohorts tested at the Rockefeller
University between Jan. 21, 2021 and Mar. 17, 2021. Vaccinated refers
to participants who were at least 2 weeks beyond the second dose of
either Pfizer/BioNTech or Moderna vaccines by Mar. 17, 2021.

| | Vaccinated Cohort (n = 417) | | Unvaccinated Cohort (n = 1491) | |
|---|---|---|---|---|
| Gender | | | | |
| Female | 186 | 44.60% | 698 | 46.81% |
| Male | 228 | 54.20% | 780 | 52.31% |
| Other | 58 | 1.20% | 13 | 0.87% |
| Average age (years) | 47.2 | | 34.9 | |
| Ethnicity | | | | |
| Not hispanic | 324 | 77.70% | 299 | 20.05% |
| Hispanic | 65 | 15.59% | 1063 | 71.29% |
| Not reported | 28 | 6.71% | 129 | 8.65% |
| Race | | | | |
| White | 253 | 60.67% | 708 | 47.48% |
| Black | 36 | 8.63% | 153 | 10.26% |
| Asian | 58 | 14.15% | 332 | 22.27% |
| More than one | 22 | 5.28% | 88 | 5.90% |
| Unknown/unreported | 45 | 10.79% | 203 | 13.62% |
| Native American | 2 | 0.48% | 4 | 0.27% |
| Pacific Islander | 0 | 0.00% | 3 | 0.20% |
| Education | | | | |
| College Grad | 346 | 82.97% | 1059 | 71.03% |
| Some College | 27 | 6.47% | 131 | 8.79% |
| High School Graduate | 36 | 8.63% | 149 | 9.99% |
| Some High School | 5 | 1.20% | 27 | 1.81% |
| Under 18 years old | 0 | 0.00% | 125 | 8.38% |
| Unreported | 3 | 0.72% | 0 | 0.00% |
| Health Care worker | | | | |
| No | 352 | 84.41% | 1484 | 99.53% |
| Yes | 65 | 15.59% | 7 | 0.47% |
| First responder | | | | |
| No | 406 | 97.36% | 1480 | 99.26% |
| Yes | 11 | 2.64% | 11 | 0.74% |
| Use public | | | | |
| Transportation | | | | |
| No | 336 | 80.58% | 1139 | 76.39% |
| Yes | 83 | 19.42% | 352 | 23.61% |
| Net PCR Test Results | | | | |
| Positive | 2 | 0.48% | 32 | 2.15% |

Individuals were instructed to collect their saliva in a medicine cup and transfer 300 μl into a vial containing 300 ul of DRUL buffer (5 M guanidine thiocyanate, 0.5% sarkosyl, and 300 mM sodium acetate, pH 5.5) [2]. Samples were processed on Thermo Kingfisher Apex for rapid RNA purification, and cDNA was amplified using TaqPath 1 Step RT-PCR (Life Tech, Cat #A15300) and FDA-EUA validated multiplexed primers and probes (Table 48) using the ABI 7500Fast Dx PCR Detection System. Samples were considered interpretable if the housekeeping control (RNase P) cycle threshold (Ct) was less than 40 and viral RNA was considered detected with both viral primers/probes (N1 and N2) at Ct<40.

TABLE 48

| Custom Primer/Probes*** | Sequence | Storage |
|---|---|---|
| SARS-COV-2 forward primer for N1 target | GACCAGGAACTAATCAGACAAGG | Room |
| (nCoV_NF1) | (SEQ ID NO: 26) | temp |
| SARS-COV-2 reverse primer for N1 target | TCAACCACGTTCCCGAAGG | Room |
| (nCoV_NR1) | (SEQ ID NO: 27) | Temp |
| SARS-Cov-2 FAM Probe for N1 target | CGACATTCCGAAGAACGCTGAAGCG | Room |
| (nCoV_NP1) | (SEQ ID NO: 28) | temp |
| SARS-COV-2 forward primer for N2 target | GCATCCAAATTGGATGACAAAGATC | Room |
| (nCoV_NF2) | (SEQ ID NO: 29) | Temp |
| SARS-COV-2 reverse primer for N2 target | TAGGCTCTGTTGGTGGGAATG | Room |
| (nCoV_NR2) | (SEQ ID NO: 30) | Temp |
| SARS-COV-2 Quasar 670 Probe for N2 target | CATTTTGCTGAATAAGCATATTGACGC | Room |
| (nCoV_NP2) | (SEQ ID NO: 31) | temp |
| RNAse P forward primer (RP_F) | AGATTTGGACCTGCGAGCG | Room |
| | (SEQ ID NO: 32) | temp |
| RNAse P reverse primer (RP_R) | GAGCGGCTGTCTCCACAAGT | Room |
| | (SEQ ID NO: 33) | Temp |
| RNAse P CAL Fluor Orange 560 probe (RP_P) | TTCTGACCTGAAGGCTCTGCGCG | Room |
| | (SEQ ID NO: 34) | temp |

***Probes are all manufactured by LGC Bioresearch Tecnologies

Viral Load Calculation

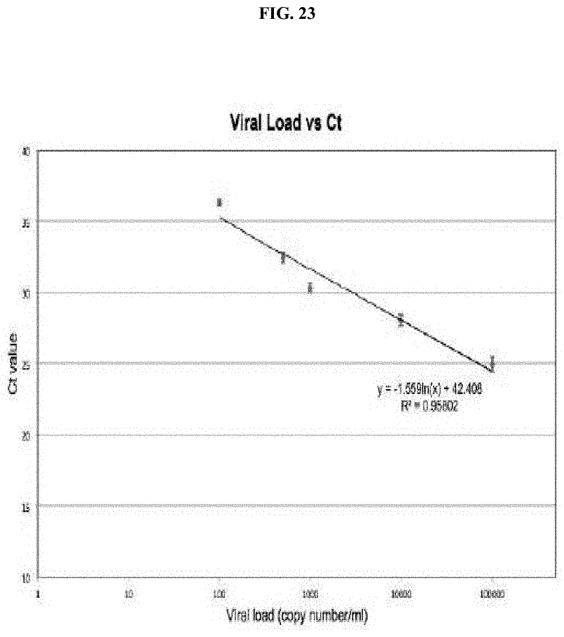

Viral load per ml of saliva was calculated using chemically inactivated SARS-CoV-2 virus (Zeptometrix) spiked into saliva at various dilutions. Extractions and RT-PCR were performed as described previously to determine the corresponding Ct values for each dilution (FIG. 23).

Targeted Sequencing

Patient RNA samples were reverse transcribed using the iScript mix (Bio-Rad, #1708890) according to the manufacturer's instructions. cDNA was PCR amplified using 2 primer sets (F1: CCAGATGATTTTACAGGCTGC (SEQ ID NO:35) and R1: CTACTGATGTCTTGGTCATAGAC (SEQ ID NO:36); F2:CTTGTTTTATTGCCACTAGTC (SEQ ID NO:37) and R1). PCR products were then gel extracted and sent to Genewiz for Sanger Sequencing.

Neutralization Assay

Neutralization assays using SARS-CoV-2 spike pseudotyped replication defective HIV-1 were performed as previously described [3]. NT50 was calculated as an average of 3 independent experiments, each performed using technical duplicates, and statistical significance determined using the two-tailed Mann-Whitney U test.

Whole Viral RNA Genome Sequencing

Total RNA was extracted as described above, and a meta-transcriptomic library was constructed for paired end (150-bp reads) sequencing using an Illumina MiSeq. Libraries were prepared using the SureSelect XT HS2 Target Enrichment kit (Agilent Technologies, #5191-6688) and SSEL CD PanHumanCoronavirus panel (Agilent Technolgies, #5191-6838) according to the manufacturer's instructions. FASTQ files were trimmed using the AGeNT-2 software (version 2.0.5) and used for downstream analysis. SARS-CoV-2 genome was assembled using MEGAHIT with default parameters, and the longest sequence (30,005 nt) was subjected to Nextclade (clades.nextstrain.org) to assign the Glade and call mutations. Detected mutations were confirmed by aligning RNAseq reads on SARS-CoV-2 reference genome sequence (NC_045512) with Burrows-Wheeler Aligner (BWA).

Patient Histories

Patient 1 is a healthy 51-year-old female with no COVID-19-related comorbidities who received two doses of Moderna mRNA-1273 COVID-19 vaccine on 1/21/2021 and 2/19/2021 who adhered strictly to routine precautions. Ten hours following the second vaccine dose she developed “flu-like” muscle aches that resolved the following day. On 3/10/2021, 19 days after completing vaccination, she developed a sore throat, congestion and headache, and tested positive for SARS-CoV-2 RNA at Rockefeller University later that day. On 3/11/2021 she lost her sense of smell. Her symptoms gradual resolved over a week.

Patient 2 is a healthy 65-year-old female with no COVID-19-related comorbidities who received two doses of Pfizer-BioNTech BNT162b2 COVID-19 vaccine on 1/19/2021 and 2/9/2021 and developed pain in the inoculated arm for the following two days. On 3/3/21 her unvaccinated partner tested positive for SARS-CoV-2, and on 3/16/21 she developed fatigue, sinus congestion and a headache. On 3/17/21 she felt worse and tested positive for SARS-CoV-2 RNA, 37 days after completing vaccination. Her symptoms plateaued and began to resolve on 3/20/21.

### Results

Serial saliva-PCR tests were performed before and during the course of the patients' illness (Table 49). At the time of diagnosis, PCR testing of Patient 1 gave a Ct value that corresponded to a viral load of ˜195,000 copies/ml of saliva and Patient 2-400 copies/ml (FIG. 23). Given the unusual clinical histories, RNA obtained from the saliva samples was sequenced following reverse transcription and targeted PCR amplification of the SARS-CoV-2 S gene. This revealed a number of sequence differences compared to the original Wuhan sequence, including several that have been suggested to be of potential clinical concern. In Patient 1, these included identification of the E484K, which confers resistance to a commonly elicited class of neutralizing antibodies [4,5] and D614G, and in Patient 2, D614G (Table 50).

TABLE 49
Viral Loads in Patient 1 and 2

| | Patient 1 | | | | Patient 2 |
|---|---|---|---|---|---|
| Ct Date: | Mar. 10, 2021 | Mar. 11, 2021 | Mar. 12, 2021 | Mar. 16, 2021 | March 2018 |
| N1 | 24.2 | 26.3 | 22.9 | 35.8 | 33.3 |
| N2 | 23.5 | 25.9 | 22.4 | 35.8 | 33.1 |
| RP | 21.7 | 22.9 | 20.8 | 22.5 | 24.4 |

TABLE 50
Panel of clinically relevant mutations in the S gene and corresponding
results from Patient 1 and 2. P/H indicates that Pt 2 was heterozygous
for the Wuhan and P681H variant. Mutations described on the CDC
website in “Science Brief: Emerging SARS-CoV-2 variants.”

| Mutations | Pt 1 | Pt 2 |
|---|---|---|
| 69/70 deletion | no | no |
| D80A | no | no |
| T95I | yes | yes |
| D138Y | no | no |
| 142-145 | yes (144 Del) | yes (G142V, 144Del) |
| R190T | no | yes |
| D215G | no | no |
| F220I | no | yes |
| R237K | no | yes |
| Δ242-244 | no | no |
| R246T | no | yes |
| D253G | no | no |
| K417T | no | undet |
| L452R | no | undet |
| S477N | no | undet |
| E464K | yes | undet |
| N501Y | no | undet |
| A570D | yes | no |
| D614G | yes | yes |
| H655Y | no | no |
| P681H | yes | P/H |
| A701V | no | undet |
| D796H | yes | undet |

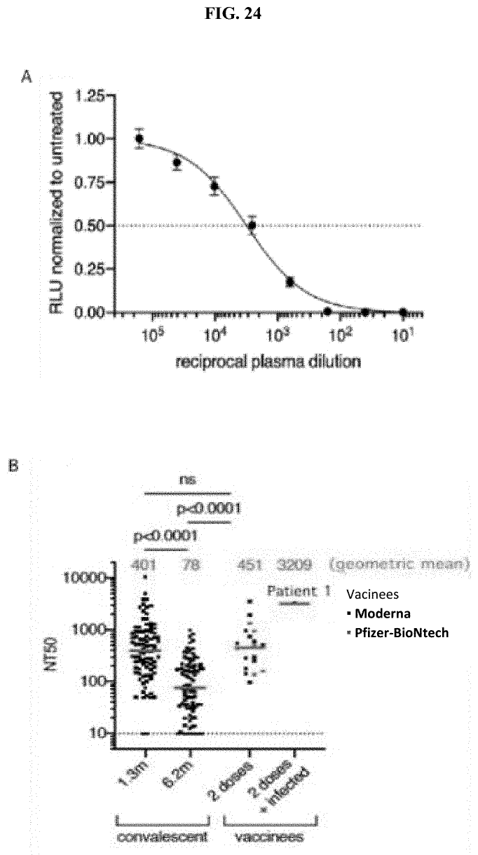

Serum obtained 4 days after the onset of Patient 1's symptoms was analyzed for neutralizing antibody to SARS-CoV-2 in a pseudotype neutralization assay [3]. These results revealed extremely high titers of neutralizing antibody (FIG. 24). These data, together with the clinical history, provide strong evidence that this individual likely had neutralizing antibodies elicited by vaccination.

To explore the variants in Patient 1 in more detail, we used excess RNA available from extracted saliva to undertake whole viral genome sequencing. This analysis confirmed the sequence changes found in the S gene by targeted amplification and suggested that the infection resulted from a SARS-CoV-2 variant that is related to but distinct from the known variants of concern, the UK variant B.1.1.7 and the NY variant B.1.526 (data not shown).

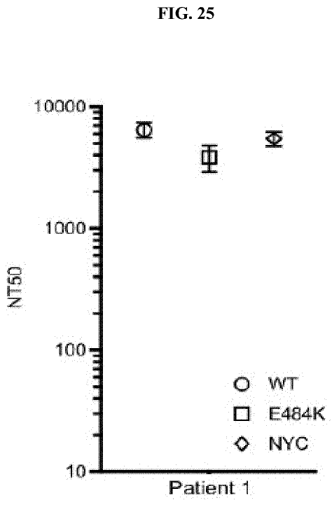

We therefore further tested the serum from Patient 1 to measure its effectiveness against the wild type, E484K mutant, and NYC variant and showed that the serum was equally effective against each (FIG. 25). These data suggest that Patient 1's antibody response recognized these variants but was nonetheless insufficient to prevent a breakthrough infection.

### Discussion

We describe 2 individuals who developed clinical symptoms of COVID-19 19- and 37-days after vaccination. Both patients had histories consistent with a clinical response to vaccine boost. In Patient 1 documented high titers of neutralizing antibody were present shortly after the development of symptoms. While a baseline antibody test before illness and after vaccine would have been ideal, it remains possible that she got infected before the booster shot taking full effect. Considering the clinical history, time course, and neutralizing antibody titers measured, we conclude that it is very likely that both patients had effective immune responses to their vaccines. While these cases presented with clinically mild symptoms, it will be of great importance to ascertain whether others can, or cannot, develop severe symptoms despite vaccination as variants continue to evolve [6]. Taken together, our observations support the conclusion that we have characterized bona fide examples of “vaccine breakthrough” manifesting in clinical symptoms. Moreover, data from Patient 1 indicates that infection with variant virus can be sustained with high viral load despite high levels of neutralizing antibody to variants.

Examination of the SARS-CoV-2 sequences from these patients revealed that both were infected with variant viruses. Rapid identification of sequence variants by targeted PCR amplification demonstrated that neither precisely fit any known Glade. Some of the Patient 1 substitutions (T95I, del144, E484K, A570D, D614G, P681H, D796H) were shared with B.1.526 (T95I, E484K, D614G7) and 3 were shared with Patient 2 (which had variants T95I, G142V and de1144, F220I, R237K, R246T, D614G). Whole viral genome sequencing revealed several additional substitutions, including D796H, present in a GC-rich region not identified by targeted PCR, that may decrease sensitivity to convalescent serum [8], as well as some unique non-coding changes compared to Wuhan, UK and NY clades. Although more detailed analysis of WGS from Patient 1 was undertaken, we could not conclude that it is a Pango lineage because it is only present in a single individual [9]. This revealed that its closest links on the phylogenetic tree were the “UK variant B.1.1.7 and the “NY variant” B.1.526, but with significant differences (data not shown). It will be of interest to determine whether this may have resulted from a recombination event between B.1.1.7 and B.1.526, as has been recently demonstrated for recombination between B.1.1.7 and the “wild type” Wuhan lineage [10]. Alternatively, shared substitutions may be the result of convergent evolution.

These observations in no way undermine the importance of the urgent efforts being taken at federal and state levels to vaccinate the US population. They also lend support to efforts to advance a new vaccine booster (as well as a pan-coronavirus vaccine) to provide increased protection against variants, as Moderna did in January 2021, announcing clinical efforts to target a new variant of SARS-CoV-2 that emerged in South Africa and includes three mutations (E484K, N501Y and K417N) in the ACE2 receptor binding domain. These efforts are of critical value since recent studies have shown that immunizations are proving to be less potent against the South African variant B.1.351, which might have acquired a partial resistance to neutralizing antibodies generated by natural infections or vaccinations [11,12]. At the same time, our observations underscore the importance of the ongoing “race” between immunization and the natural selection of potential viral escape mutants. During this critical period, our data support the need to maintain layers of mitigation strategies, including serial testing of asymptomatic individuals, open publication and analysis of vaccination and infection databases (such as those being accumulated in New York), and rapid sequencing SARS-CoV-2 RNA obtained from a variety of high risk individuals.

### REFERENCES

1. [No title] [Internet]. [cited 2021 Mar. 21]; Available from: https://wwwl.nyc.gov/assets/doh/downloads/pdf/covid/covid-19-data-variants-031621.pdf
2. Frank M, Blachere N E, Parveen S, et al. DRUL for School: Opening Pre-K with safe, simple, sensitive saliva testing for SAS-CoV-2.” medRxiv 2021, 254873.
3. Schmidt F, Weisblum Y, Muecksch F, et al. Measuring SARS-CoV-2 neutralizing antibody activity using pseudotyped and chimeric viruses. J Exp Med [Internet] 2020; 217(11). Available from: http://dx.doi.org/10.1084/jem.20201181
4. Wang Z, Schmidt F, Weisblum Y, et al. mRNA vaccine-elicited antibodies to SARS-CoV-2 and circulating variants. Nature [Internet] 2021; Available from: http://dx.doi.org/10.1038/s41586-021-03324-6
5. Weisblum Y, Schmidt F, Zhang F, et al. Escape from neutralizing antibodies by SARS-CoV-2 spike protein variants. Elife [Internet] 2020; 9. Available from: http://dx.doi.org/10.7554/eLife.61312
6. COVID-19 Vaccine Effectiveness Research [Internet]. 2021 [cited 2021 Apr. 1]; Available from: cdc.gov/vaccines/covid-19/effectiveness-research/protocols.html
7. CDC. Cases, Data, and Surveillance [Internet]. 2021 [cited 2021 Mar. 21]; Available from: https://www.cdc.gov/coronavirus/2019-ncov/cases-updates/variant-surveillance/variantinfo. html #Concern
8. Kemp S A, Collier D A, Datir R P, et al. SARS-CoV-2 evolution during treatment of chronic infection. Nature 2021; 1-10.
9. Rambaut A, Holmes E C, Hill V, et al. A dynamic nomenclature proposal for SARS-CoV-2 to assist genomic epidemiology [Internet]. Available from: http://dx.doi.org/10.1101/2020.04.17.046086
10. arambaut. Recombinant SARS-CoV-2 genomes involving lineage B.1.1.7 in the UK [Internet]. 2021 [cited 2021 Mar. 21]; Available from: virological.org/t/recombinantsars-cov-2-genomes-involving-lineage-b-1-1-7-in-the-uk/658
11. Stamatatos L, Czartoski J, Wan Y-H, et al. mRNA vaccination boosts cross-variant neutralizing antibodies elicited by SARS-CoV-2 infection. Science [Internet] 2021; Available from: http://dx.doi.org/10.1126/science.abg9175
12. Planas D, Bruel T, Grzelak L, et al. Sensitivity of infectious SARS-CoV-2 B.1.1.7 and B.1.351 variants to neutralizing antibodies. Nat Med [Internet] 2021; Available from: http://dx.doi.org/10.1038/s41591-021-01318-5
13. Luchsinger L L, Ransegnola B P, Jin D K, et al. Serological Assays Estimate Highly Variable SARS-CoV-2 Neutralizing Antibody Activity in Recovered COVID-19 Patients. J Clin Microbiol [Internet] 2020; 58(12). Available from: http://dx.doi.org/10.1128/JCM.02005-20
14. Robbiani D F, Gaebler C, Muecksch F, et al. Convergent antibody responses to SARS-CoV-2 in convalescent individuals. Nature 2020; 584(7821):437-42.
15. Gaebler C, Wang Z, Lorenzi J C C, et al. Evolution of antibody immunity to SARS-CoV-2. Nature [Internet] 2021; Available from: dx.doi.org/10.1038/s41586-021-03207-w

### Example 10

### Procedure for Pooling Specimens and Pooling Sample Analysis

In order to open business, schools, hospitals and other facilities and centers where employees, students, teachers, patients etc need to report to work, school, employment etc it has been desirable to have available a rapid and straightforward test for COVID-19. The saliva test provides a particularly desirable option. Individuals can collect the sample themselves, without risking possible exposure to any medical or testing personel with sample collection. Also, the ease of sample collection and rapid results from testing improve, ensure and enhance the cooperation and compliance of individuals, employees, students, personnel, patients, visitors etc.

In instances where the rate of infection is relatively low, for instance on the order of 10% of the population or less, even on the order of 5% or even on the order of 1%, or less than 1%, particularly wherein individuals, employees, students, personnel, patients, visitors etc are being regularly tested, such as daily, bi-weekly, every 2-4 days, weekly, every 7-10 days, every 10-14 days, every two weeks, monthly, the cost of testing can be reduced and the efficiency of testing can be increased by pooling samples from multiple individuals and testing a pooled sample. Thus, if the percentage of positive virus result is low, such as in instances wherein the expected the rate of infection is relatively low, for instance on the order of 10% of the population or less, even on the order of 5% or even on the order of 1%, or less than 1%, samples collected from several, numerous, ten, dozens, hundreds, thousands of individuals can be pooled together and analyzed in a single test assay or in multiple overlapping assay sets. Sample saliva from multiple individuals can be pooled together, nucleic acid, particularly RNA isolated from the pooled sample. PCR is conducted on the RNA isolated from the pooled sample with suitable primers and probes and the result determined. A negative result of a SARS-CoV-2 assay from the pooled sample indicates that all of the individuals are negative for the SARS-CoV-2 virus and that none of the individuals are positive for the SARS-CoV-2 virus. A positive result indicates that at least one individual whose sample was included in the pooled sample is positive for the SARS-CoV-2 virus.

Alternatively, nucleic acid, particularly RNA, from multiple individuals can be pooled together after isolation of the nucleic acid, PCR is conducted on the pooled RNA with suitable primers and probes and the result determined. A negative result of a SARS-CoV-2 assay from the pooled sample indicates that all of the individuals are negative for the SARS-CoV-2 virus and that none of the individuals are positive for the SARS-CoV-2 virus. A positive result indicates that at least one individual whose sample was included in the pooled sample is positive for the SARS-CoV-2 virus.

The following provides a procedure and approach for successfully pooling and testing for SARS-CoV-2.

Pooling Strategy for SARS-CoV-2 Saliva Testing with DRUL2

To scale our testing, we have developed a novel pooling strategy. Pooling saliva has been implemented by others, but suffers from decreased sensitivity as pool sizes increase (Watkins A E et al “Pooling Saliva to Increase SARS-CoV-2 Testing Capacity” 2020 Sep. 3; doi:10.1101/2020.09.02.20183830), and increased risk of manipulating and transporting infectious saliva pooling strategies are in some cases being done on site (Coronavirus Testing Guidance 2020). We have taken a new approach that leverages the power of the DRUL-based sensitivity assays, logistical safety and accompanying technology to allow a breakthrough in scaling without loss of sensitivity. This in turn has led to development of a new strategy for pooling, in which additive sampling of subsets of pools leads to multiplicative power in precise detection of rare positives in large sample sets.

Background: Pooling Saliva Samples

We have used the DRUL2 purification protocol to develop a new strategy for pooling up to 96 saliva samples at a time with almost no loss in sensitivity. We have combined this with a combinatorial strategy that allows pooling over many samples, returning results without significant loss of sensitivity, with extremely high specificity. We find that this strategy can pinpoint a single positive individual from among 1,000 samples, with no retesting or confirmation required. This approach has the potential to be further scaled, allowing the potential to detect hundreds of thousands or even a million samples per day with an appropriately equipped robotic lab and logistical supply line.

The essence of this strategy is to take a different approach to pooling, leveraging our success in the DRUL2 development protocols. DRUL2 buffer and protocols yield exceptionally pure RNA from individual saliva samples, with great sensitivity for SARS-CoV-2 (0.5 viral copies/ul; 10× greater than assays for SARS-CoV-2 detection from saliva developed at Yale). Our strategy is to pool this RNA (rather than saliva or NPS samples). This RNA can then be re-concentrated, using appropriate lab-developed buffers and protocols, into a concentrated RNA.

We have found that we can pool up to 96 such “concentrated” RNA samples down to the volume typically used to assay for SARS-CoV-2 from a single individual. Re-running this RNA by multiplex PCR has been found to show increased sensitivity for RP RNA (consistent with the concept that the number of RNA molecules in 96 individuals is greater than the amount in a single individual), demonstrating the efficiency and lack of saturation of RNA purification present in our DRUL2 protocol.

More importantly, we have also found that re-running this 96-x pooled RNA (from 96 individuals) in which one sample is positive for SARS-Cov-2 and 95 are negative, leads to nearly equal sensitivity for SARS-CoV-2 detection as seen in the original sample. Such consistency of SARS-CoV-2 detection is true across a wide range of 2-fold sample dilutions from 1× (undiluted) individual RNA samples to 96× dilutions. This opens a new method and strategy for efficient detection of COVID-19 infection in “1-in 100” individuals, and offers the possibility of greatly increased throughput and concomitant savings in time and expense for scaled detection.

In addition, we have developed a means of detecting an even more sensitive strategy, in which multiple pools of 100 individual samples can be resampled in an additive strategy that gives multiplicative power. This strategy, explained below, is able to detect one positive in 1,000 samples. Implications of this new strategy again are even more important for throughput and savings.

Pooling Saliva Protocol: Results and Methods

Validation: Pooling SARS-CoV-2 Saliva Laboratory Developed Test

Reagents

Pooling SARS-CoV-2 Saliva samples begins not with pooling saliva, but with pooling purified RNA from saliva of individual samples. For example RNA may be purified using the DRUL2 saliva testing protocol. The purified RNA obtained using the DRUL2 saliva testing protocol is eluted in a final volume of 35-50 ul. In the usual (non-pooled) protocol, 5-10 ul of an individual's purified RNA is then used in a single multiplex PCR assay that determines the presence, if any, of SARS-CoV-2 RNA and its relative abundance. The presence of SARS-CoV-2 RNA is assessed using primers specific to the N gene (two individual primer sets, N1 and N2), and a positive control that confirms the saliva RNA extraction and subsequent PCR reaction worked is assessed using primers specific to the RP gene. The latter is essential to determine that the sample can adequately be determined as negative for SARS-CoV-2.

For pooling samples of purified RNA, prior to PCR assay, multiple 5-10 ul aliquots from individual purified RNAs are pooled into one well. These pooled RNA samples can directly be added to a modified binding buffer, magnetic beads, and reconcentrated using a modified Apex or Presto extraction procedure presented below.

Reagent Preparation for Pooling

To make 100 ml of DRUL2× Binding Buffer:

TABLE 51
2X Binding Buffer.

| | | |
|---|---|---|
| Reagent | Amount needed | Final Concentration |

| PEG 8000 | 10 | g | 20% | (w/v) |
|---|---|---|---|---|
| 5M NaCl | 46 | ml | 4.5M | |
| 1.0M Trisodium Citrate | 1 | ml | 2 | mM |
| Tween 20 | 0.050 | ml | 0.05% | (v/v) |
| 0.1N HCl | 0.056 | ml | | |

| | | | |
|---|---|---|---|
| Nuclease-free water | 4 | ml | — |

Purified RNA obtained using the DRUL2 saliva testing protocol (above) is eluted in a final volume of 35 ul

To pool 96 samples of purified RNA (note that smaller pool sizes (e.g. 12, 24, 48× pools) can also be used)

TABLE 52
Making RNA pools

| | Amount RNA/ | |
|---|---|---|
| Reagent | individual | Total Pooled RNA |
| 1 RNA purified from | 8.7 ul (range: 6.5 | 8.7 ul (matched to |
| 1 individuals | to 10 ul) | range of pooled RNA |
| to be re-tested | | used |
| 96 RNAs purified from | 6.5 ul (range: up | 624 ul for 6.5 ul RNA |
| 96 individuals | to 10 ul) | 835 ul for 8.7 ul RNA |
| to be tested | | etc |

To prepare Pooled RNA for Magnetic Bead Purification Binding Buffer:

TABLE 53
RNA + Binding buffer + Magnetic
Beads for re-extraction of 96X pool

| | DRUL2X | | CleanNGS | |
|---|---|---|---|---|
| | Binding | Linear | magnetic | Final |
| RNA input | Buffer | Acrylamide | beads | Volume |
| 96X RNA; e.g. 96 × | 60 ul | 2 ul | 110 ul | 1,007 ul |
| 8.7 ul = 835 ul total | | | | |

Equipment and Materials

Scienceware® pipette V-bottom reservoir, 12 wells, 5 ml each Cat #Z370843

KingFisher Apex or Presto Pooled Sample Extraction Procedure
1. Prepare one KingFisher Deep-well plates by adding the appropriate amount of 2×BB, linear acrylamide and CleanNGS magnetic beads to each well of what is now named the “Master RNA Re-extraction Plate.” Per Table 22; volume will be 172 ul per well. Set aside.
2. Pooling 96-well Sample Tray 1.
   a. Prepare 96 samples of RNA into 96 wells exactly as in KingFisher Extraction Procedure, steps 1-9, page 35. At step 10 (now renamed step 10P), rather than removing 10-15 ul of eluted RNA for PCR, pool 96 RNAs in triplicate (3 dimensions: plate, row, column) as follows in steps 3-4.
3. Use liquid handler to remove triplicate volumes (e.g. 3×8.7 ul)) from each well of RNA purified previously (“RNA to be pooled Master Plate” from step 10: page 35) and hold RNA temporarily in this pipet tip.
   i. For example, each pipet tip will have 3×8.7 ul or 26.1 ul RNA separately from each well of the 96 well plate.
4. Dimension 1: Plate Pooling
   a. Release 1/3 of the RNA (e.g. 8.7 ul from each of the 96 wells; total volume released=96×8.7 ul=835 ul) into a single v-shape tray.
   b. Mix RNA by swirling gently and collect entire volume with single pipette into a single well of the Master RNA Re-extraction Plate.
   c. Mix in order to mix the viscous solution. Mix gently so as not to introduce bubbles.
   d. Keep the Master RNA Re-extraction Plate cold until Pooled PCR step.
5. Master RNA Pooling Trays for Dimension 2-3.
   a. Release the remaining ⅔ of the RNA present in liquid handler pipet tip (step 3; e.g. 17.4 ul from each of the 96 wells into the RNA to be pooled Master Plate (a single master 96 well tray); keep cold (4° C.).
6. Repeat Pooling Steps for additional 96 well Sample Trays, e.g. Sample Tray 2 up to Sample Tray “N”. We set N=10 here for the purposes of illustration.
   a. For example, for Pooling 96-well Sample Tray 2, take RNA eluted in step 10P from Tray 2 and remove triplicate volumes of RNA using a liquid handler (Step 3). Collect Dimension 1 (Plate Pooling) into well 2 of the Master RNA Re-extraction Plate.
   b. Release the remaining ⅔ of the RNA present in liquid handler pipet tip from Sample Tray 2 to the same RNA to be pooled Master Plate from Step 3.
      i. The volume in each well will increase by the amount of RNA present in the remaining ⅔ of the liquid handler pipet tip (step 3; e.g. 17.4 ul).
   c. Repeat steps 6a-6b until a maximum of 10 Sample Trays have been pooled in Dimension 1 into the Master RNA Re-extraction Plate, which will now have 10 wells filled.
7. Row and Column Pooling into the Master RNA Re-extraction Plate
   a. The RNA to be pooled Master Plate will now have N (in this case 10) pooled samples of 2×RNA (17.4 ul×10=174 ul in this example) in each well at 4° C.
8. Dimension 2: Pooling Columns
   a. With a multichannel pipet/liquid handler, remove one-half the RNA volume in each of the samples in column 1 (which will comprise pooling of 8 samples, each with 174 ul from 10 plates) into a single v-shape tray.
   b. Mix RNA by swirling gently and collect entire volume with single pipette into a unused single well of the Master RNA Re-extraction Plate. This well will have pre-aliquoted 2×BB, linear acrylamide and magnetic beads in 172 ul (step 1). The final volume will be 174 ul+172 ul.
   c. Mix in order to mix the viscous solution. Mix gently so as not to introduce bubbles.
   d. Repeat steps 8a-8c with column 2-column 12 of the RNA to be pooled Master Plate. Importantly, each pooled column 2-12 will be aliquoted separately into 11 individual wells on the Master RNA Re-extraction Plate.
      i. Note that the RNA to be pooled Master Plate will now have the remaining ⅓^{rd} of each pooled RNA in each well.
   e. Keep the Master RNA Re-extraction Plate cold until Pooled PCR step.
9. Dimension 3: Pooling Rows
   a. With a multichannel pipet/liquid handler, remove the remaining RNA volume in each of the samples in row 1 (which will comprise pooling of 12 samples, each with 174 ul from 10 plates) into a single v-shape tray.
   b. Mix RNA by swirling gently and collect entire volume with single pipette into a unused single well of the Master RNA Re-extraction Plate. This well will have pre-aliquoted 2×BB, linear acrylamide and magnetic beads in 172 ul (step 1). The final volume will be 174 ul+172 ul.
   c. Mix in order to mix the viscous solution. Mix gently so as not to introduce bubbles.
   d. Repeat steps 9a-9c with row 2-row 8 of the RNA to be pooled Master Plate.
      i. Note that the RNA to be pooled Master Plate will now have no remaining RNA in each well.
         1. To save plastic, this plate can be reused by soaking in 1% sodium hypochlorite for 10 min at room temperature, rinsed thoroughly with distilled water for 10 minutes and dried.
10. Re-extraction and Analysis of pooled RNAs:
   a. The completed Master RNA Re-extraction Plate is now analyzed in the Pooled PCR step.
   b. Pooled PCR step. The PCR Master RNA Re-extraction Plate plate setup and qPCR run are undertaken as described in Example 3.

Data Analysis and Interpretation
1. Controls on each plate are analyzed first and the run is QC'd as acceptable or not according to Table 14 on page 36.
2. Interpretation of Sample Results is performed as described in Example 3.
3. Unique features of N-dimensional pooling analysis:
   a. Each true positive will be present in triplicate: in one plate, one row and one column.
      i. This gives a means of conferring confidence in each result, as N1, N2 and RP values should match
      ii. In addition, if two positives are present on the Master RNA Re-extraction plate, they may be distinguished from each other definitively if the triplicate values of N1, N2 and RP and sufficiently distinct from a first positive.
   b. This strategy offers the possibility of identifying one or more positives with extremely high confidence from a single PCR runoff the Master RNA Re-extraction Plate. In this case, no additional “confirmatory” re analysis of RNA from a putative positive may be required, allowing rapid mobilization of notification and contact tracing.

Assay Performance of Pooling Strategy for SARS-CoV-2 Saliva Testing with DRUL2

Saliva samples from up to 96 individual samples were tested for the presence of SARS-CoV-2 using our Standard DRUL2 EUA approved Saliva protocol. All samples were negative.

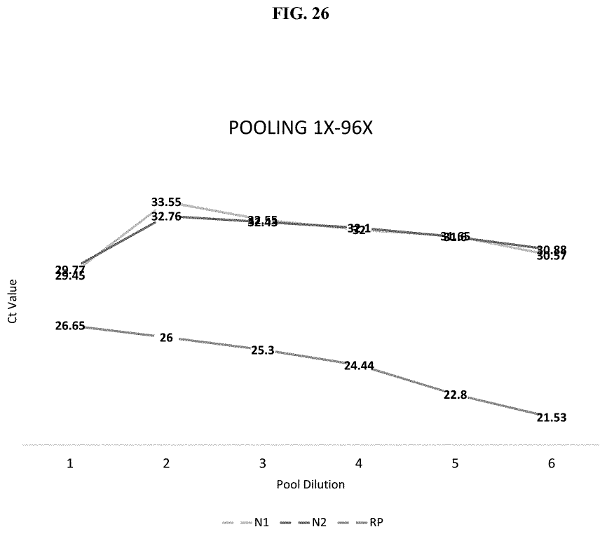

A single sample was then spiked with 10 copies/ul of chemically inactivated SARS-CoV-2 virus (Zeptometrix). 10 microliters of this sample was then tested for RNA by RT-PCR, with N1/N2 values of 29.4/29.77, RP 26.65. This positive sample was then diluted with additional specimens of saliva RNA purified by DRUL2 methods described in Example 3. Results from multiple experiments were obtained, and a representative pooling experiment is shown in FIG. 26.

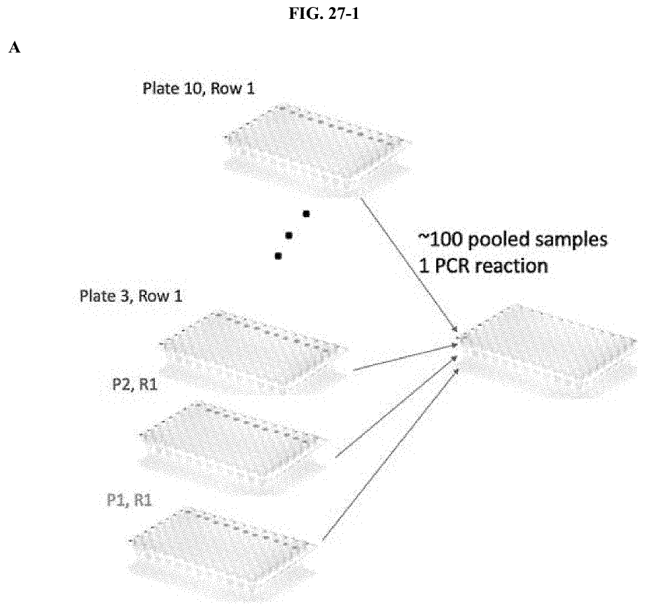

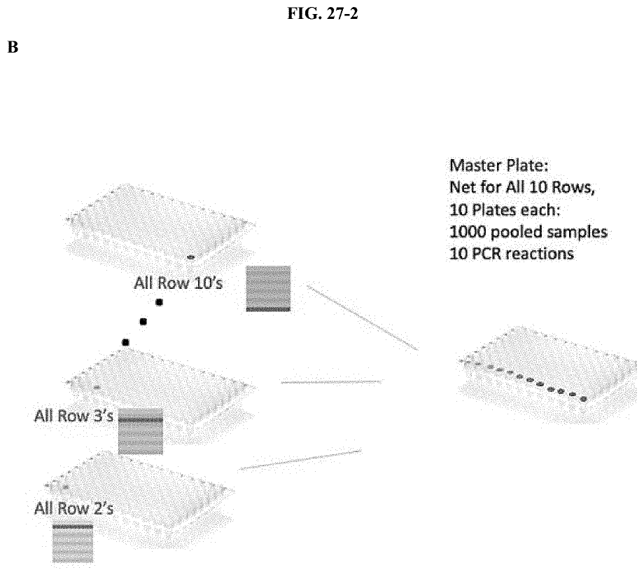

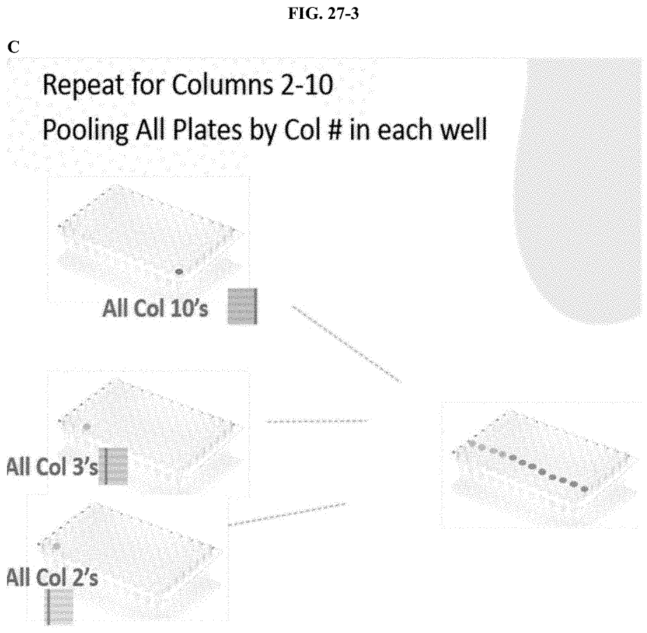

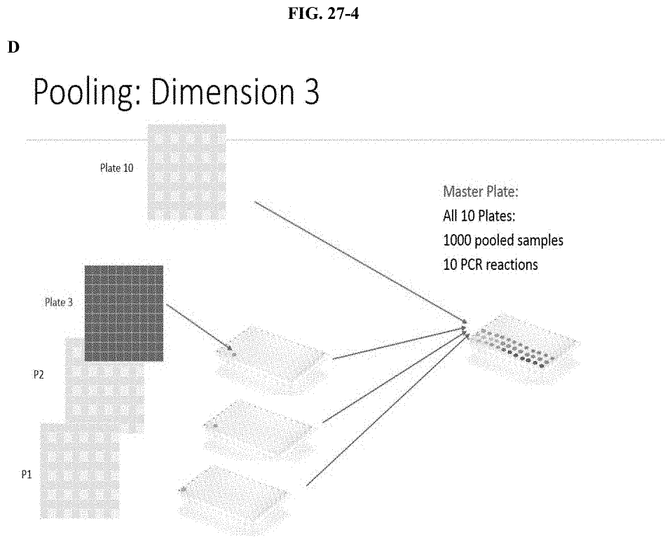

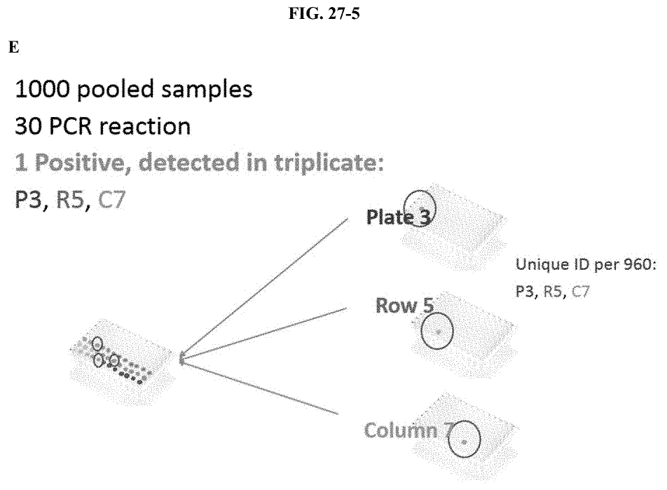

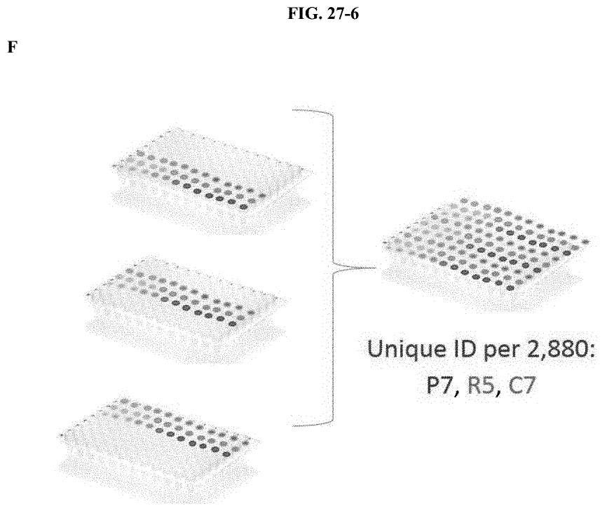

The pooling approach includes an N-dimensional pooling strategy, whereby pooling of samples can be dimensionally increased and adjusted to accommodate increased numbers of samples for pooling with satisfactory sensitivity and results. FIG. 27 provides illustrations of N-dimensional pooling strategy for SARS-CoV-2 testing.

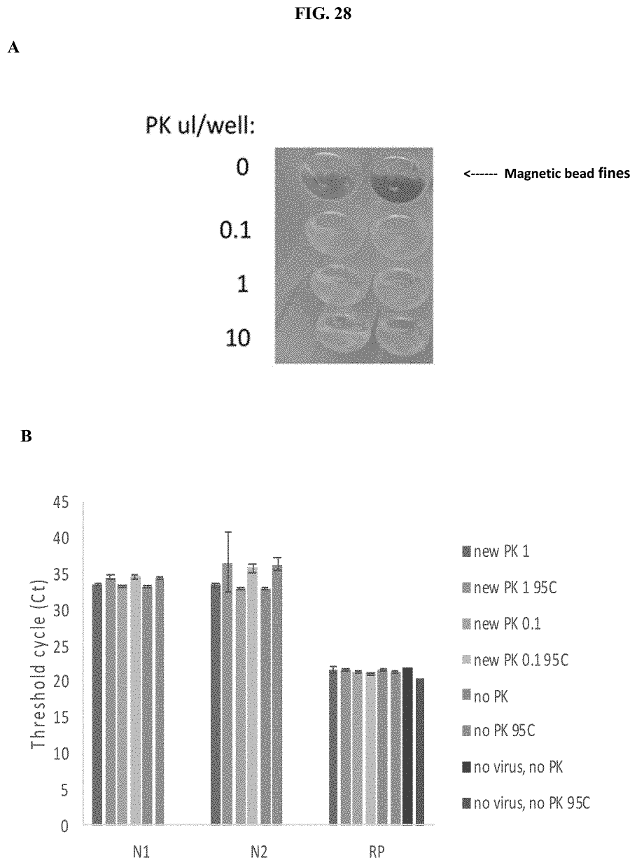

It has been recognized that proteinase K treatment of samples improves the results in a pooling strategy and approach (FIG. 28). Proteinase K can be diluted after use and treatment or can be heat inactivated. Dilution is sufficient and in some aspects may be preferred.

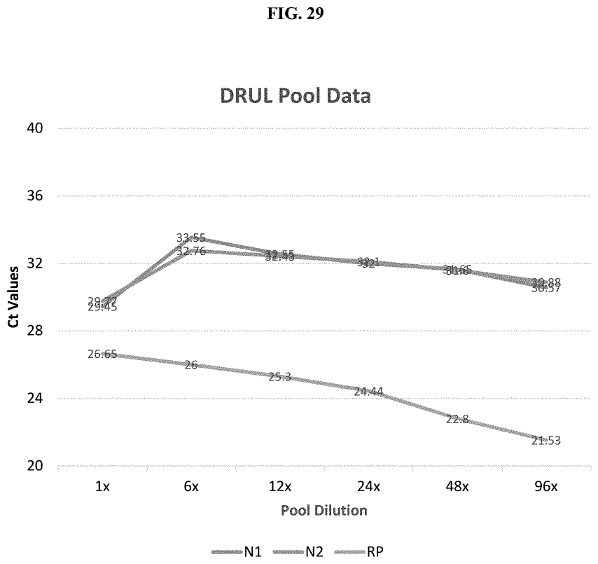

Pooling results from 2,880 individuals are depicted in FIG. 29, showing approximately 97% RNA recovery with the pooling method. In fact, RP signal improves with pooling (lower Ct), indicating that concentration of saliva RNA is working and effective to maintain sensitivity.

### REFERENCES

1. Liu L, Wei Q, Alvarez X, Wang H, Du Y, Zhu H, et al. Epithelial cells lining salivary gland ducts are early target cells of severe acute respiratory syndrome coronavirus infection in the upper respiratory tracts of rhesus macaques. J Virol. 2011; 85: 4025-4030.
2. To K K W, Lu L, Yip C C Y, Poon R W S, Fung A M Y, Cheng A, et al. Additional molecular testing of saliva specimens improves the detection of respiratory viruses. Emerging Microbes & Infections. 2017. pp. 1-7. doi:10.1038/emi.2017.35
3. To K K-W, Tsang O T-Y, Yip C C-Y, Chan K-H, Wu T-C, Chan J M-C, et al. Consistent Detection of 2019 Novel Coronavirus in Saliva. Clin Infect Dis. 2020; 71: 841-843.
4. Vogels C B F, Watkins A E, Harden C A, Brackney D, Shafer J, Wang J, et al. SalivaDirect: A simplified and flexible platform to enhance SARS-CoV-2 testing capacity. doi:10.1101/2020.08.03.20167791
5. Wong R C, Wong A H, Ho Y I, Leung E C, Lai R W. Performance evaluation of Panther Fusion SARS-CoV-2 assay for detection of SARS-CoV-2 from deep throat saliva, nasopharyngeal, and lower-respiratory-tract specimens. Journal of Medical Virology. 2020. doi:10.1002/jmv.26574
6. Tutuncu E, Ozgur D, Karamese M. Saliva Samples for Detection of SARS-CoV-2 in Mildly Symptomatic and Asymptomatic Patients. SSRN Electronic Journal. doi:10.2139/ssrn.3646109
7. Caulley L, Corsten M, Eapen L, Whelan J, Angel J B, Antonation K, et al. Salivary Detection of COVID-19. Annals of Internal Medicine. 2020. doi:10.7326/m20-4738

This invention may be embodied in other forms or carried out in other ways without departing from the spirit or essential characteristics thereof. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

1. A method for profiling and analysis of infectious agent RNA, particularly viral RNA, of saliva samples from one or more patient or individual comprising:
(a) obtaining one or more small volume saliva sample, wherein the sample is collected in or otherwise combined with a lysis/RNA stabilization buffer comprising guanidinine thiocyanate (GSCN), whereby infectious agent, particularly virus, is inactivated and RNA is stabilized;
(b) isolating RNA;
(c) reverse transcribing the RNA to generate cDNA;
(d) specifically amplifying any infectious agent RNA, particularly viral RNA, using primers and probes specific for the target infectious agent or virus; and
(e) determining the presence and identity of infectious agent RNA, particularly viral RNA, whereby primer amplified nucleic acid for the target infectious agent or virus is identified and characterized.

2. The method of claim 1 wherein the saliva sample volume is about 200-4000.

3. The method of claim 1, wherein the saliva sample is combined with an approximately equal amount of lysis/RNA stabilization buffer.

4. The method of claim 1, wherein the lysis/RNA stabilization buffer comprises guanidinine thiocyanate (GSCN), mild detergent and organic sodium salt.

5. The method of claim 1, wherein the lysis/RNA stabilization buffer has a concentration of components comprising GSCN in the range of 5-6 M, sarkosyl in a % range of 0.2% to 0.5% and sodium acetate (NaAc) at 150 mM-200 mM, and a pH of 5-6.

6. The method of claim 1, wherein the saliva and lysis/RNA stabilization buffer are heated briefly for about 10-20 minutes at 65° C. prior to isolating RNA.

7. The method of claim 1, wherein a protein denaturant or reducing agent is added to the sample in lysis/RNA stabilization buffer prior to isolating RNA.

8. The method of claim 7, wherein the protein denaturant or reducing agent is selected from 2-mercaptoethanol, dithiothreitol (DTT), and TCEP (tris(2-carboxymethyl).

9. The method of claim 1 for evaluation of coronavirus SARS-CoV-2.

10. The method of claim 9, wherein the method utilizes the probes and primer sequences provided in Table 1, Table 28, or in Table 48.

11. The method of claim 1, wherein the saliva sample volume is less than 500 μl, less than 300 μl, less than 250 μl, about 200-300 μl, less than 200 μl, about 100-300 μl, about 150-300 μl, about 100-250 μl, about 300 μl.

12. The method of claim 1, wherein the saliva and lysis/RNA stabilization buffer is combined with a protease prior to or during RNA isolation, wherein the protease is proteinase K.

13. The method of claim 1, wherein RNA is isolated by a method or system selected from phenol extraction, column-based isolation, precipitation, or magnetic bead based purification.

14. A system or kit for viral RNA profiling and analysis of saliva samples from a patient or individual comprising:
(a) a means for self-collection of a saliva sample by the patient or individual or by a non-medical person, comprising a receptable for a saliva drool or spit;
(b) a tube or receptacle for receiving the saliva sample on collection and containing a volume of lysis/RNA stabilization solution whereby virus in the sample is inactivated and RNA is stabilized; and
(c) one or more appropriate label(s) for designating the name or identity of the patient or individual, date of sample collection and time of sample collection.

15. The system or kit of claim 14, further comprising an envelope or mailing container for shipment of the sample to a laboratory or facility for RNA isolation and analysis.

16. The system or kit of claim 14, for viral RNA profiling and analysis of multiple saliva samples collected in series from numerous patients or individuals comprising:
(a) a set of numerous means for self-collection of individual saliva samples by one or more or multiple patients or individuals, each comprising a receptable for a saliva drool or spit;
(b) a set of numerous tubes each individually for receiving a saliva sample on collection and containing a volume of lysis/RNA stabilization solution whereby virus in the sample is inactivated and RNA is stabilized;
(c) numerous appropriate label(s) for designating the name or identity of the patient(s) or individual(s), date of sample collection and time of sample collection; and
(d) numerous envelopes or mailing containers for shipment of each sample or several samples to a laboratory or facility for RNA isolation and analysis.

17. The system or kit of claim 14, wherein the volume of lysis/RNA stabilization solution is less than 1 ml, about 500 μl or less, about 300 μl or less, about 200-300 μl, about 250 μl, about 300 μl.

18. The system or kit of claim 14, wherein the tube or receptacle for receiving the small volume sample and containing lysis/RNA stabilization solution has a total volume capacity of 1.5 ml or less, 1.2 ml or less, or 1 ml or less.

19. The system or kit of claim 14, wherein the lysis/RNA stabilization buffer comprises guanidinine thiocyanate (GSCN), mild detergent and organic sodium salt.

20. The system or kit of claim 14, wherein the lysis/RNA stabilization buffer has a concentration of components comprising GSCN in the range of 5-6 M, sarkosyl in a % range of 0.2% to 0.5% and sodium acetate (NaAc) at 150 mM-200 mM, and a pH of 5-6.

21. A method for pooling RNA for profiling and analysis of infectious agent RNA, particularly viral RNA, of saliva samples from numerous, dozens or hundreds of patients or individuals comprising:
(a) obtaining one or more small volume saliva sample, wherein the sample is collected in or otherwise combined with a lysis/RNA stabilization buffer comprising guanidinine thiocyanate (GSCN), mild detergent and organic sodium salt whereby infectious agent, particularly virus, is inactivated and RNA is stabilized;
(b) heating the saliva and lysis/RNA stabilization buffer sample briefly at about 65° C.;
(c) adding a protein denaturant and/or reducing agent to the heated sample;
(d) adding a binding buffer suitable to promote binding of RNA nucleic acid to a column or magnetic beads;
(e) isolating RNA to generate numerous individual saliva isolated RNA samples;
(f) combining aliquots of numerous individual saliva isolated RNA samples to generate a pooled RNA sample;
(g) adding a binding buffer having a 2× salt (such as each of NaCl and trisodium citrate) concentration compared to the binding buffer of (d);
(h) isolating RNA using magnetic beads to generate a reconcentrated pooled RNA sample;
(i) reverse transcribing the RNA to generate cDNA;
(j) specifically amplifying any infectious agent RNA, particularly viral RNA, using primers and probes specific for the target infectious agent or virus; and
(k) determining the presence and identity of infectious agent RNA, particularly viral RNA, whereby primer amplified nucleic acid for the target infectious agent or virus is identified and characterized.

22. The method of claim 21 wherein protease, particularly proteinase K is added to the binding buffer having a 2× salt concentration in (g).

23. The method of claim 21, wherein the protein denaturant or reducing agent is selected from 2-mercaptoethanol, dithiothreitol (DTT), and TCEP (tris(2-carboxymethyl).

24. The method of claim 21, wherein aliquots of up to 96 individual saliva isolated RNA samples are combined to generate a pooled RNA sample.

25. The method of claim 21, wherein a single positive sample can be detected in a pooled RNA sample comprising 96 or 100 individual isolated RNA samples.


SEQUENCE LISTING (62 sequences)

SEQ ID NO: 1 - DNA, 20 bp, Artificial Sequence
  N1 forward primer
gaccccaaaatcagcgaaat

SEQ ID NO: 2 - DNA, 24 bp, Artificial Sequence
  N1 reverse primer
tctggttactgccagttgattctg

SEQ ID NO: 3 - DNA, 24 bp, Artificial Sequence
  N1 probe
accccgcattacgtttggtggacc

SEQ ID NO: 4 - DNA, 20 bp, Artificial Sequence
  N2 forward primer
ttacaaacattggccgcaaa

SEQ ID NO: 5 - DNA, 18 bp, Artificial Sequence
  N2 reverse primer
gcgcgacattccgaagaa

SEQ ID NO: 6 - DNA, 23 bp, Artificial Sequence
  N2 probe
acaatttgcccccagcgcttcag

SEQ ID NO: 7 - DNA, 22 bp, Artificial Sequence
  N3 forward primer
gggagccttgaatacaccaaaa

SEQ ID NO: 8 - DNA, 21 bp, Artificial Sequence
  N3 reverse primer
tgtagcacgattgcagcattg

SEQ ID NO: 9 - DNA, 24 bp, Artificial Sequence
  N3 probe
aycacattggcacccgcaatcctg

SEQ ID NO: 10 - DNA, 18 bp, Artificial Sequence
  RNAseP forward primer
agatttggacctgcgagc

SEQ ID NO: 11 - DNA, 18 bp, Artificial Sequence
  RNAseP reverse primer
gagcggctgtctccacaa

SEQ ID NO: 12 - DNA, 23 bp, Artificial Sequence
  RNAseP probe
ttctgacctgaaggctctgcgcg

SEQ ID NO: 13 - DNA, 23 bp, Artificial Sequence
  N1 forward primer
gaccaggaactaatcagacaagg

SEQ ID NO: 14 - DNA, 19 bp, Artificial Sequence
  N1 reverse primer
tcaaccacgttcccgaagg

SEQ ID NO: 15 - DNA, 25 bp, Artificial Sequence
  N1 probe
cgacattccgaagaacgctgaagcg

SEQ ID NO: 16 - DNA, 25 bp, Artificial Sequence
  N2 forward primer
gccatcaaattggatgacaaagatc

SEQ ID NO: 17 - DNA, 21 bp, Artificial Sequence
  N2 reverse primer
taggctctgttggtgggaatg

SEQ ID NO: 18 - DNA, 27 bp, Artificial Sequence
  N2 probe
cattttgctgaataagcatattgacgc

SEQ ID NO: 19 - DNA, 19 bp, Artificial Sequence
  RNAseP forward primer
agatttggacctgcgagcg

SEQ ID NO: 20 - DNA, 20 bp, Artificial Sequence
  RNAseP reverse primer
gagcggctgtctccacaagt

SEQ ID NO: 21 - DNA, 23 bp, Artificial Sequence
  RNAseP probe
ttctgacctgaaggctctgcgcg

SEQ ID NO: 22 - DNA, 22 bp, Artificial Sequence
  forward primer
tgaagatgcttgtactgtggct

SEQ ID NO: 23 - DNA, 20 bp, Artificial Sequence
  reverse primer
ctgtcatgttgctcatgggg

SEQ ID NO: 24 - DNA, 22 bp, Artificial Sequence
  forward primer
agatgcttgtactgtggcttct

SEQ ID NO: 25 - DNA, 21 bp, Artificial Sequence
  reverse primer
gtcatgttgctcatgggggag

SEQ ID NO: 26 - DNA, 23 bp, Artificial Sequence
  N1 forward primer
gaccaggaactaatcagacaagg

SEQ ID NO: 27 - DNA, 19 bp, Artificial Sequence
  N1 reverse primer
tcaaccacgttcccgaagg

SEQ ID NO: 28 - DNA, 25 bp, Artificial Sequence
  N1 probe
cgacattccgaagaacgctgaagcg

SEQ ID NO: 29 - DNA, 25 bp, Artificial Sequence
  N2 forward primer
gcatccaaattggatgacaaagatc

SEQ ID NO: 30 - DNA, 21 bp, Artificial Sequence
  N2 reverse primer
taggctctgttggtgggaatg

SEQ ID NO: 31 - DNA, 27 bp, Artificial Sequence
  N2 probe
cattttgctgaataagcatattgacgc

SEQ ID NO: 32 - DNA, 19 bp, Artificial Sequence
  RNAseP forward primer
agatttggacctgcgagcg

SEQ ID NO: 33 - DNA, 20 bp, ARtificial Sequence
  RNAseP reverse primer
gagcggctgtctccacaagt

SEQ ID NO: 34 - DNA, 23 bp, Artificial Sequence
  RNAseP probe
ttctgacctgaaggctctgcgcg

SEQ ID NO: 35 - DNA, 21 bp, Artificial Sequence
  forward primer
ccagatgattttacaggctgc

SEQ ID NO: 36 - DNA, 23 bp, Artificial Sequence
  reverse primer
ctactgatgtcttggtcatagac

SEQ ID NO: 37 - DNA, 21 bp, Artificial Sequence
  forward primer
cttgttttattgccactagtc

SEQ ID NO: 38 - DNA, 23 bp, Artificial Sequence
  forward primer
caagtggggtaaggctagacttt

SEQ ID NO: 39 - DNA, 22 bp, Artificial Sequence
  reverse primer
acttaggataatcccaacccat

SEQ ID NO: 40 - DNA, 30 bp, Artificial Sequence
  forward primer
cctactaaattaaatgatctctgctttact

SEQ ID NO: 41 - DNA, 21 bp, Artificial Sequence
  reverse primer
caagctataacgcagcctgta

SEQ ID NO: 42 - DNA, 22 bp, Artificial Sequence
  influenza A forward primer
gaccratcctgtcacctctgac

SEQ ID NO: 43 - DNA, 24 bp, Artificial Sequemc
  Influenza A reverse primer
agggcattytggacaaakcgtcta

SEQ ID NO: 44 - DNA, 24 bp, Artificial Sequence
  Influenza A probe
tgcagtcctcgctcactgggcacg

SEQ ID NO: 45 - DNA, 21 bp, Artificial Sequence
  Influenza B forward primer
acaattgcctacytgctttca

SEQ ID NO: 46 - DNA, 19 bp, Artificial Sequence
  Influenza B reverse primer
tctttcccaccraaccaac

SEQ ID NO: 47 - DNA, 30 bp, Artificial Sequence
  Influenza B probe
agaagatggagaargcaaagcagaactagc

SEQ ID NO: 48 - DNA, 30 bp, Artificial Seuqnce
  human metapneumovirus forward primer
catayaarcatgctatattraaagagtctc

SEQ ID NO: 49 - DNA, 28 bp, Artificial Sequence
  human metapneumovirus reverse primer
cctatytcwgcagcatatttgtaatcag

SEQ ID NO: 50 - DNA, 28 bp, Artificial Sequence
  human metapneumovirus probe
caachgcagtracaccytcatcattrca

SEQ ID NO: 51 - DNA, 27 bp, Artificial Sequence
  respiratory syncytial virus forward primer
cttagcaaagtcaagttraatgataca

SEQ ID NO: 52 - DNA, 23 bp, Artificial Sequence
  respiratory syncytial virus reverse primer
tgcacatcataattrggagtgtc

SEQ ID NO: 53 - DNA, 23 bp, Artificial Sequence
  respiratory syncytial virus probe
acyatycaacgkagyacaggaga

SEQ ID NO: 54 - DNA, 23 bp, Artificial Sequence
  Parainfluenza virus type I forward primer
ggaggagcaattatacctggtca

SEQ ID NO: 55 - DNA, 20 bp, ARtificial Sequence
  parainfluenza virus type I reverse primer
tgtatccartgagtgggcta

SEQ ID NO: 56 - DNA, 27 bp, Artificial Sequence
  parainfluenza virus type I probe
attaggcccgagtgtracrgatgatgc

SEQ ID NO: 57 - DNA, 21 bp, Artificial Sequence
  parainfluenza virus type 2 forward primer
tatgcyatggtgggagacatt

SEQ ID NO: 58 - DNA, 21 bp, Artificial Sequence
  parainfluenza viue type 2 reverse primer
gccatcttgttccaagtccat

SEQ ID NO: 59 - DNA, 29 bp, Artificial Sequence
  parainfluenza type 2 probe
cctcccattccgctgtgttcaatrtactt

SEQ ID NO: 60 - DNA, 23 bp, Artificial Sequence
  parainfluenza type 3 forward primer
agctatyactagyatctcagggt

SEQ ID NO: 61 - DNA, 20 bp, Artificial Sequence
  parainfluenza type 3 reverse primer
cccaatctgatccactgtgt

SEQ ID NO: 62 - DNA, 26 bp, Artificial Sequence
  parainfluenza type 3 probe
tcagacaagatggaacagtgcaggca